(19) United States
(12) Patent Application Publication
(10) Pub. No.: US 2007/0105810 A1
(43) Pub. Date: May 10, 2007

(54) THERAPEUTICS

(76) Inventors: Barry Victor Lloyd Potter, Bath (GB); James Dowden, Nottingham (GB); Antony Galione, Oxford (GB); Andreas H. Guse, Hamburg (DE); Alexander Flugel, Martinsried (DE)

Correspondence Address: FROMMER LAWRENCE & HAUG, 745 FIFTH AVENUE- 10TH FL., NEW YORK, NY, 10151, US

(21) Appl. No.: 11/422,010

(22) Filed: Jun. 2, 2006

Related U.S. Application Data
(63) Continuation-in-part of application No. PCT/GB04/05109, filed on Dec. 6, 2004.
(60) Provisional application No. 60/691,028, filed on Jun. 15, 2005.

(30) Foreign Application Priority Data
Dec. 5, 2003 (GB) 0328314.0

(51) Int. Cl. A61K 31/70; A01N 43/04

(52) U.S. Cl. 514/45

(57) ABSTRACT

The present invention relates to the use of a compound of formula (1): 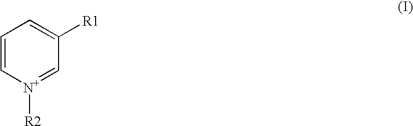 wherein: R1 comprises a carbonyl group and R2 is a hydrocarbyl group; optionally wherein said ring is further substituted; or a pharmaceutically acceptable salt thereof; in the manufacture of a medicament for use in one or more of: modulating the release of intracellular calcium from a store controlled by nicotinic acid adenine dinucleotide phosphate; modulating calcium spikes in mammalian cells; treating diseases in one or more of brain, heart, pancreatic cells (e.g. pancreatic acinar and pancreatic beta cells), immune cells, T-cells, haemopoietic cells including phagocytes; treating diseases in one or more of brain, heart, pancreatic cells (e.g. pancreatic acinar and pancreatic beta cells), immune cells, T-cells, haemopoietic cells including phagocytes by modulating the release of intracellular calcium from a store controlled by nicotinic acid adenine dinucleotide phosphate; treating diseases in one or more of brain, heart, and T-cells by modulating calcium spikes in mammalian cells.

Reference is made to PCT application PCT/GB2004/005109 having a filing date of 6 Dec. 2004 and a priority date of 5 Dec. 2003 from GB patent application GB0328314.0—wherein said PCT designated all contracting states including the USA. PCT/GB2004/005109 and GB0328314.0 are incorporated by reference herein in their entirety.

Reference is also made to U.S. patent application U.S. Ser. No. 60/691,028 having a filing date of 15 Jun. 2005 and is incorporated by reference herein in its entirety.

Reference is also made to U.S. patent application U.S. Ser. No. 60/______ (to be assigned, provisional application filed May 15, 2006 designated by attorney docket number 674177-2001.2) having a filing date of 15 May 2006 and is incorporated by reference herein in its entirety.

It is noted that in this disclosure, terms such as “comprises”, “comprised”, “comprising”, “contains”, “containing” and the like can have the meaning attributed to them in U.S. patent law; e.g., they can mean “includes”, “included”, “including” and the like. Terms such as “consisting essentially of” and “consists essentially of” have the meaning attributed to them in U.S. patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. patent law; namely, that these terms are closed ended.

### FIELD OF THE INVENTION

The present invention relates to therapeutics.

In particular, but not exclusively, the present invention relates to therapeutics—such as compounds and compositions—for modulating the release of intracellular calcium from a store controlled by nicotinic acid adenine dinucleotide phosphate; modulating calcium spikes in mammalian cells; treating diseases in one or more of brain, heart, pancreatic cells (e.g. pancreatic acinar and pancreatic beta cells), immune cells, T-cells, haemopoietic cells including phagocytes; treating diseases in one or more of brain, heart, pancreatic cells (e.g. pancreatic acinar and pancreatic beta cells), immune cells, T-cells, haemopoietic cells including phagocytes by modulating the release of intracellular calcium from a store controlled by nicotinic acid adenine dinucleotide phosphate; and/or treating diseases in one or more of brain, heart, pancreatic cells (e.g. pancreatic acinar and pancreatic beta cells), immune cells, T-cells, haemopoietic cells including phagocytes by modulating calcium spikes in mammalian cells.

### BACKGROUND TO THE INVENTION

Calcium plays a pivotal role within the cell from initial fertilization of an egg, through every day functions, to cell death. Exactly how calcium exerts such remarkable specificity is the subject of intense study.

Inositol 1,4,5-trisphosphate (IP₃) and cADP-ribose (cADPr) are well-accepted as mediators of Ca²⁺ release from intracellular stores. However, the role of a third putative Ca²⁺-mobilizing molecule, nicotinic acid-adenine dinucleotide phosphate (NAADP), has until recently been more controversial.

Of all the Ca²⁺ mobilizing molecules considered as intracellular messengers, NAADP is the most potent. Typically, nanomolar concentrations in the cytoplasm lead to the release of Ca²⁺ from intracellular stores. By way of example, evidence in the sea urchin egg has been obtained to show that the major NAADP-sensitive Ca²⁺-pool is non-endoplasmic reticular in nature.

NAADP was first discovered as a contaminant in NADP that has Ca²⁺-releasing activity on sea urchin egg microsomes. Synthesis of NAADP involves a modification of the enzymatic reaction, resulting in the exchange of nicotinamide for nicotinic acid on the unphosphorylated ribose ring of NADP. The latter mechanism is favoured at low pHs such as those pertaining in an endosomal compartments where CD38 may reside during recycling from the cell surface. Replacement of an uncharged amide in NADP for a negatively charge carboxyl function in NAADP confers on the latter a potent (nanomolar affinity) capacity to mobilize Ca²⁺ from responsive stores.

NAADP mobilizes intracellular Ca²⁺ stores in several cell types. For example, NAADP mobilizes Ca²⁺ in a number of systems from higher organisms, including mammalian T-lymphocytes, pancreatic, kidney and heart cells. The pancreatic acinar cells were the first reported, where NAADP was found to be responsible for initiating the cholecystokinin-induced Ca²⁺ signals. Likewise, in pancreatic β-cells, NAADP-sensitive Ca²⁺ stores also play important roles in mediating Ca²⁺ signalling activated by insulin and glucose.

It is also known that NAADP+ specifically and dose-dependently stimulates Ca²⁺ signalling in human T cells (WO 02/11736).

Evidence suggests that NAADP activates intracellular Ca²⁺ channels distinct from those that are sensitive to inositol trisphosphate and ryanodine/cyclic ADP-ribose. Recent studies in intact cells have demonstrated functional coupling between Ca²⁺ release pathways mediated by NAADP, inositol trisphosphate and cyclic ADP-ribose. Thus, NAADP is an important determinant in shaping cytosolic Ca²⁺ signals.

A specific binding site for NAADP has been identified in sea urchin egg preparations (J. Biol. Chem (1996) 271 8531-8516; Biochem J. (2000) 352, 725-729) and evidence that this binding site is related to NAADP induced Ca²⁺ mobilization has also been obtained.

The role of NAADP receptors in the co-ordination of calcium signalling has been reviewed in Trends Biochem Sci. (2001) 26(8):482-9 and Cell Calcium (2002) 32(5-6):343-54.

Despite the importance of NAADP as a Ca⁺-mobilizing molecule, there are at present no small molecules that selectively affect the NAADP binding site. Consequently there are no examples of any compounds that exploit such activity as therapeutic agents.

### SUMMARY OF THE INVENTION

The present invention is based in part upon the development of chemical entities that modulate the release of intracellular calcium from a specific store controlled by NAADP. Typically, these molecules are small molecules with a RMM of <500 and are cell permeable. Advantageously, these and related compounds may find application as novel therapeutic agents and as probes for biological assays.

These molecules will help to solve the technical problems associated with further characterisation of this biological pathway by providing readily available small chemical tools. To date, no cell permeable molecules have been available to scientists working in the field and this has hampered biological studies.

Aspects and embodiments of the present invention are presented in the accompanying claims and in the following description and discussion. These aspects are presented under separate section headings. However, it is to be understood that the teachings under each section heading are not necessarily limited to that particular section heading.

### ASPECTS OF THE INVENTION

Aspects of the present invention include:

The use of a compound of formula (I): 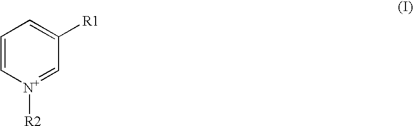
wherein:
R1 comprises a carbonyl group
R2 is a hydrocarbyl group;
   optionally wherein said ring is further substituted;
   or a pharmaceutically acceptable salt thereof;
   in the manufacture of a medicament for use in one or more of:
modulating the release of intracellular calcium from a store controlled by nicotinic acid adenine dinucleotide phosphate
modulating calcium spikes in mammalian cells
treating diseases in one or more of brain, heart, pancreatic cells (e.g. pancreatic acinar and pancreatic beta cells), immune cells, T-cells, haemopoietic cells including phagocytes
treating diseases in one or more of brain, heart, pancreatic cells (e.g. pancreatic acinar and pancreatic beta cells), immune cells, T-cells, haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), immune cells, T-cells, haemopoietic cells including phagocytes by modulating calcium spikes in mammalian cells.

Examples of optional ring substituents (sometimes referred to as R3) include one or more H, a substituted or unsubstituted aryl (e.g. C1-15 aryl), C1-20 alkyl, X (e.g. F, Cl, Br, I), OY, NYₙ (n=1, 2, or 3), SY, COY, wherein Y is H, substituted or unsubstituted aryl (e.g. C1-15 aryl), C1-20 alkyl. Thus, in the formulae presented in the claims, R3 represents one or more substituents, the or each substituent being independently selected from H, substituted or unsubstituted aryl, C1-20 alkyl, F, Cl, Br, I, OY, NYₙ (n=1, 2, or 3), SY, COY, CONY_{z} (z=2), C(O)OY, wherein the or each Y is independently selected from H, a substituted or unsubstituted aryl, and a C1-20 alkyl group. R3 may be selected from one or more H, C1-15 aryl, C1-20 alkyl, X (e.g. F, Cl, Br, I), OY, NYₙ (n=1, 2, or 3), SY, COY, wherein Y is H, C1-15 aryl, C1-20 alkyl.

The optional ring substituents, R3, may comprise a substituted or unsubstituted aryl group. A substituted aryl group comprises one or more independently selected substituents on an aryl ring system. In particular, the substituted aryl group may comprise one or more hydroxy; alkyl, especially lower (C₁-C₆) alkyl (e.g. methyl, ethyl, n-propyl, isopropyl, n-butyl, sec-butyl, tert-butyl, n-pentyl and other pentyl isomers, and n-hexyl and other hexyl isomers); alkoxy, especially lower (C₁-C₆) alkoxy (e.g. methoxy, ethoxy, propoxy etc.); alkinyl (e.g. ethinyl); or halogen (e.g. fluoro, chloro, bromo, iodo) substituents. Preferably the aryl ring system is a C3-C15 aryl ring system. Preferably the aryl ring system is a C6-C15 aryl ring system.

A pharmaceutical composition comprising a compound as defined herein or a pharmaceutically acceptable salt thereof admixed with a pharmaceutically acceptable carrier, diluent or excipient.

A compound of formula (I) or a pharmaceutically acceptable salt thereof as defined herein for use in medicine.

A compound of formula (I) or a pharmaceutically acceptable salt thereof.

A medicament comprising a compound as defined herein.

An assay method for identifying an agent that modulates intracellular calcium release comprising the steps of:
(a) providing an agent;
(b) providing an NAADP receptor;
(c) contacting said agent with an NAADP receptor; and
(d) measuring the level of intracellular calcium release;
   wherein a difference between (i) the level of intracellular calcium release in the presence of the agent; and (ii) the level of intracellular calcium release in the absence of the agent is indicative of an agent that modulates intracellular calcium release and may be useful in one or more of:
modulating the release of intracellular calcium from a store controlled by nicotinic acid adenine dinucleotide phosphate;
   modulating calcium spikes in mammalian cells;
treating diseases in one or more of brain, heart, pancreatic cells (e.g. pancreatic acinar and pancreatic beta cells), immune cells, T-cells, haemopoietic cells including phagocytes;
treating diseases in one or more of brain, heart, pancreatic cells (e.g. pancreatic acinar and pancreatic beta cells), immune cells, T-cells, haemopoietic cells including phagocytes by modulating the release of intracellular calcium from a store controlled by nicotinic acid adenine dinucleotide phosphate; and
treating diseases in one or more of brain, heart, pancreatic cells (e.g. pancreatic acinar and pancreatic beta cells), immune cells, T-cells, haemopoietic cells including phagocytes by modulating calcium spikes in mammalian cells.

An assay method comprising the steps of:
(a) performing the assay method as defined herein;
(b) identifying one or more agents capable of modulating intracellular calcium release; and
(c) preparing a quantity of those one or more identified agents.

A method comprising the steps of:
(a) performing the assay method as defined herein;
(b) identifying one or more agents capable of modulating intracellular calcium release; and
(c) preparing a pharmaceutical composition comprising those one or more identified agents.

An agent identifiable, preferably, identified by the assay method as defined herein.

A pharmaceutical composition prepared by the method as defined herein.

A method of treating and/or preventing a disease in a human or animal patient in need of same which method comprises administering to the patient an effective amount of a compound as defined herein, a composition as defined herein, or a medicament as defined herein.

A process of preparing a pharmaceutical composition, said process comprising admixing one or more of the compounds as defined herein with a pharmaceutically acceptable diluent, excipient or carrier.

A pharmaceutical pack comprising one or more compartments, wherein at least one compartment comprises one or more of the compounds as defined herein, a composition as defined herein, or a medicament as defined herein.

A container comprising a compound as defined herein, a composition as defined herein, or a medicament as defined herein, wherein said container is labelled for use in one or more of:
modulating the release of intracellular calcium from a store controlled by nicotinic acid adenine dinucleotide phosphate
modulating calcium spikes in mammalian cells
treating diseases in one or more of brain, heart, pancreatic cells (e.g. pancreatic acinar and pancreatic beta cells), immune cells, T-cells, haemopoietic cells including phagocytes
treating diseases in one or more of brain, heart, pancreatic cells (e.g. pancreatic acinar and pancreatic beta cells), immune cells, T-cells, haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), immune cells, T-cells, haemopoietic cells including phagocytes by modulating calcium spikes in mammalian cells.

The use of a compound of formula (I) in the manufacture of a medicament for use in one or more of:
modulating the release of intracellular calcium from a store controlled by nicotinic acid adenine dinucleotide phosphate
modulating calcium spikes in mammalian cells
treating diseases in one or more of brain, heart, pancreatic cells (e.g. pancreatic acinar and pancreatic beta cells), immune cells, T-cells, haemopoietic cells including phagocytes
treating diseases in one or more of brain, heart, pancreatic cells (e.g. pancreatic acinar and pancreatic beta cells), immune cells, T-cells, haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar cells) and T-cells by modulating calcium spikes in mammalian cells.
   wherein said compound is cell permeable;
   wherein said compound has a relative molecular mass of less than about 500;
   wherein said compound is a mimetic of the nicotinic group of NAADP, wherein said NAADP has the formula (II):

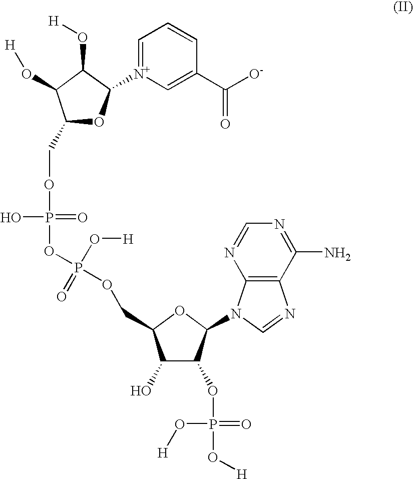

In a further aspect, said compound, that is a mimetic of the nicotinic group of NAADP, has the formula (A): 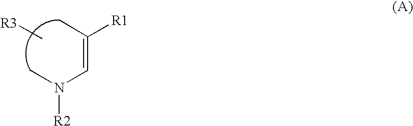
wherein R1 and R2 are ring substituents;
wherein R1 and R2 are as defined for compound of formula (I); and
wherein R3 is as defined herein.

In addition to compounds of formula (A), the present invention also relates to pharmaceutically acceptable salts thereof.

### SOME PREFERRED ASPECTS OF THE INVENTION

Preferably, said compound is cell permeable.

Preferably, said compound has a relative molecular mass (RMM) of less than about 500.

Preferably, R1 is —C(O)R4 wherein R4 is —OH, —O-hydrocarbyl, or —O—N(R5)(R6); wherein each of R5 and R6 is independently selected from H or a hydrocarbyl.

Preferably, R1 is a hydrocarbyl group comprising a carbonyl group, or COOH.

Preferably, R2 is hydrocarbyl, hydrocarbyl-N, hydrocarbyl-N(R7)(R8), or hydrocarbyl-C(O)—N(R9)(R10); wherein each of R7, R8, R9 and R10 is independently selected from H or a hydrocarbyl.

Preferably, R2 is a hydrocarbyl group comprising a carbonyl group.

Preferably, R2 is a hydrocarbyl group comprising an amide group.

Preferably, R2 is a group comprising—hydrocarbyl-C(O)N(H or hydrocarbyl)(H or hydrocarbyl).

Preferably, R2 is a group comprising —CH₂C(O)O)N(H or hydrocarbyl)(H or hydrocarbyl).

Preferably, R2 is a group comprising —CH₂C(O)NH₂.

Preferably, said compound is a mimetic of nicotinic group of NAADP, wherein said NAADP has the formula (II):

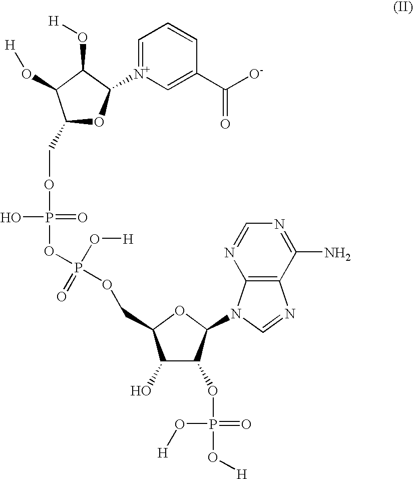

Preferably, the present invention relates to the use of said compounds in the manufacture of a medicament for use in one or more of treating an autoimmune disease (such as thyroiditis, insulitis, multiple sclerosis, invectitis, orchitis, myasthenia gravis, rhematoid arthritis or lupus erythematosis) or graft rejection, or Type II diabetes, or cardiac arrhythmia, or treating or preventing an immune disorder in a human or animal.

Preferably, the present invention relates to the use of said compounds in the manufacture of a medicament for use in one or more of treating thyroiditis, insulitis, multiple sclerosis, invectitis, orchitis, myasthenia gravis, rhematoid arthritis, lupus erythematosis, graft rejection, Type II diabetes or cardiac arrhythmia.

### SOME ADDITIONAL ASPECTS OF THE COMPOUNDS OF THE INVENTION

The ring structure of the compound of formula (1) may be further substituted—such as with another hydrocarbyl group. By way of example, such substituents may be a halo group and/or a hydrocarbyl group.

### SOME EXEMPLARY COMPOUNDS OF THE INVENTION

Exemplary compounds of the present invention include the following (which are shown as salt forms—however the non-salt forms are also covered as well as other salt forms):

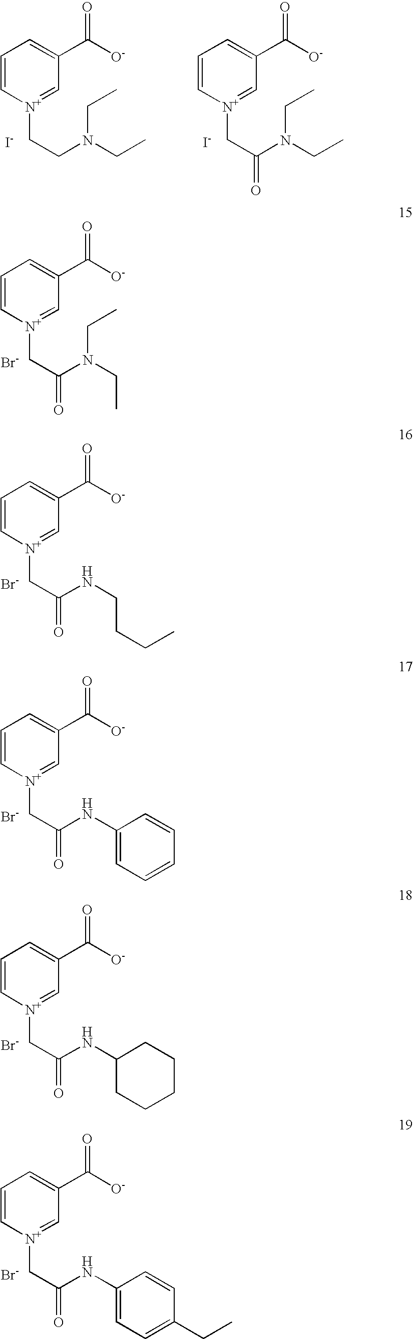

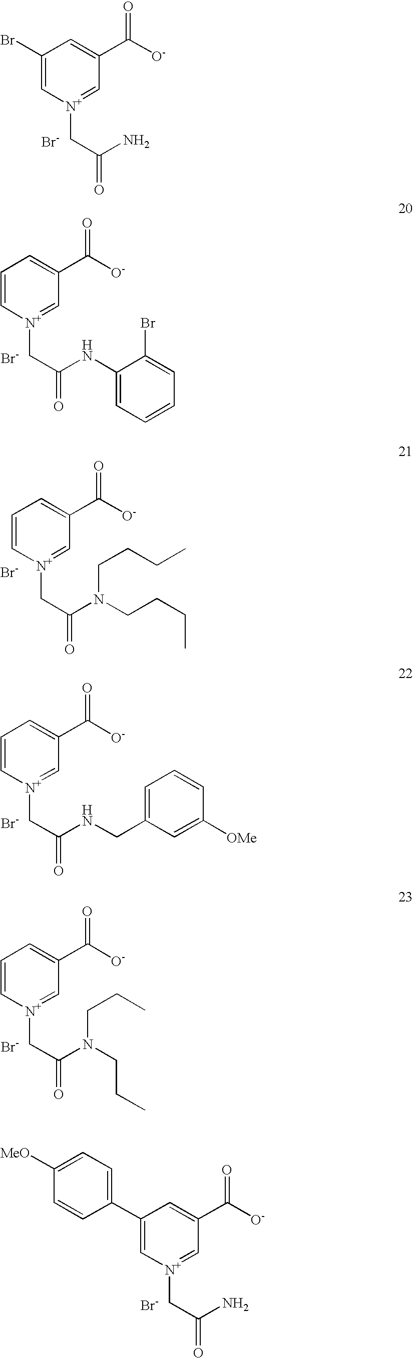

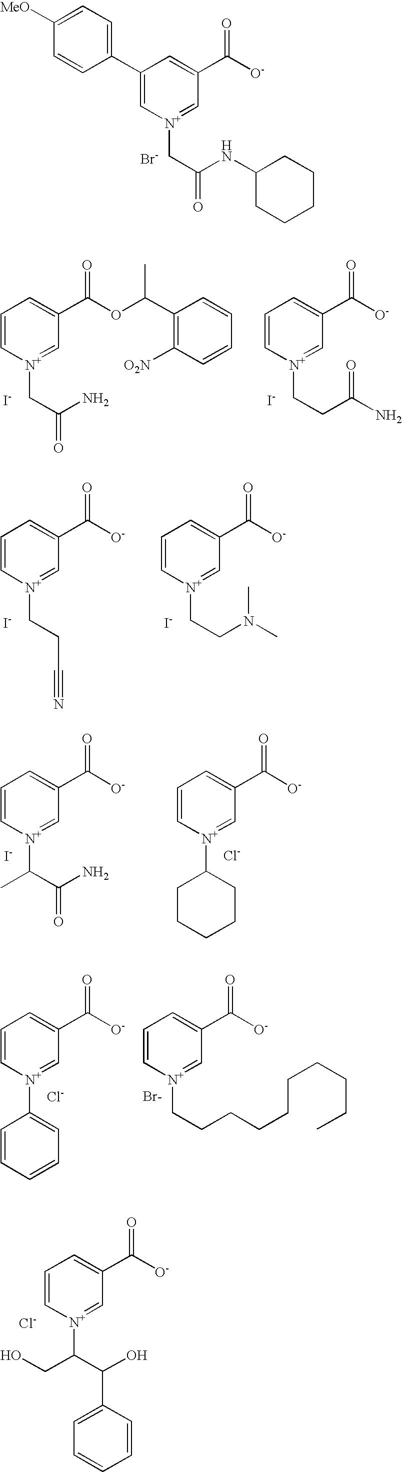

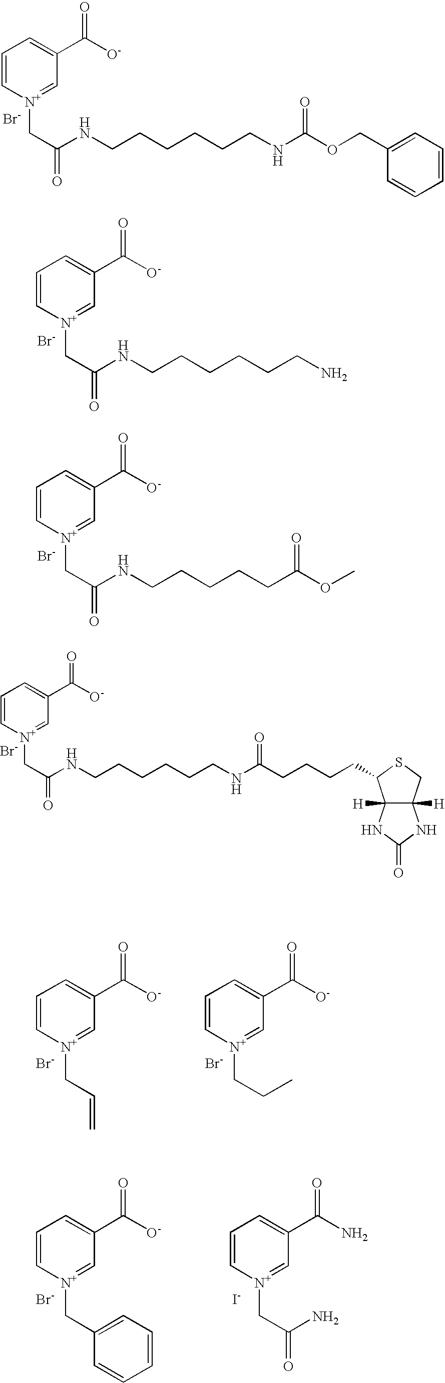

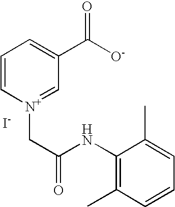

### ADVANTAGES

The present invention has a number of advantages. These advantages will be apparent in the following description.

By way of example, the present invention is advantageous since it provides commercially useful compounds, compositions and methods.

By way of example, the present invention is advantageous since it provides commercially useful compounds, compositions and methods that selectively affect the NAADP binding site.

By way of example, the present invention is advantageous since it provides commercially useful compounds, compositions that may be used as therapeutic agents.

### DESCRIPTION OF THE FIGURES

FIG. 1

[³²P]NAADP binding to 0.5% sea urchin egg homogenate.

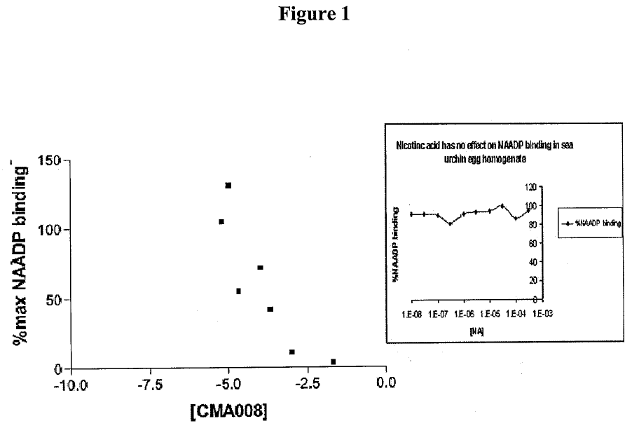

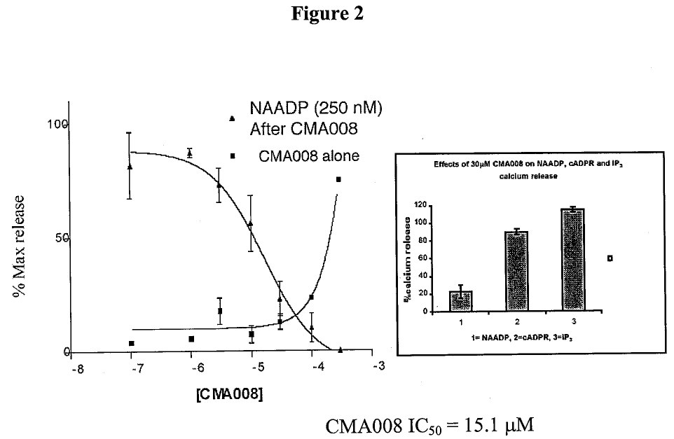

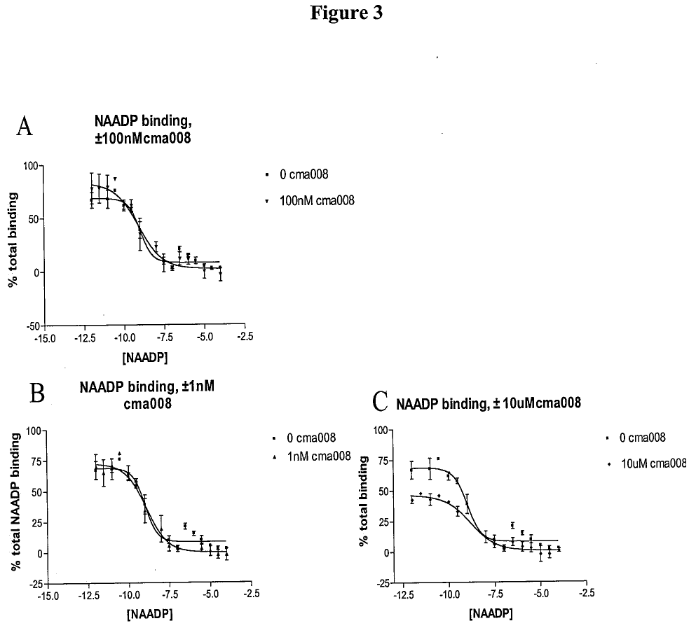

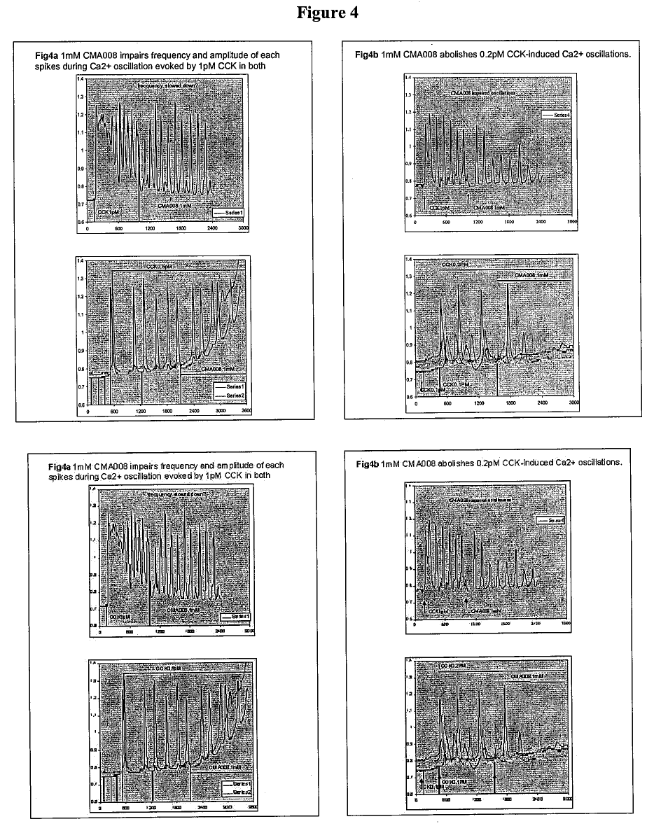

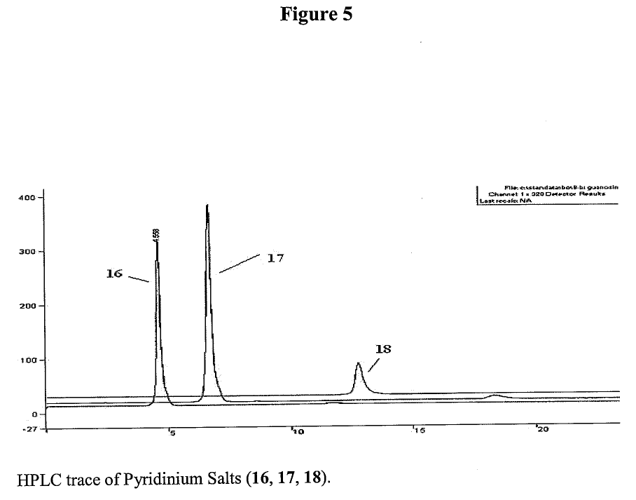

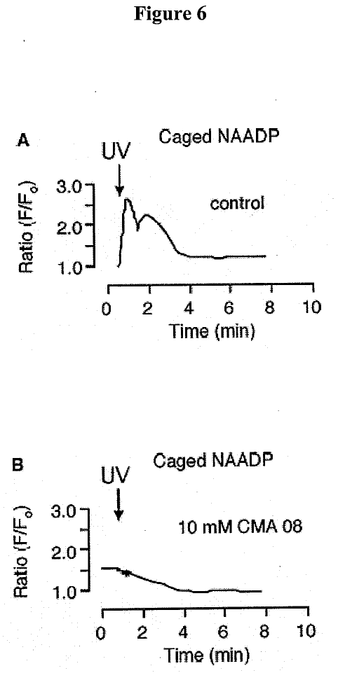

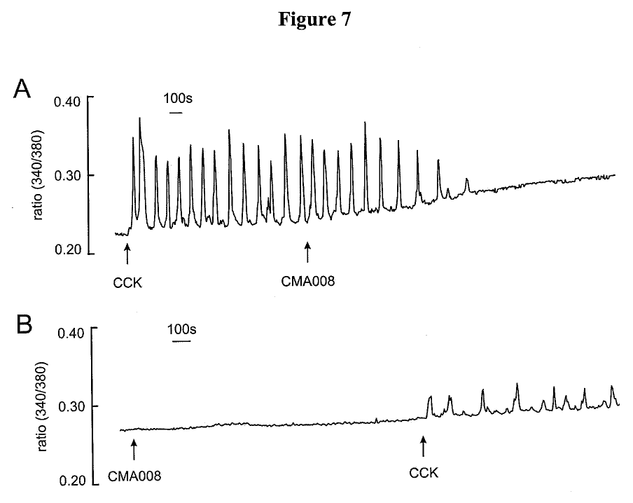

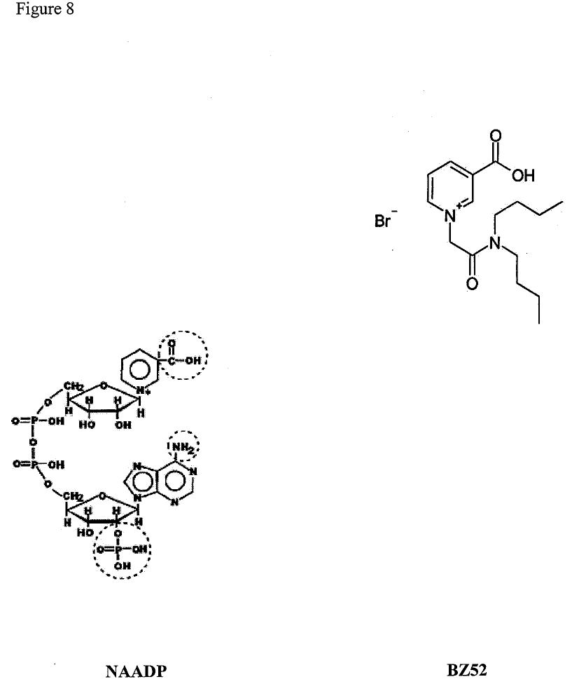

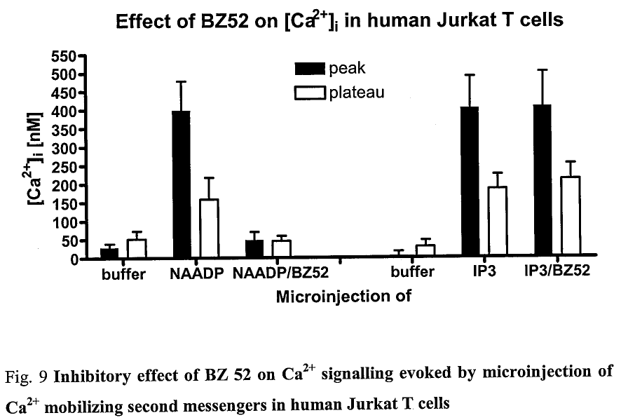

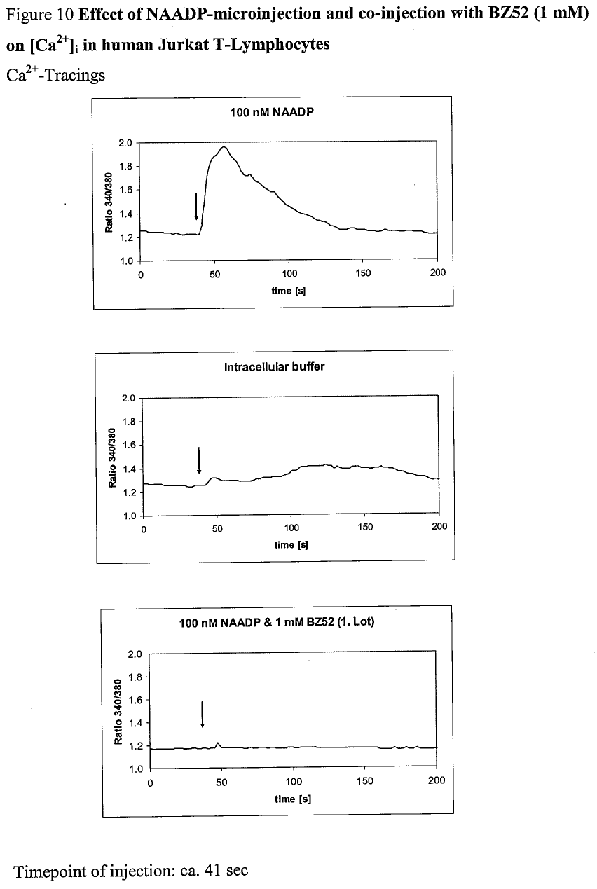

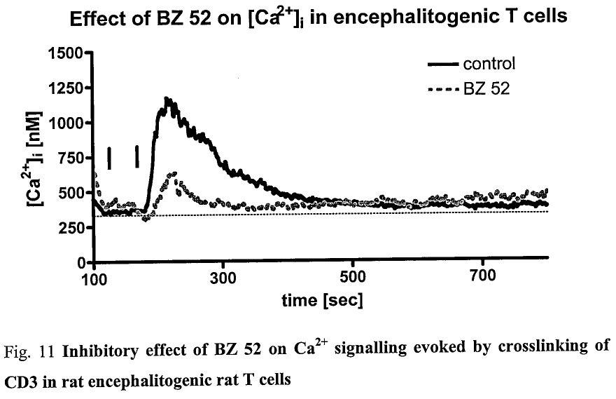

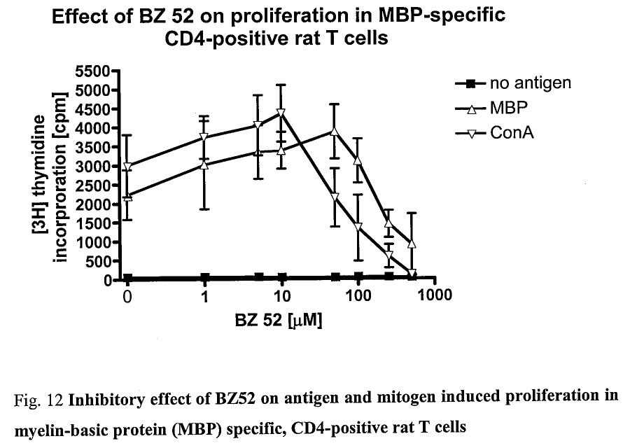

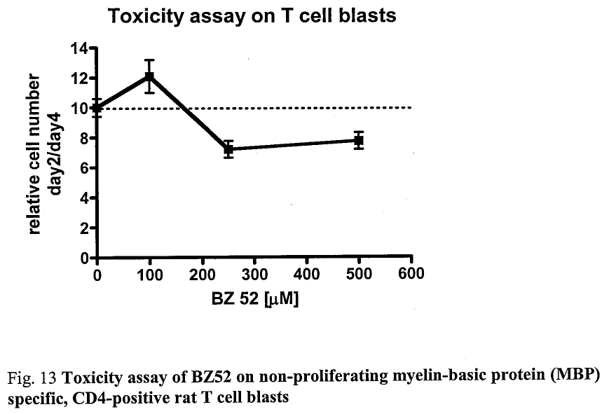

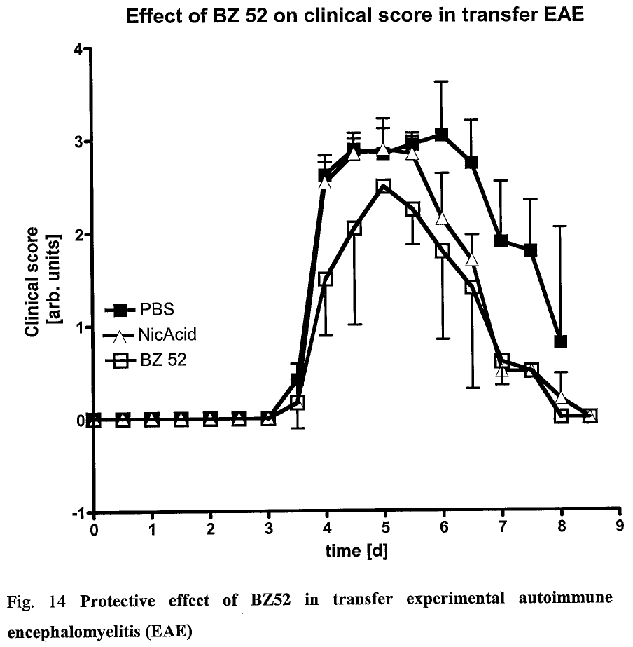

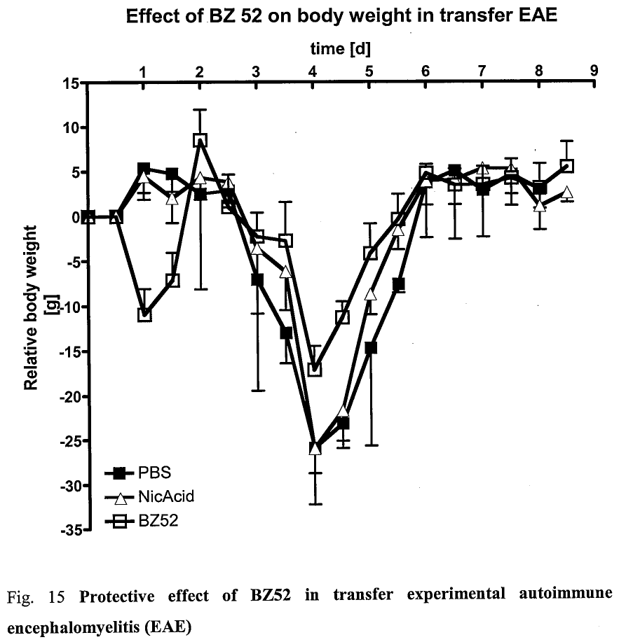

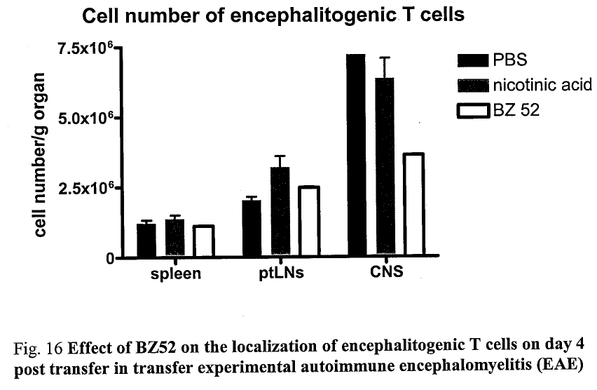

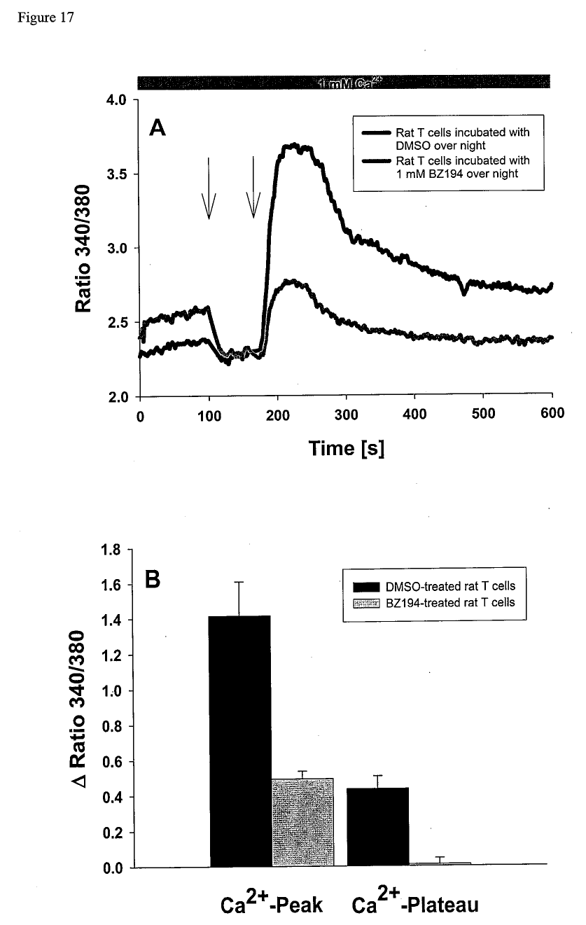

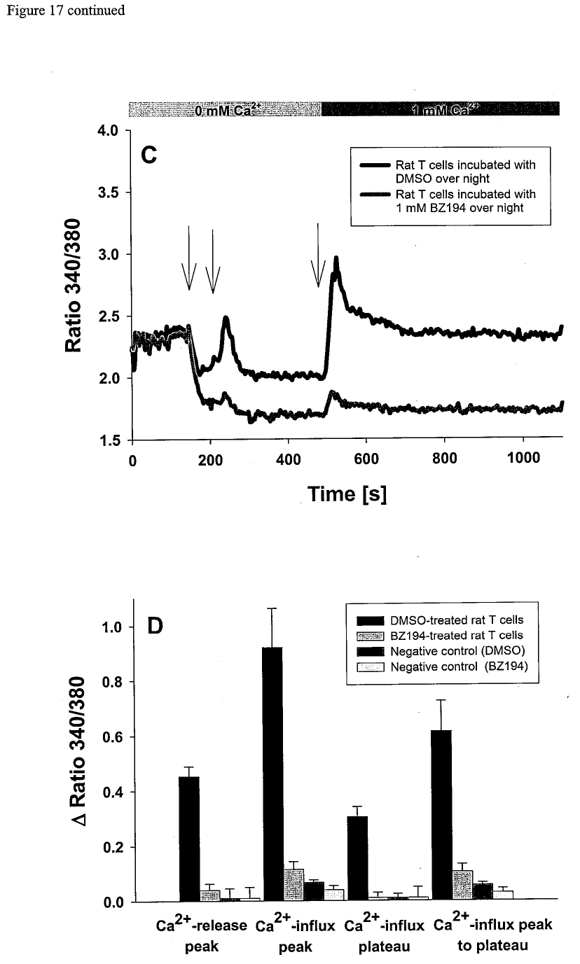

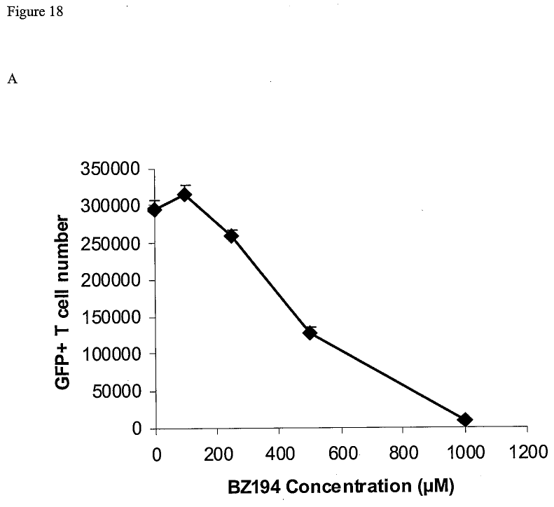

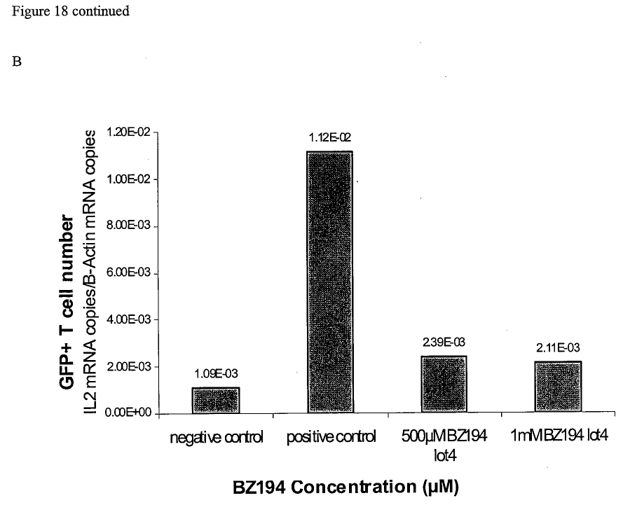

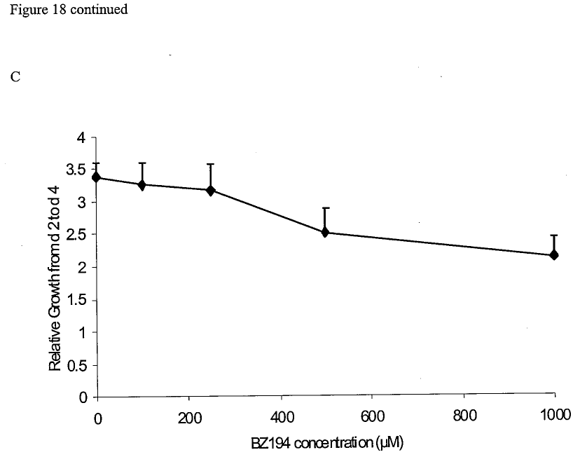

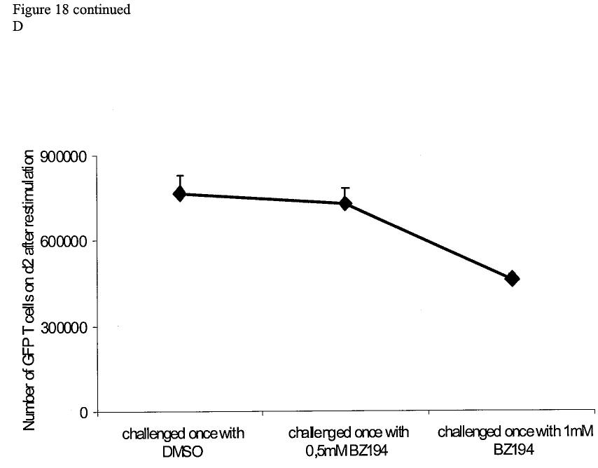

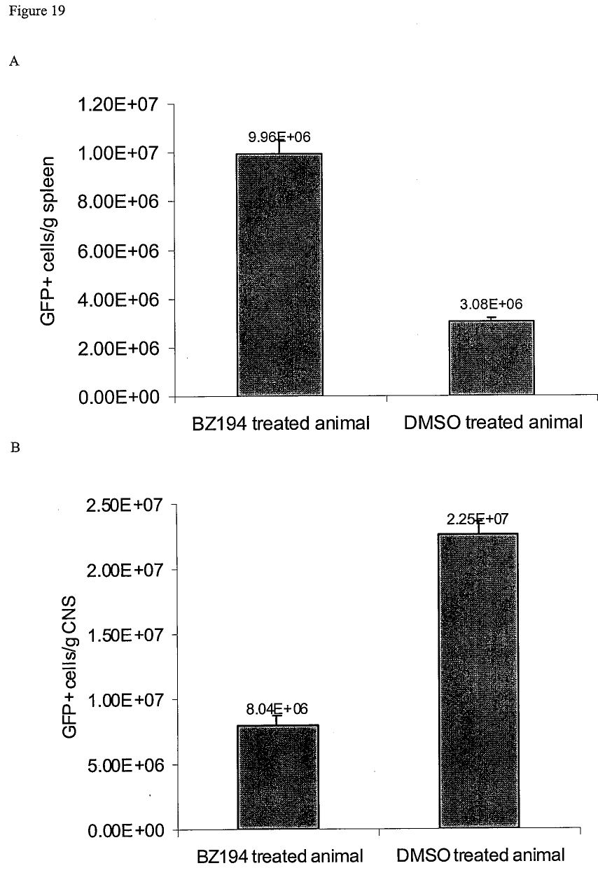

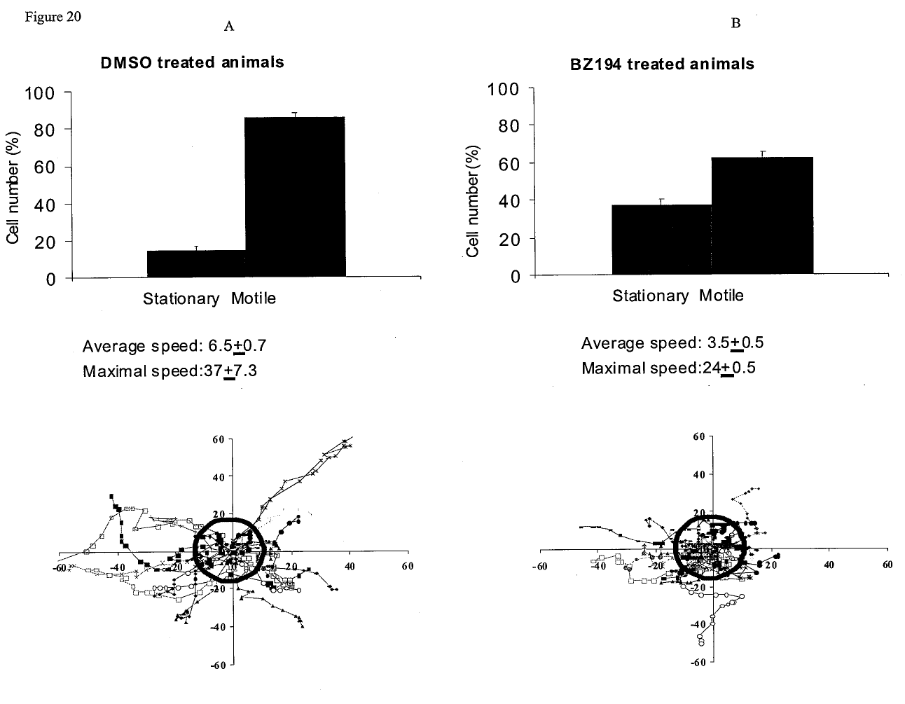

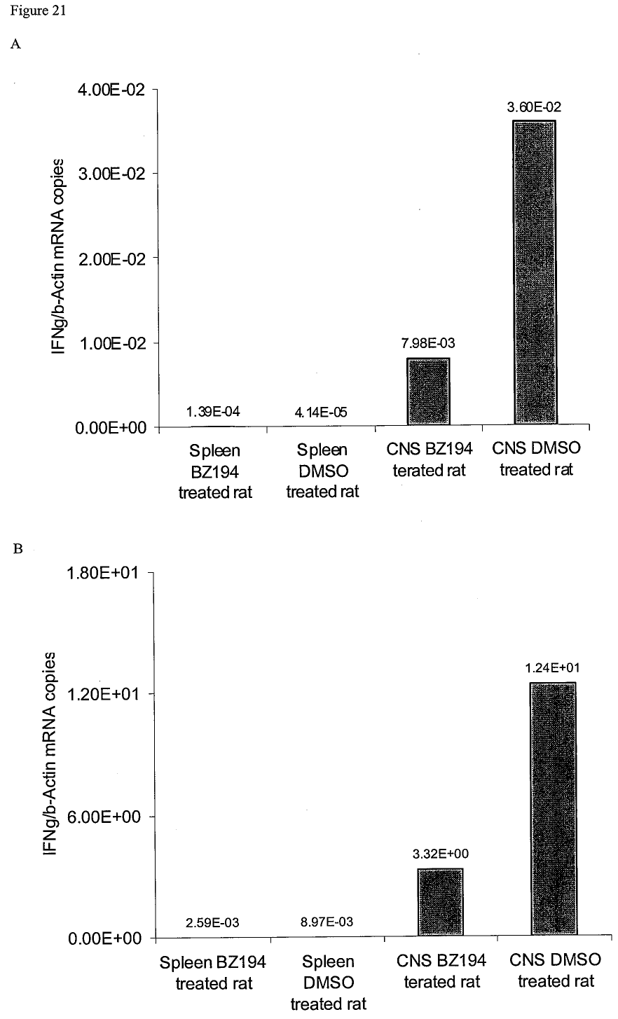

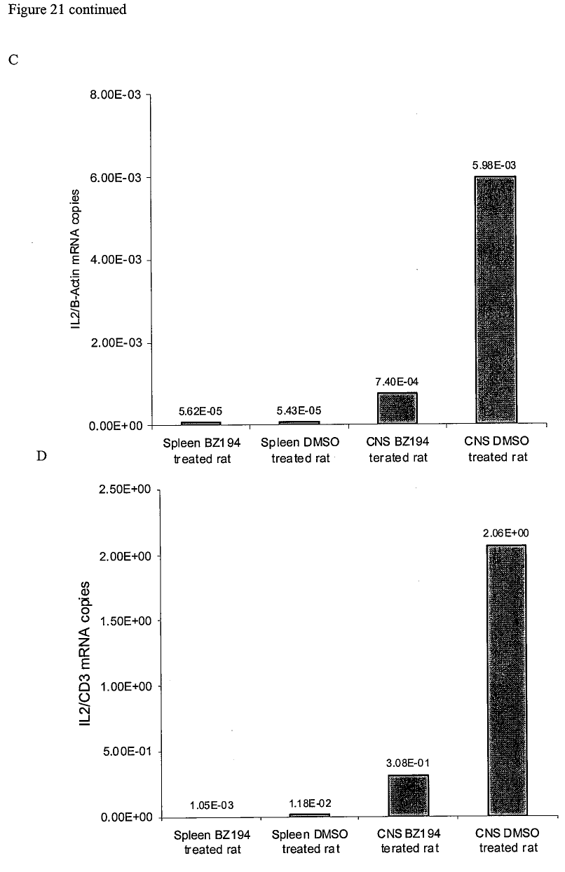

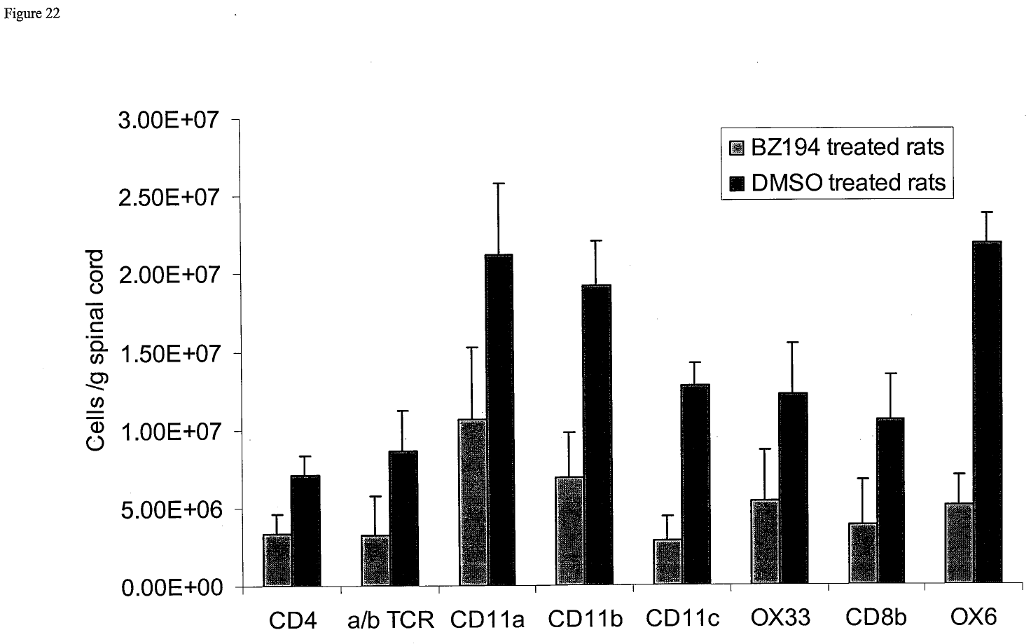

Competitive CMA008 (1-Carbamoylmethyl-3-carboxy-pyridinium iodide) displacement of [³²P]NAADP (0.2 nM). Samples diluted in GluIM (intracellular medium), pre-incubated with indicated concentration of CMA008 for 10 minutes, then 0.2 nM [³²P]NAADP added and incubated at room temperature for a further 15 mins. Samples filtered through Whatman GF/B filters to separate bound and free [³²P]NAADP ligand. Non specific binding is defined by incubation of the homogenate in the presence of 10 μM NAADP. n=2. Data expressed as a fraction of total binding. The inset in FIG. 1 shows that adding nicotinic acid (up to 1 mM) was without effect on [³²P]NAADP binding to egg membranes.

FIG. 2

CMA008 (1-Carbamoylmethyl-3-carboxy-pyridinium iodide) inhibits NAADP-mediated calcium release in sea urchin egg homogenate.

Samples diluted to 2.5% in GluIM in the presence of regenerating system and kept at 17° C. with agitation for 3 hours to facilitate calcium uptake into stores. Calcium release was determined by measuring increase in Fluo-3 fluorescence at 526 nm. Data expressed as % of 250 nM NAADP calcium release in the absence of CMA008 (1-Carbamoylmethyl-3-carboxy-pyridinium iodide). CMA008 inhibits NAADP calcium mobilization with an IC₅₀=15.13 μM. CMA008 has no effect on the total calcium mobilization by IP3 or cADPR (insert) indicating the selectivity of this compound on the NAADP-sensitive release mechanism.

FIG. 3

Effects of CMA008 (1-Carbamoylmethyl-3-carboxy-pyridinium iodide) on the competition of [³²P]NAADP in the presence of increasing concentrations of unlabelled NAADP.

2.5% sea urchin egg homogenate was diluted into GluIM pre-incubated with indicated concentration of CMA008 for 10 minutes at room temperature (A=100 nM, B=1 nM, C=10 μM). Subsequently, [³²P]NAADP was added and samples incubated for a further 15 minutes at room temperature. Samples were filtered through Whatman GF/B filters to separate bound and free [³²P]NAADP ligand. CMA008 reduces the Bₘₐₓ from 68.65%±2.7 total specific binding in the absence of CMA008 to 46.28%±2.61 in the presence of 10 μM CMA008. 1 nM and 100 nM CMA008 had no effect on the Bₘₐₓ. There is no effect on the IC₅₀ for NAADP at any of the tested concentrations. n is between 2 and 6 for each point.

FIG. 4

Effect of CMA008 (1-Carbamoylmethyl-3-carboxy-pyridinium iodide) on CCK-induced oscillations of Ca²⁺ in pancreatic acinar cells

Pancreatic acinar cells were seeded onto poly-lysine-coated number 1 glass coverslips and loaded by incubating cells with 1-5 μM fura-2 acetoxymethylester (Molecular Probes; Leiden, Holland) for 60 min at room temperature. After the loading period, cells were subsequently washed and maintained in buffer at room temperature and used immediately. Cells were excited alternately with 340 and 380 nm light (emission 510 nm), and ratio image of clusters were recorded every 4-5 s, using a 12-bit CCD camera (MicroMax; Princeton Instruments, NJ). All experiment were conducted at room temperature. CMA008 (1-Carbamoylmethyl-3-carboxy-pyridinium iodide) is dissolved in 50% DMSO. Final concentration of DMSO is 0.5% in the solution (composition in mM: 140 NaCl, 4.7 KCl, 1 CaCl2, 1.13 MgCl2, 10 HEPES, 10 Glucose, pH adjusted to 7.2)

FIG. 5

HPLC of the pyridinium salts.

FIG. 6

Abolition of calcium release in response to uncaging of caged NAADP in intact sea urchin eggs (loaded with the calcium reporter dye calcium green dextran) with external application of CMA008.

Sea urchin eggs were microinjected with a solution containing the calcium reporter dye, calcium green dextran, together with caged NAADP. Fluorescence was then imaged on a Leica confocal microscope using an excitation wavelength of 488 nm. In the control egg [see FIG. 6 A] a brief UV flash photolysed a proportion of the caged NAADP and evoked a large calcium transient. However, when eggs were incubated with sea water containing CMA008 (10 mM) [see FIG. 6 B], liberation of NAADP by photolysis failed to induce and calcium release. This indicates that CMA008 permeates the cell membrane and inhibits NAADP-induced calcium release.

FIG. 7

Cholecystokinin (CCK)-evoked calcium oscillations (dependent on NAADP signalling) in isolated mouse pancreatic cells are inhibited or reduced by external application of CM008.

Traces show the Ca²⁺ dye fluorescence.

Pancreatic acinar cells were isolated from mice and dispersed by collagenase treatment. Cells were incubated with fura-2 AM for 30 min and washed before being imaged on a Metafluor system. The cells were alternately excited at 340/380 nm and emitted light collected at around 510 nm. The ratio of the intensities of emitted light at the two excitation wavelengths were calculated, converted to free calcium concentrations, and plotted again time.

In FIG. 7 A, the peptide cholecystokinin (CCK; 5 pM) was added to the cells and resulted in a robust series of calcium spikes in the continued presence of the agonist. However, when CMA008 (1 mM) was added to the bathing solution (i.e. post-CCK addition), it resulted in the inhibition of CCK-evoked calcium spiking. In FIG. 7 B, the CMA008 (1 mM) was added first to the medium (i.e. pre-CCK addition) with no apparent effect on resting calcium levels. However, now on application of CCK, a much smaller response to CCK was seen, with a greatly reduced frequency. Since it has been shown that NAADP is a critical messenger for CCK-mediated calcium signalling, it appears that CMA008 is able to enter pancreatic acinar cells where it may block NAADP-evoked calcium release.

### DETAILED DESCRIPTION OF THE INVENTION

Substituents

The compounds of the present invention may have substituents other than those of the ring systems shown herein. Furthermore the ring systems herein are given as general formulae and should be interpreted as such. The absence of any specifically shown substituents on a given ring member indicates that the ring member may be substituted with any moiety of which H is only one example. The ring system may contain one or more degrees of unsaturation, for example in some aspects one or more rings of the ring system is aromatic. The ring system may be carbocyclic or may contain one or more hetero atoms.

Preferably, the compounds of the present invention have the structures shown herein.

The compound of the invention, in particular the ring system compound of the present invention may contain substituents other than those shown herein. By way of example, these other substituents may be one or more of: one or more halo groups, one or more 0 groups, one or more hydroxy groups, one or more amino groups, one or more sulphur containing group(s), one or more hydrocarbyl group(s)—such as an oxyhydrocarbyl group.

In general terms the ring system of the present compounds may contain a variety of non-interfering substituents. In particular, the ring system may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

Compound

The term “compound” is intended to encompass isomeric forms (such as stereoisomers and/or geometric and/or optical isomers, and mixtures thereof), chemical derivatives, mimetics, solvates and salts of the compounds.

Hydrocarbyl

In the context of the present invention, the term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In some aspects of the present invention, one or more hydrocarbyl groups is independently selected from optionally substituted alkyl group, optionally substituted haloalkyl group, aryl group, alkylaryl group, alkylarylalkyl group, and an alkene group.

In some aspects of the present invention, one or more hydrocarbyl groups is independently selected from C₁-C₁₀ alkyl group, such as C₁-C₆ alkyl group, and C₁-C₃ alkyl group. Typical alkyl groups include C₁ alkyl, C₂ alkyl, C₃ alkyl, C₄ alkyl, C₅ alkyl, C₇ alkyl, and C₈ alkyl.

For some aspects, preferably, the hydrocarbyl group comprises a carbonyl group. More preferably, the carbonyl group has the formula COOH.

For some aspects, preferably, the hydrocarbyl group comprises an amide group. More preferably, the amide group has the formula CH₂C(O)NH₂.

More preferably, the R2 group of the compound of Formula (I) is a hydrocarbyl group comprising an amide group.

More preferably, the R1 group of the compound of Formula (I) is a hydrocarbyl group comprising a carbonyl group.

Most preferably, the R2 group of the compound of Formula (I) is a hydrocarbyl group comprising an amide group of the formula CH₂C(O)NH₂.

Most preferably, the R1 group of the compound of Formula (I) is a hydrocarbyl group comprising a carbonyl group of the formula COOH.

In some aspects of the present invention, one or more hydrocarbyl groups may be independently selected from one or more oxyhydrocarbyl groups.

Oxyhydrocarbyl

The term “oxyhydrocarbyl” group as used herein means a group comprising at least C, H and O and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one embodiment of the present invention, the oxyhydrocarbyl group is a oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Stereo and Geometric Isomers

Some of the compounds/agents of the present invention may exist as stereoisomers and/or geometric isomers—e.g. they may possess one or more asymmetric and/or geometric centres and so may exist in two or more stereoisomeric and/or geometric forms. The present invention contemplates the use of all of the individual stereoisomers and geometric isomers of those compounds, and mixtures thereof. The terms used in the claims encompass these forms, provided said forms retain the appropriate functional activity (though not necessarily to the same degree).

Solvates

The present invention also includes the use of solvate forms of the compounds/agents of the present invention. The terms used in the claims encompass these forms.

Pro-Drug

As indicated, the present invention also includes the use of pro-drug forms of the compounds/agents of the present invention. The terms used in the claims encompass these forms. Examples of prodrugs include entities that have certain protected group(s) and which may not possess pharmacological activity as such, but may, in certain instances, be administered (such as orally or parenterally) and thereafter metabolised in the body to form the compounds of the present invention which are pharmacologically active.

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the compounds. Such prodrugs are also included within the scope of the invention.

An example of a prodrug according to the present invention is:

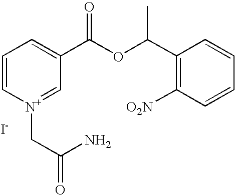

This prodrug requires light for activation.

Mimetic

In one embodiment of the present invention, the compound/agent may be a mimetic.

As used herein, the term “mimetic” relates to any chemical which includes, but is not limited to, a peptide, polypeptide, antibody or other organic chemical which has the same qualitative activity or effect as a reference agent.

In a preferred embodiment, the compound is a mimetic of nicotinic group of NAADP, wherein said NAADP has the formula (II)

Chemical Derivative

In one embodiment of the present invention, the compound/agent may be a derivative.

The term “derivative” as used herein includes chemical modification of a compound/agent. Illustrative of such chemical modifications would be replacement of hydrogen by a halo group, an alkyl group, an acyl group or an amino group.

Chemical Modification

In one embodiment of the present invention, the compound/agent may be a chemically modified compound/agent.

The chemical modification of a compound/agent may either enhance or reduce hydrogen bonding interaction, charge interaction, hydrophobic interaction, van der Waals interaction or dipole interaction between the agent and the target.

General Assay Techniques

In one aspect, the identified compounds/agents according to the present invention may act as a model (for example, a template) for the development of other compounds. The compounds/agents employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through put screen.

Techniques for drug screening may be based on the method described in Geysen, European Patent Application 84/03564, published on Sep. 13, 1984. In summary, large numbers of different small peptide test compounds are synthesised on a solid substrate, such as plastic pins or some other surface. The peptide test compounds are reacted with a suitable compound or fragment thereof and washed. Bound entities are then detected—such as by appropriately adapting methods well known in the art. A purified compound can also be coated directly onto plates for use in a drug screening techniques. Alternatively, non-neutralizing antibodies can be used to capture the peptide and immobilize it on a solid support.

This invention also contemplates the use of competitive drug screening assays in which neutralizing antibodies capable of binding a compound/agent specifically compete with a test compound for binding to a compound according to the present invention.

Another technique for screening provides for high throughput screening (HTS) of compounds/agents having suitable binding affinity to the substances and is based upon the method described in detail in WO 84/03564.

It is expected that the assay methods of the present invention will be suitable for both small and large-scale screening of test compounds as well as in quantitative assays.

Host Cells

The term “host cell”—in relation to the present invention includes any cell that could comprise the target—such as the NAADP receptor—for the compound/agent of the present invention.

Thus, host cells may be transformed or transfected with a polynucleotide that is or expresses the target of the present invention. Preferably said polynucleotide is carried in a vector for the replication and expression of polynucleotides that are to be the target or are to express the target. The cells will be chosen to be compatible with the said vector and may for example be prokaryotic (for example bacterial), fungal, yeast or plant cells.

The gram negative bacterium E. coli is widely used as a host for heterologous gene expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide encoding the polypeptide of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus oryzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Organism

The term “organism” in relation to the present invention includes any organism that could comprise the target according to the present invention and/or products obtained therefrom. Examples of organisms may include a fungus, yeast or a plant.

The term “transgenic organism” in relation to the present invention includes any organism that comprises the target according to the present invention and/or products obtained.

Transformation of Host Cells/Host Organisms

As indicated earlier, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence into a construct designed for expression in yeast. Several types of constructs used for heterologous expression have been developed. The constructs contain a promoter active in yeast fused to the nucleotide sequence, usually a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, e.g. G418.

Another host organism is a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

Thus, the present invention also provides a method of transforming a host cell with a nucleotide sequence that is to be the target or is to express the target. Host cells transformed with the nucleotide sequence may be cultured under conditions suitable for the expression of the encoded protein. The protein produced by a recombinant cell may be displayed on the surface of the cell. If desired, and as will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

Expression Vectors

The nucleotide sequence for use as the target or for expressing the target can be incorporated into a recombinant replicable vector.

The vector may be used to replicate and express the nucleotide sequence in and/or from a compatible host cell. Expression may be controlled using control sequences which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters described above.

The protein produced by a host recombinant cell by expression of the nucleotide sequence may be secreted or may be contained intracellularly depending on the sequence and/or the vector used. The coding sequences can be designed with signal sequences which direct secretion of the substance coding sequences through a particular prokaryotic or eukaryotic cell membrane.

Fusion Proteins

The target amino acid sequence may be produced as a fusion protein, for example to aid in extraction and purification. Examples of fusion protein partners include glutathione-S-transferase (GST), 6×His, GAL4 (DNA binding and/or transcriptional activation domains) and (-galactosidase. It may also be convenient to include a proteolytic cleavage site between the fusion protein partner and the protein sequence of interest to allow removal of fusion protein sequences. Preferably the fusion protein will not hinder the activity of the target.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

Reporters

A wide variety of reporters may be used in the assay methods (as well as screens) of the present invention with preferred reporters providing conveniently detectable signals (e.g. by spectroscopy). By way of example, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Assay Method

In a further aspect, the present invention relates to an assay method for identifying an agent that modulates intracellular calcium release comprising the steps of: (a) providing an agent; (b) providing an NAADP receptor; (c) contacting said agent with an NAADP receptor; and (d) measuring the level of intracellular calcium release; wherein a difference between (i) the level of intracellular calcium release in the presence of the agent; and (ii) the level of intracellular calcium release in the absence of the agent is indicative of an agent that modulates intracellular calcium release and may be useful in one or more of: modulating the release of intracellular calcium from a store controlled by nicotinic acid adenine dinucleotide phosphate; modulating calcium spikes in mammalian cells; treating diseases in one or more of brain, heart, pancreatic cells (e.g. pancreatic acinar and pancreatic beta cells), immune cells, T-cells, haemopoietic cells including phagocytes; treating diseases in one or more of brain, heart, pancreatic cells (e.g. pancreatic acinar and pancreatic beta cells), immune cells, T-cells, haemopoietic cells including phagocytes by modulating the release of intracellular calcium from a store controlled by nicotinic acid adenine dinucleotide phosphate; and treating diseases in one or more of brain, heart, pancreatic cells (e.g. pancreatic acinar and pancreatic beta cells), immune cells, T-cells, haemopoietic cells including phagocytes by modulating calcium spikes in mammalian cells.

Agent

As described herein, the assay methods of the present invention may be used to identify one or more agents that modulates intracellular calcium release.

As used herein, the term “agent” may refer to a single entity or a combination of entities.

The agent may be an organic compound or other chemical. The agent may be a compound, which is obtainable from or produced by any suitable source, whether natural or artificial.

The agent may be an amino acid molecule, a polypeptide, or a chemical derivative thereof, or a combination thereof.

The agent may even be a polynucleotide molecule—which may be a sense or an anti-sense molecule.

The agent may even be an antibody or a part or parts thereof.

The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, a peptide synthesised synthetically (such as, by way of example, either using a peptide synthesizer or by recombinant techniques) or combinations thereof, a recombinant agent, an antibody, a natural or a non-natural agent, a fusion protein or equivalent thereof and mutants, derivatives or combinations thereof.

The agent may be an organic compound. Typically the organic compounds may comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The agent may comprise at least one cyclic group. The cyclic group may be a polycyclic group, such as a non-fused polycyclic group. The agent may comprise at least one of said cyclic groups linked to another hydrocarbyl group.

The agent may contain halo groups.

The agent may contain one or more of alkyl, alkoxy, alkenyl, alkylene and alkenylene groups—which may be unbranched- or branched-chain.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19.

The agent may be capable of displaying other therapeutic properties.

The agent may be used in combination with one or more other pharmaceutically active agents.

In a highly preferred aspect, the agent is cell permeable; has a relative molecular mass of less than about 500; is a mimetic of the nicotinic group of NAADP, wherein said NAADP has the formula:

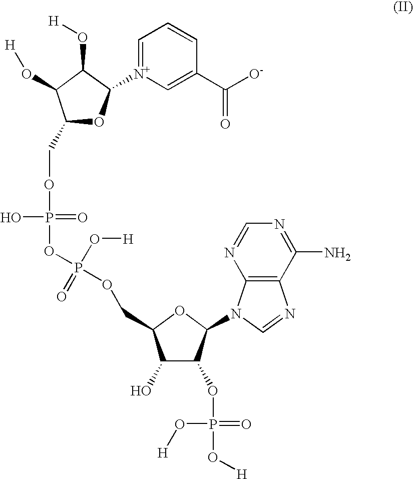

If combinations of active agents are administered, then they may be administered simultaneously, separately or sequentially.

Pharmaceutical Salts

The compounds and/or agents of the present invention may be administered as pharmaceutically acceptable salts. Typically, a pharmaceutically acceptable salt may be readily prepared by using a desired acid or base, as appropriate. The salt may precipitate from solution and be collected by filtration or may be recovered by evaporation of the solvent.

Pharmaceutically-acceptable salts are well known to those skilled in the art, and for example include those mentioned by Berge et al, in J. Pharm. Sci. 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

The compounds and/or agents of the present invention may exist in polymorphic form.

In addition, the compounds and/or agents of the present invention may contain one or more asymmetric carbon atoms and therefore exists in two or more stereoisomeric forms. Where a compound and/or agent contains an alkenyl or alkenylene group, cis (E) and trans (Z) isomerism may also occur. The present invention includes the individual stereoisomers of the compound and/or agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the compound and/or agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also includes all suitable isotopic variations of the compound and/or agent or a pharmaceutically acceptable salt thereof. An isotopic variation of a compound and/or agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the compound and/or agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the compound and/or agent and pharmaceutically acceptable salts thereof, for example, those in which a radioactive isotope such as ³H or ¹⁴C is incorporated, are useful in drug and/or substrate tissue distribution studies. Tritiated, i.e., 3H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compound and/or agent of the present invention and pharmaceutically acceptable salts thereof of this invention can generally be prepared by conventional procedures using appropriate isotopic variations of suitable reagents.

The present invention also includes (wherever appropriate) the use of zwitterionic forms of the compounds and/or agents of the present invention.

The terms used in the claims encompass one or more of the forms just mentioned.

Formulation

The component(s) of the present invention may be formulated into a pharmaceutical composition, such as by mixing with one or more of a suitable carrier, diluent or excipient, by using techniques that are known in the art.

Pharmaceutical Compositions

The present invention provides a pharmaceutical composition comprising a therapeutically effective amount of one or more compounds and/or agents of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Examples of suitable carriers include lactose, starch, glucose, methyl cellulose, magnesium stearate, mannitol, sorbitol and the like. Examples of suitable diluents include ethanol, glycerol and water.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be administered by a number of routes.

Where the composition is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, one or more compounds and/or agents may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The pharmaceutical composition may comprise one or more additional pharmaceutically active compounds and/or agents.

Chemical Synthesis Methods

The compounds and/or agents of the invention may be prepared by chemical synthesis techniques.

It will be apparent to those skilled in the art that sensitive functional groups may need to be protected and deprotected during synthesis of a compound and/or agent of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be epimerised, for example if a base is used in a reaction with a substrate having an having an optical centre comprising a base-sensitive group. It should be possible to circumvent potential problems such as this by choice of reaction sequence, conditions, reagents, protection/deprotection regimes, etc. as is well-known in the art.

The compounds/agents and salts of the invention may be separated and purified by conventional methods.

Therapy

As with the term “treatment”, the term “therapy” includes curative effects, alleviation effects, and prophylactic effects.

Preferably, the term therapy includes at least curative treatment and/or palliative treatment.

The therapy may be on humans or animals.

In one aspect, the present invention relates to the use of compound (I) wherein: R1 comprises a carbonyl group; R2 is a hydrocarbyl group; optionally wherein said ring is further substituted; or a pharmaceutically acceptable salt thereof in the manufacture of a medicament for the treatment of a disease.

Combination Pharmaceutical

The compound/agents of the present invention may be used in combination with one or more other active agents, such as one or more other pharmaceutically active agents.

Administration

The components of the present invention may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the composition can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical composition is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compounds/agents may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the composition comprises more than one compound/agent, it is to be understood that not all of the components of the pharmaceutical need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds/agents can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) of the present invention can be formulated as a suitable ointment containing the one or more active compounds/agents suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

In a preferred embodiment of the invention, the pharmaceutical composition is administered orally.

Dose Levels

Typically, a physician will determine the actual dosage which will be most suitable for an individual subject. The specific dose level and frequency of dosage for any particular patient may be varied and will depend upon a variety of factors including the activity of the specific one or more compounds/agents employed, the metabolic stability and length of action of that compound/agent, the age, body weight, general health, sex, diet, mode and time of administration, rate of excretion, drug combination, the severity of the particular condition, and the individual undergoing therapy.

The routes of administration and dosages described are intended only as a guide since a skilled practitioner will be able to determine readily the optimum route of administration and dosage for any particular patient depending on, for example, the age, weight and condition of the patient.

Other Therapies

It is also to be understood that the compounds/agents/compositions of the present invention may have other important medical implications.

For example, the compounds, compositions or agents of the present invention may be useful in the treatment of the disorders listed in WO-A-99/52890.

In addition, or in the alternative, the compounds, compositions or agents of the present invention may be useful in the treatment of the disorders listed in WO-A-98/05635. For ease of reference, part of that list is now provided: diabetes including Type II diabetes, obesity, cancer, inflammation or inflammatory disease, dermatological disorders, fever, cardiovascular effects, hemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compounds, compositions or agents of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilizing specific cell types to sites of injury or infection); hemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In a preferred aspect, the condition or disease is selected from the list consisting of:
modulating the release of intracellular calcium from a store controlled by nicotinic acid adenine dinucleotide phosphate;
modulating calcium spikes in mammalian cells;
   treating diseases in one or more of brain, heart, pancreatic cells (e.g. pancreatic acinar and pancreatic beta cells), immune cells, T-cells, haemopoietic cells including phagocytes;
   treating diseases in one or more of brain, heart, pancreatic cells (e.g. pancreatic acinar and pancreatic beta cells), immune cells, T-cells, haemopoietic cells including phagocytes by modulating the release of intracellular calcium from a store controlled by nicotinic acid adenine dinucleotide phosphate; and
   treating diseases in one or more of brain, heart, pancreatic cells (e.g. pancreatic acinar and pancreatic beta cells), immune cells, T-cells, haemopoietic cells including phagocytes by modulating calcium spikes in mammalian cells.

In a preferred aspect, the condition or disease is selected from the list consisting of:
treating an autoimmune disease (such as thyroiditis, insulitis, multiple sclerosis, invectitis, orchitis, myasthenia gravis, rhematoid arthritis or lupus erythematosis) or graft rejection, or Type II diabetes, or cardiac arrhythmia, or treating or preventing an immune disorder in a human or animal.
   General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The invention will now be further described by way of Examples, which are meant to serve to assist one of ordinary skill in the art in carrying out the invention and are not intended in any way to limit the scope of the invention.

### EXAMPLES

Material and Methods

Thin layer chromatography (TLC) was performed on precoated plates (Merck TLC aluminum sheets silica 60 F254) with eluants as indicated. The compounds were detected using a UV lamp at 254 nm. Flash chromatography was carried out using Sorbisil c60 silica gel.

¹H and ¹³C NMR spectra were recorded on either a JEOL GX270 or 400 spectrometer. Unless otherwise stated chemical shifts were measured in parts per million (ppm) relative to residual protonated solvent.

Melting points were determined using a Reichert Jung Thermo Galen Kofler block and are uncorrected. Mass spectra were recorded at the University of Bath Mass Spectrometry Service using positive and negative fast atom bombardment (FAB) with 3-nitro benzyl alcohol (NBA) as a matrix carrier.

Intracellular Calcium Measurements

Subcellular fractions: calcium concentrations are measured with fluo-3 (3 μM) at physiological temperatures, using cell homogenates or subcellular fractions in a fluorimeter (Perkin-Elmer LS-50B) at 506 nm excitation and 526 nm emission.
Intact cells: Injected or hydrolysed ester derivatives of calcium sensitive dye are imaged in intact cells by laser-scanning confocal microscopy (TCS NT, Leica) Images were processed with the software NIH Image to create a self-ratio by dividing each image (F) by an image acquired before stimulation (Fo). 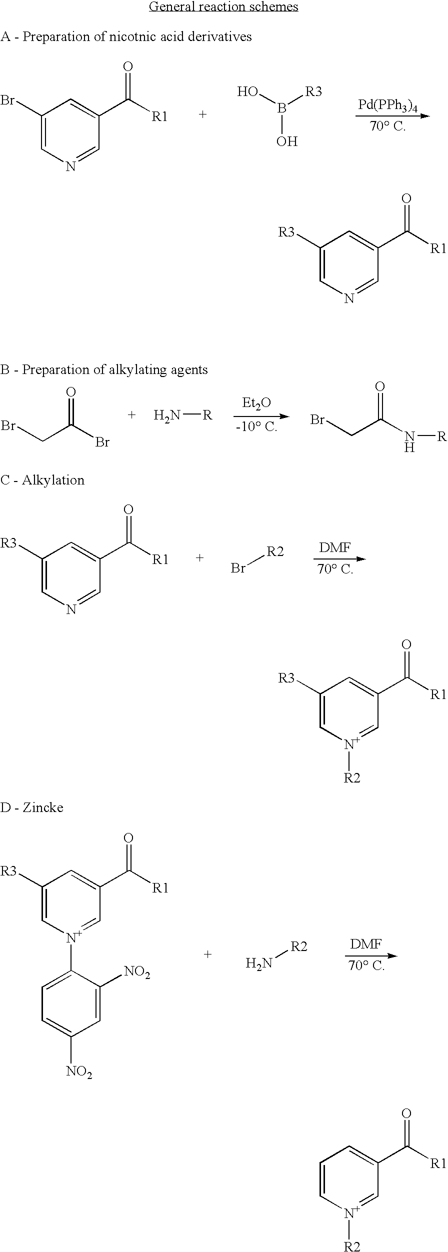
   wherein R1, R2 and R3 are as defined herein.
   I Preparation of Nicotinic Acid Derivatives

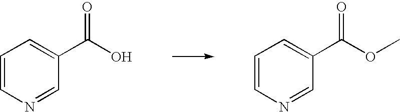

Nicotinic acid (5 g, 40.6 mmol) in methanol (85 mL) and concentrated sulfuric acid (17 mL) was refluxed for 14 hours. Water (50 mL) was then added and the aqueous solution was then neutralised with saturated NaHCO₃ (˜300 mL), extracted with chloroform (3×50 mL), dried (Na₂SO₄), filtered and evaporated under reduced pressure to afford a pale yellow liquid which crystallize on standing as a white solid (4.8 g, 88%) m.p. 34-36° C. which showed δ_{H} (400 MHz, CDCl₃) 9.23 (1H, d, J 1.6, H2), 9.78 (1H, dd, J 4.9 and 1.6, H4), 8.29 (1H, m, H6), 7.40 (1H, m, H5) and 3.96 (3H, s, CH₃).

### Example 1

### 5-Bromo-nicotinic acid methyl ester

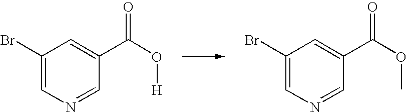

5-Bromo-nicotinic acid (300 mg, 1.5 mmol) was added methanol (6 ml) and concentrated H₂SO₄ (1.1 ml). This reaction mixture was then heated at reflux for overnight. After cooling down, about 5 ml of concentrated NaHCO₃ solution was added to adjust the PH to 7-8. The product was then extracted with DCM (20 ml). Removal of the solvent gave the product as white solid in 81% yield.

IR: 3450, 3047, 1722, 1577, 1419, 1311, 1107, 1016, 955, 764, 688; ¹H NMR (270 MHz, CDCl₃): 9.12 (s, 1H, H-2), 8.85 (m, 1H, H-6), 8.44 (m, 1H, H-4), 3.99 (s, 3H, CH₃); ¹³C NMR (100.4 MHz, CDCl₃): 164.4 (CO), 154.4 (C2), 148.7 (C6), 139.6 (C4), 127.4 (C3), 120.7 (C5), 52.9 (CH₃); MS: m/z (FAB⁺) 216.2 (M⁺).

### Nicotinic acid 1-(2-nitro-phenyl)-ether ester

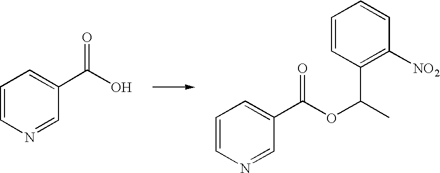

To a solution of 1-(2-nitrophenyl)ethanol (618 mg, 3.7 mmol) in dry dichloromethane (18 mL) was added nicotinic acid (500 mg, 4.1 mmol), dicyclohexylcarbodiimide (838 mg, 4.1 mmol) and DMAP (45 mg, 0.37 mmol). The reaction was stirred at room temperature overnight after which the urea was filtered. The filtrate was diluted in chloroform (20 mL), extracted with saturated NaHCO₃ (15 mL) and brine (15 mL). The combined extracts were dried (Na₂SO₄), filtered and evaporated under reduced pressure. The residue was purified on silica gel (hexane:EtOAc-7:3) to afford the desired ester as a pale yellow oil (910 mg, 81%) which showed δ_{H} (270 MHz, CDCl₃) 9.16 (1H, d, J 1.5, H2), 8.70 (1H, dd, J 4.8 and 1.8, H6), 8.21 (1H, m, H), 7.88 (1H, dd, J 8.4 and 1.1, CH), 7.65 (1H, dd, J 8.1 and 1.8, H4), 7.56 (1H, dt, J 7.5 and 1.1, CH), 7.40-7.30 (2H, m, 2×CH), 6.51 (1H, q, J 6.4, CH) and 1.71 (3H, t, J 6.4, CH₃). δ_{C} (65 MHz, CDCl₃) 164.3 (C═O), 153.2, 150.7 (both CH), 137.3 (C), 137.0, 133.6, 128.5, 126.9 (all CH), 125.6 (C), 124.4, 123.3 (both CH), 69.2 (CH) and 21.9 (CH₃). 1 C hidden. m/z [FAB⁺] 273.1 (M⁺+H, 100%) [found 273.0871 M⁺+H. C₁₄H₁₃N₂O₄ requires 273.0873].

### 5-(4-Methoxy-phenyl)-nicotinic acid methyl ester

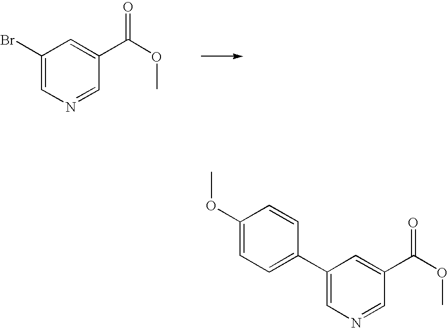

5-Bromo-nicotinic acid methyl ester (153 mg, 0.7 mmol) was added toluene (1.4 ml) and 2M Na₂CO₃ (0.7 ml), To this suspension, Pd(PPh₃)₄ (24 mg) was added under N₂. Then a solution of p-methoxyphenylboronic acid (127.6 mg) in methanol (0.35 ml) was added. This reaction mixture was vigorously stirred and heated to 80° C. for 6 h. After cooling, the crude product was partitioned between DCM (7 ml) and 2M Na₂CO₃ (3.5 ml+0.35 ml NH3).

Removal of the organic layer gave light yellow crude product, which was then purified by silica gel column, eluted with DCM. White solid was given in 41% yield.

IR: 3036, 2956, 2844, 1716, 1609, 1518, 1443, 1427, 1309, 1111, 1026, 841; ¹HNMR (270 MHz, CDCl₃): 9.13 (d, J=1.9, 1H, H-2), 8.95 (d, J=2.4, 1H, H-6), 8.43 (m, 1H, H-4), 7.55 (d, J=5.4, 2H, ArH-2 and 6), 7.03 (d, J=5.4, 2H, ArH-3 and 5), 3.97 (s, 3H, CH₃), 3.86 (s, 3H, CH₃); ¹³C NMR (100.4 MHz, CDCl₃): 165.6 (CO), 160.1 (ArC4), 151.4 (C6), 148.7 (C2), 136.1 (C4), 134.7 (C5), 129.0 (ArC1), 128.3 (ArC2 and C6), 125.9 (C3), 114.7 (ArC3 and C5), 55.6 (OCH₃), 52.7 (CH₃—CO); MS: m/z (FAB⁺) 244.3 (M⁺+1).

Nicotinic acid (100 mg, 0.812 mmol) and organic halide (0.812 mmol) were mixed in dry DMF (1.5 mL) and stirred at 50° C. in the dark overnight after which time DMF was evaporated under reduced pressure. The resulting product was then recrystallized to afford the corresponding pyridinium salt.

II Preparation of (Bromoacetylamide) Alkylating Agents.

Amine (3 ml, 24 mmol) was added dropwise to a stirring solution of 2-bromoacetylbromide (10 ml, 12 mmol) at −10° C., such that the temperature did not rise above 0° C. When the reaction was complete, cold water was added to dissolve the precipitated salt. The organic layer was separated and washed successively with aqueous acetic acid (20 ml, 1M), aqueous NaOH (20 ml, 1M) and finally with saturated brine. After removal of the ether, the crude product was used for alkylation directly without further purification. Analytical data was given by recrystallization or flash chromatography.

### 2-Bromo-N,N-diethyl-acetamide (2)

Colorless oil was given after working up. ¹H NMR (CDCl₃): δ 3.73 (s, 2H, —CH₂CO) 3.25 (q, 4H, J=7.5 Hz, —CH₂ CH₃), 1.24(t, 3H, J=7.5 Hz, —CH₃), 1.03(t, 3H, J=7.5 Hz, —CH₃). Yield 60%. Data is identical to the literature [1].

### 2-Bromo-N-butyl-acetamide (3)

This product is a solid at room temperature. Mp: 30° C. ¹HNMR (CDCl₃): δ 6.65 (brs, 1H, NH), 3.81 (s, 2H, —CH2CO), 3.19 (m, 2H, N—CH2), 1.41(m, 2H, —CH2 CH2), 1.25 (m, 2H, —CH2 CH3), 0.78 (t, 3H, —CH3). Yield 76%. Data is identical to the literature [1].

### 2-Bromo-N-phenyl-acetamide (4)

This product is a yellow solid. Mp: 128-131. IR: 3296, 3203, 3146, 3099, 1657, 1618, 1555, 1486, 1445, 1335, 1111; ¹HNMR (CDCl₃): 8.13 (brs, 1H, NH) 7.52 (d, 2h, J=2.7 Hz, ArH2, 6), 7.34 (m, 2H, ArH3, 5), 7.17(m, 1H, ArH4), 4.00(s, 2H, —CH₂CO). MS: m/Z (FAB⁺) 213.9 (M⁺+1).

### 2-Bromo-N-cyclohexyl-acetamide (5)

This product is a white fine powder. Yield 90%. Mp: 106-108. IR: 3284, 3072, 2937, 2919, 2853, 1645, 1551, 1446, 1328, 1210, 1155, 1089; ¹H NMR (CDCl₃): 6.33 (brs, 1H, NH), 3.84(s, 2H, —CH₂CO), 3.74(m, 1H, N—CH), 1.10-1.92 (m, 10H, ring-H). ¹³C NMR (CDCl₃): 164.7 (CO), 49.6 (CH), 33.4 (2CH₂), 30.2 (CH₂—Br), 26.1 (2CH₂), 25.3 (CH₂). MS: m/z (FAB⁺) 220 (M⁺+1).

### 2-Bromo-N-(4-ethyl-phenyl)-acetamide (6)

This product is yellow solid. Mp: 131-132. ¹H NMR (CDCl₃): 8.07 (br, 1H, NH), 7.40 (d, 2H, J=8.4 Hz, ArH), 7.15 (d, 2H, J=8.4 Hz, ArH), 4.00 (s, 2H, —CH₂CO), 2.59 (q, 2H, J=7.67 Hz, —CH₂ CH₃), 1.18 (t, 3H, J=7.67 Hz, —CH₃). MS: m/z (FAB⁺) 242.0 (M⁺+1).

### 2-Bromo-N-(2-Bromophenyl)-acetamide (7)

Crude product was subjected to silica gel column, eluted with DCM:Hexane 10:1. Yellow solid was given in 52% yield.

Mp: 96-98. IR: 3184, 3107, 1653, 1609, 1591, 1542, 1472, 1422, 1315, 700; ¹H NMR (CDCl₃): 8.15 (s, br, 1H, NH), 7.77 (m, 1H, H-3), 7.43 (d, J=7.83, 1H, H-5), 7.23 (m, 2H, H-2, 3), 4.00 (s, 2H, CH₂); ¹³C NMR (CDCl₃): 164.3 (C═O), 138.1 (C-1), 130.3 (C-3), 128.2 (C-5), 123.0 (C-4), 122.6 (C-2), 118.5 (C-6); MS (FAB⁺): m/z 293 (M⁺+1).

### 2-Bromo-N,N-dibutylacetamide (8)

Mp: oil. IR: 2959, 2932, 2872, 1648, 1460, 1376, 1209, 1098; ¹H NMR (CDCl₃): 3.77 (s, 2H, CO—CH₂), 3.23 (m, 4H, 2NCH₂), 1.48 (m, 4H, 2CH₂), 1.25 (m, 4H, 2CH₂), 0.86 (m, 6H, 2CH₃); ¹³C NMR (CDCl₃): 166.3 (CO), 48.8 (CH₂), 46.2 (CH₂), 31.5 (CH₂—Br), 29.7 (CH₂), 27.0 (CH₂), 20.5 (2CH₂), 14.27 (CH₃), 14.19 (CH₃); MS m/z (FAB⁺) 250 (M⁺+1), 170 (M⁺−Br), 128 (M⁺−BrCOCH₂).

### 2-Bromo-N,N-dicyclohexylacetamide (9)

The crude product was purified by Flash chromatography (DCM:hexane 12:1). White solid was obtained in 40% yield. Analytical data was given by crystallization from DCM.

Mp: 122-124, IR: 3466, 2933, 2869, 2857, 1637, 1467, 1371, 1113, 1000; ¹H NMR (CDCl₃): 3.80 (s, 2H, CO—CH₂), 2.41 (m, 2H, 2CH), 1.17-1.86 (m, 20H, ring-H); ¹³C NMR (CDCl₃): 162.6 (CO), 56.3 (N—C), 31.0 (CH₂), 29.4 (CH₂), 29.1(Br—CH₂), 26.5-25.2 (ring-C). MS: m/z (FAB⁺) 302.0 (M⁺).

### 2-Bromo-N-decylacetamide (10)

Colourless oil was given after purified by Flash chromatography (DCM:hexane 10:1). Yield 81%.

Mp: oil, IR: 3282, 2920, 2852, 1640, 1560, 1471, 1436, 1214; ¹H NMR (CDCl₃): 6.49 (s, brs, 1H, NH), 3.86 (s, 2H, CH₂—Br), 3.25 (m, 2H, CH₂), 2.15 (s, acetone peak), 1.52 (m, 2H, CH₂), 1.24 (m, 14H, 7CH₂), 0.86 (m, 3H, CH₃); ¹³C NMR (CDCl₃): 165.4 (CO), 40.7 (CH₂—Br), 32.3 (CH₂—N), 29.7-29.9 (6CH₂), 27.2 (CH₂), 23.1 (CH₂), 14.6 (CH₃);

### 2-Bromo-N-methyl-N-phenylactamide (11)

Yellow solid was given after working up. Yield 80%. Mp: 43-45. IR: 1646, 1595, 1498, 1385, 1298, 1120, 1043, 882, 771; ¹H NMR (CDCl₃): 7.28 (m, 5H, Ar—H), 3.87 (s, 2H, CH₂), 3.29 (s, 3H, CH₃). MS: m/z (FAB⁺) 228 (M⁺+1).

### 2-Bromo-N-(3-methoxybenzyl)acetamide (12)

The crude product was subjected to silica gel column, eluted with DCM:hexane 10:1. Yellow solid was given in 72% yield.

Mp: 79-80. IR: 3289, 3048, 1641, 1543, 1489, 1441, 1296, 1200, 1158, 1049, 999, 890, 784, 697; ¹H NMR (CDCl₃): 7.24-6.83 (m, 4H, Ar—H), 4.40 (s, 2H,Br—CH₂), 3.86 (s, 2H, CO—CH₂), 3.77 (s, 3H, CH₃). ¹³C NMR (CDCl₃): 165.8 (CO), 159.9 (C—O), 139.1 (C), 130.0 (C5), 120.0 (C6), 113.6 (C2), 113.3 (C4), 55.6 (OCH₃), 44.4 (COCH₂), 29.4 (Br—CH₂); MS m/z (FAB⁺) 257.9 (M⁺+1), 178.0 (M⁺−Br).

### 2-Bromo-N,N-dipropylacetamide (13)

Yellow oil was given after working up in 65% yield.

IR: 3451, 2966, 2876, 1645, 1456, 1381, 1302, 1207, 1098, 896,744; ¹H NMR (CDCl₃): 3.81 (s, 2H, CH₂—Br), 3.24 (m, 4H, 2CH₂), 1.55 (m, 4H, 2CH₂), 0.86 (m, 6H, 2CH₃); ¹³C NMR (CDCl₃): 166.5 (CO), 50.3 (CH₂), 47.7 (CH₂), 26.2 (CH₂—Br), 22.2 (CH₂), 20.4 (CH₂), 11.2 (2CH₃). MS m/z (FAB⁺) 222 (M⁺+1).

### 2-Bromo-N,N-dioctylacetamide (14)

Crude product was purified by Flash chromatography (DCM:hexane 9:1). Yellow oil was given in 81% yield.

IR: 2925, 2855, 1649, 1460, 1376, and 1101. ¹HNMR(CDCl₃): 3.80 (s, 2H, CH₂—Br), 3.26 (m, 4H, 2CH₂), 1.54 (m, 4H, 2CH₂), 1.25 (m, 20H, CH₂), 0.84 (m, 6H, 2CH₃); ¹³C NMR (CDCl₃): 166.3 (CO), 49.1 (CH₂), 46.5 (CH₂), 27.6 (CH₂—Br), 32.2-23.0 (CH₂), 14.6 (2CH₃). Ms m/z (FAB⁺) 362 (M⁺+1).

### Example 2

### [C] N-Alkylation of nicotinic acid methyl ester

Nicotinic acid methyl ester (500 mg, 3.65 mmol), the halide (3.65 mmol) and sodium iodide (3.65 mmol) were stirred in DMF or acetonitrile in the dark at 50° C. for 24 hours. The solvent was then evaporated under reduced pressure and crystallized from methanol/ether.

### 1-[(2-Bromo-phenylcarbamoyl)-methyl]-3-methoxycarbonyl-pyridinium (25)

MP: 171-172, IR: 3225, 3173, 3057, 3015, 1743, 1687, 1596, 1549, 1307, 780, 740; ¹H NMR (DMSO): 10.97 (s, brs, 1H, NH), 9.68 (s, 1H, H-2), 9.26 (d, J=6.2, 1H, H-6), 9.11 (d, J=8.1, 1H, H-4), 8.38 (dd, J=6.2 and 8.1, 1H, H-5), 7.92 (s, 1H, H-3′), 7.32-7.50 (m, 3H, ArH), 5.78 (s, 2H, CH₂), 3.99 (s, 3H, CH₃); ¹³C NMR (DMSO): 164.1 (COOMe), 162.6 (CO—N), 150.4 (C-2), 148.1 (C-6), 146.6 (C-4), 131.7 (C—N), 129.8 (C-3′), 128.5 (C-5′), 127.3 (C-2′), 122.3 (C-5), 122.2 (C-6′), 63.2 (CH₂—CO), 54.4 (CH₃); MS: m/z (FAB⁺) 351.1 (M⁺+1).

### Example 3

### Diethylcarbamoylmethyl-3-methoxycarbonyl-pyridinium; iodide

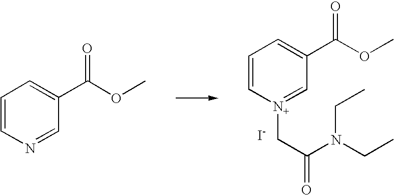

Nicotinic acid methyl ester and 2-chloro-N,N′-diethylacetamide were reacted in DMF under the standard protocol to afford a bright yellow solid (363 mg, 26%) m.p. 163-165° C. which showed δ_{H} (270 MHz, D₂O) 9.30 (1H, s, H2), 9.07 (1H, d, J 8.2, H6), 8.90 (1H, d, J 6.2, H4), 8.19 (1H, m, H5), 5.75 (2H, s, py-CH₂), 3.96 (3H, s, OCH₃), 3.41 (2H, q, J 7.2, CH₂), 3.84 (2H, q, J 7.2, CH₂), 1.23 (3H, t, J 7.2, CH₃) and 1.05 (3H, t, J 7.2, CH₃). δ_{C} (65 MHz, D₂O) 164.3 (C═O), 149.2, 147.6, 146.8 (all CH), 130.6 (C), 128.3 (CH), 62.1 (py-CH₂), 54.1 (OCH₃), 42.3, 41.9 (both CH₂), 13.2 and 12.1 (both CH₃). m/z [FAB⁺] 251.1 (M⁺, 100%) [found 251.1405 M+. C₁₃H₁₉N₂O₃I requires 251.1395]. IR (KBr) 1731 (C═O ester) and 1653 (C═O amide).

### Example 4

### 1-(2-Carbamoyl-ethyl)-3-methoxycarbonyl-pyridinium; iodide

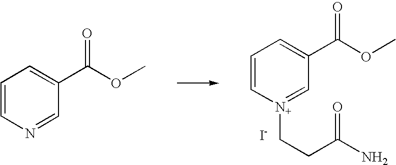

Nicotinic acid methyl ester and 3-chloropropionamide were reacted in acetonitrile under the standard protocol to afford a pale yellow solid (266 mg, 22%) m.p. 98-100° C. which showed δ_{H} (270 MHz, D₂O) 9.48 (1H, s, H2), 9.08 (1H, d, J 6.2, H6), 9.03-8.99 (1H, m, H4), 8.21-8.16 (1H, m, H5), 4.94 (2H, t, J 6.4, py-CH₂), 4.01 (3H, s, OCH₃) and 3.09 (2H, t, J 6.4, CH₂—CO). δ_{C} (65 MHz, D₂O) 173.7, 168.5 (both C═O), 148.1, 146.2 (both CH), 131.0 (C), 130.9, 128.6 (both CH), 58.2 (py-CH₂), 54.3 (OCH₃) and 35.4 (CH₂). m/z [FAB⁺] 209.1 (M⁺, 100%) [found 209.0935 M+. C₁₀H₁₃N₂O₃1 requires 209.0926]. IR (KBr) 1736 (C═O ester) and 1674 (C═O amide).

### Example 5

### 1-(3-Cyano-propyl)-3-methoxycarbonyl-pyridinium; iodide

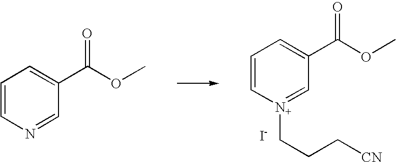

Nicotinic acid methyl ester and 4-chlorobutyronitrile were reacted in DMF under the standard protocol to afford a pale yellow solid (219 mg, 18%) m.p. 167-170° C. which showed δ_{H} (270 MHz, D₂O) 9.48 (1H, s, H2), 9.08 (1H, d, J 6.4, H6), 9.01 (1H, dt, J 8.2 and 1.5, H4), 8.20-8.17 (1H, m, H5), 4.79 (2H, t, J 7.4, py-CH₂), 3.98 (3H, s, OCH₃), 2.64 (2H, t, J 7.4, CH₂—CN) and 2.40 (2H, q, J 7.4, CH₂). 163.4 (C═O), 147.9, 146.4, 146.2 (all CH), 131.3 (C), 129.1 (CH), 60.9 (py-CH₂), 54.2 (OCH₃), 26.3 and 14.1 (both CH₂). m/z [FAB⁺] 205.0 (M⁺, 100%) [found 205.0977 M+. C₁₁H₁₄N₂O₂I requires 205.0972].

### Example 6

### 1-(2-Diethylamino-ethyl)-3-methoxycarbonyl-pyridinium; iodide

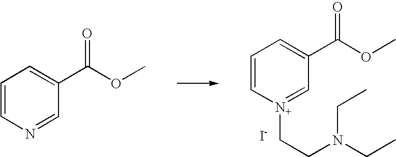

Nicotinic acid methyl ester and diethylaminoethylchloride hydrochloride were reacted in DMF under the standard protocol to afford a pale yellow solid (60 mg, 5%) m.p. 172-174° C. which showed δ_{H} (270 MHz, D₂O) 9.53 (1H, s, H2), 9.13 (1H, d, J 6.2, H6), 9.05 (1H, d, J 8.2, H4), 8.25-8.25 (1H, m, H5), 5.12 (2H, t, J 7.7, py-CH₂), 3.95 (3H, s, OCH₃), 3.82 (2H, t, J 7.7, CH₂—N), 3.30 (4H, q, J 7.4, 2×CH₂-Me) and 1.25 (6H, t, J 7.4, 2×CH₃). m/z [FAB⁺] 237.2 (M⁺, 100%) [found 237.1601 M+. C₁₃H₂₁N₂O₂I requires 237.1603].

### Example 7

### 1-(2-Cyano-ethyl)-3-methoxycarbonyl-pyridinium; iodide

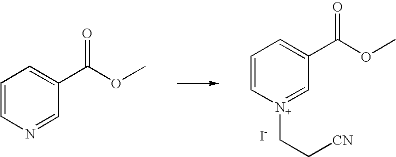

Nicotinic acid methyl ester and 3-chloropropionitrile were reacted in DMF under the standard protocol to afford a pale yellow solid (88 mg, 8%) which showed δ_{H} (270 MHz, D₂O) 9.54 (1H, s, H2), 9.11 (1H, d, J 6.2, H6), 9.05 (1H, d, J 8.2, H4), 8.25-8.20 (1H, m, H5), 4.99 (2H, t, J 6.4, py-CH₂), 3.97 (3H, s, OCH₃) and 3.29 (2H, t, J 6.4, CH₂—CN). m/z [FAB⁺] 191.0 (M⁺, 100%) [found 191.0820 M+. C₁₀H₁₁N₂O₂I requires 191.0821].

### Example 8

### 1-(1-Carbamoyl-ethyl)-3-methoxycarbonyl-pyridinium; iodide

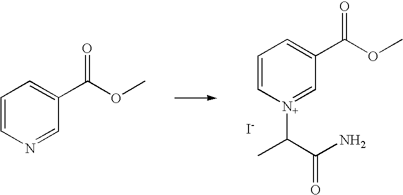

Nicotinic acid methyl ester and 2-chloro propionamide were reacted in DMF under the standard protocol to afford a yellow solid (363 mg, 26%) which showed δ_{H} (270 MHz, D₂O) 9.34 (1H, s, H2), 9.03-8.96 (2H, m, H6 and H4), 8.16-8.11 (1H, m, H5), 5.66 (1H, q, J 7.2, py-CH), 3.91 (3H, s, OCH₃) and 1.88 (3H, d, J 7.2, CH₃). δ_{C} (65 MHz, D₂O) 163.1 (C═O), 147.5, 147.0, 145.8 (all CH), 130.7 (C), 128.5 (CH), 69.2 (py-CH), 54.2 (OCH₃) and 18.2 (CH₃). m/z [FAB⁺] 209.0 (M⁺, 100%) [found 209.0929 M+. C₁₀H₁₃N₂O₃I requires 209.0927].

### 1-Cyclohexyl-3-methoxycarbonyl-5-(4-methoxy-phenyl)-pyridinium

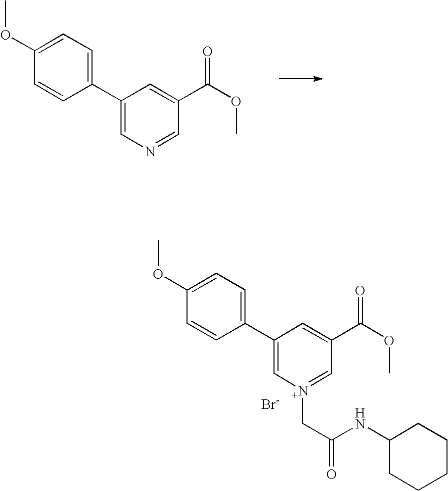

5-(4-Methoxy-phenyl)-nicotinic acid methyl ester (40) (50 mg, 0.2 mmol) was added 2-bromo-N-cyclohexylacetamide (115 mg, 0.5 mmol). To this mixture was added DMF (1 ml) under N₂ and then heated at 60° C. overnight. After removal of the solvent, the residual was dissolved in methanol (0.5 ml) and then ether (20 ml) was added to crystallize the product. Yellow oil was given (40 mg).

IR: 3436, 2929, 1738, 1652, 1557, 1450, 1346, 1260; ¹H NMR (270 MHz, CDCl₃): 9.66 (s, 1H, H-2), 9.46 (m, 1H, H-6), 9.15 (m, 1H, H-4), 8.62 (m, 1H, NH), 7.93 (m, 2H, ArH-2 and 6), 7.16 (m, 2H, ArH-3 and 5), 5.58 (s, 2H, CH₂—CO), 4.01 (s, 3H, CH₃), 3.85 (s, 3H, CH₃—CO), 1.79-1.22 (m, 11H, ring-H)

### Example 9

### Hydrolysis of N-alkylated nicotinic acid methyl ester

The corresponding nicotinic acid methyl ester (0.4 mmol) was dissolved in 48% aqueous HBr (0.2 mL) and stirred at 60° C. overnight after which it was then evaporated under reduced pressure. To the brown residue was added acetonitrile. The precipitate was filtered and washed with acetonitrile leaving the desired N-alkylated nicotinic acid.

### Example 10

### 3-Carboxy-1-diethylcarbamoylmethyl-pyridinium; iodide

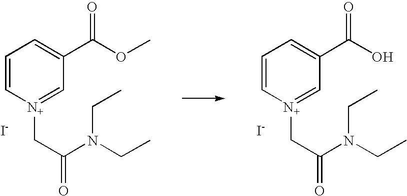

Diethylcarbamoylmethyl-3-methoxycarbonyl-pyridinium; iodide was reacted under the general protocol to afford the desired product as a white solid (104 mg, 74%) m.p. 97-100° C. which showed δ_{H} (270 MHz, D₂O) 9.19 (1H, s, H2), 8.99 (1H, d, J 8.2, H6), 8.82 (1H, d, J 6.2, H4), 8.13 (1H, m, H5), 5.71 (2H, s, py-CH₂), 3.37 (2H, q, J 7.2, CH₂), 3.32 (2H, q, J 7.2, CH₂), 1.20 (3H, t, J 7.2, CH₃) and 1.02 (3H, t, J 7.2, CH₃). δ_{C} (65 MHz, D₂O) 164.4 (C═O), 148.9, 147.7, 147.1 (all CH), 131.7 (C), 128.3 (CH), 62.1 (py-CH₂), 42.3, 41.9 (both CH₂), 13.1 and 12.1 (both CH₃). m/z [FAB⁺] 237.1 (M⁺, 100%) [found 237.1250 M+. C₁₂H₁₇N₂O₃I requires 237.1239]. IR (KBr) 3150 (OH) and 1652 (C═O amide).

### Example 11

### 1-(2-Carbamoyl-ethyl)-3-carboxy-pyridinium; iodide

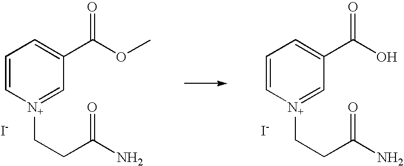

1-(2-Carbamoyl-ethyl)-3-methoxycarbonyl-pyridinium; iodide was reacted under the general protocol to afford the desired product as a white solid (120 mg, 84%) m.p. 170-173° C. which showed δ_{H} (270 MHz, D₂O) 9.38 (1H, s, H2), 9.02 (1H, d, J 6.2, H6), 8.91 (1H, d, J 7.9, H4), 8.12-8.06 (1H, m, H5), 4.87 (2H, t, J 6.2, py-CH₂) and 3.13 (2H, t, J 6.2, CH₂—CO). δ_{C} (65 MHz, D₂O) 173.7, 164.8 (both C═O), 147.8, 146.6, 146.4 (all CH), 132.4 (C), 128.5 (CH), 57.5 (py-CH₂) and 34.5 (CH₂). m/z [FAB⁺] 196.1 (M⁺, 100%) [found 196.0611 M+. C₉H₁₁N₂O₃I requires 196.0609]. IR (KBr) 3154 (OH) and 1648 (C═O amide).

### Example 12

### 3-Carboxy-1-(2-diethylamino-ethyl)-pyridinium; iodide

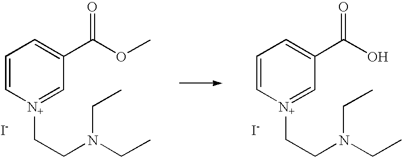

1-(2-Diethylamino-ethyl)-3-methoxycarbonyl-pyridinium; iodide was reacted under the general protocol to afford the desired product as a white solid (41 mg, 84%) m.p. 210-212° C. which showed δ_{H} (270 MHz, D₂O) 9.60 (1H, s, H2), 9.06 (1H, d, J 6.2, H6), 8.97 (1H, d, J 8.2, H4), 8.20-8.15 (1H, m, H5), 5.11 (2H, t, J 7.7, py-CH₂), 3.83 (2H, t, J 7.7, CH₂—N), 3.33 (4H, q, J 7.4, 2×CH₂-Me) and 1.26 (6H, t, J 7.4, 2×CH₃). δ_{C} (65 MHz, D₂O) 164.8 (C═O), 147.3, 146.4, 146.4 (all CH), 132.4 (C), 129.2 (CH), 55.6 (py-CH₂), 50.3, 48.2 (both CH₂) and 34.5 (CH₃).

### Example 13

### [D] Alkylation of Nicotinic Acid

Nicotinic acid (100 mg, 0.812 mmol) and organic halide (0.812 mmol) were mixed in dry DMF (1.5 mL) and stirred at 50° C. in the dark overnight after which time DMF was evaporated under reduced pressure. The resulting product was then recrystallized to afford the corresponding pyridinium salt.

### 3-Carboxy-1-diethylcarbamoylmethyl-pyridinium (15)

Yellow oil was obtained after crystallization from methanol/ether. Yield 21%.

IR (KBr) νₘₐₓ 3429, 3072, 2981, 2934, 1729, 1659, 1219, 1135, 1036, 811, 748 cm⁻¹ ¹H NMR (D₂O): 9.06 (d, 1H, J=7.5 Hz, H2), 8.87 (m, 1H, H6), 8.74 (m, 1H, H4), 8.03 (m, 1H, H5), 5.68 (s, 2H, —CH₂CO), 3.33 (q, 4H, J=7.5 Hz, —CH₂ CH₃), 1.19 (t, 3H, J=7.5 Hz, —CH₃), 1.00 (t, 3H, J=7.5 Hz, —CH₃). MS: m/z (FAB⁺) 237 (M⁺+1).

### 1-Butylcarbamoylmethyl-3-carboxy-pyridinium (16)

Mp: 190-194. IR (KBr) νₘₐₓ: 3235, 3078, 2958, 2931, 2873, 2750, 2494, 1727, 1672, 1558, 1394, 1351, 1131, 848 cm⁻¹; ¹H NMR (D₂O): 9.25 (s, 1H, H2), 8.96 (d, 1H, J=5.3 Hz, H6), 8.85 (d, 1H, J=5.9 Hz, H4), 8.13 (m, 1H, H5), 5.43 (s, 2H, —CH₂CO), 3.16 (m, 2H, N—CH₂), 1.40 (m, 2H, —CH₂ CH₂), 1.20 (m, 2H, —CH₂ CH₃), 0.77 (t, 3H, —CH₃). ¹³C NMR (D₂O): 167.9 (CO—O), 165.2 (CO—N), 148.1 (C3), 147.0 (C6), 146.7 (C2 and C4), 128.1 (C5), 62.2 (CH₂—N⁺), 39.9 (CH₂), 30.5 (CH₂), 19.7 (CH₂), 13.2 (CH₃); MS: m/z (FAB⁺) 237.0 (M⁺+1).

### 3-Carboxyl-1-phenylcarbamoylmethyl-pyridinium (17)

Mp: 148-149. IR (KBr) νₘₐₓ3393, 3277, 3080, 3019, 1699, 1637, 1599, 1558, 1498, 1446, 1350, 1311, 1258, 737, 690 cm⁻¹; ¹H NMR (D₂O): 9.18 (s, 1H, H2), 8.90 (d, 1H, J=6.6 Hz, H6), 8.83 (d, 1H, J=5.4 Hz, H4), 8.07 (m, 1H, H5), 7.25 (m, 5H, ArH), 5.59 (s, 2H, —CH₂CO). ¹³C NMR (D₂O): 163.8 (CO—O), 163.5 (CO—N), 148.2 (C6), 147.9 (C2 and C4), 146.2 (C3), 127.8 (C5), 138.9 (ArC1), 129.6 (ArC3 and C5), 119.8 (ArC2 and C6), 124.6 (ArC4); MS: m/z (FAB⁺) 257.1 (M⁺+1).

### 3-Carboxy-1-cyclohexylcarbamoylmethyl-pyridinium (18)

Mp: 188-192. IR (KBr) νₘₐₓ: 3244, 3073, 2931, 1709, 1679, 1530, 1389, 1240, 1210, 1120, 837 cm^{-1 1}H NMR (D₂O) 9.32 (s, 1H, H2), 9.00 (d, 1H, J=5.2 Hz, H6), 8.84 (d, 1H, J=4.5 Hz, H4), 8.23(m, 1H, H5), 5.50 (s, 2H, —CH₂CO), 3.65 (m, 1H, N—CH), 1.87-1.25 (m, 11H, ring-H). ¹³C NMR (D₂O): 163.6 (CO—O), 163.5 (CO—N), 149.7 (C6), 147.9 (C2), 146.4 (C4), 131.0 (C3), 128.3 (C5), 62.5 (CH₂—CO), 39.7 (CH), 33.1 (2CH₂), 25.9 (CH₂), 25.2 (2CH₂). MS: m/Z (FAB⁺) 263.3 (M⁺+1).

### 3-Carboxy-1-[(4-ethyl-phenylcarbamoyl)-methyl]-pyridinium (19)

Mp: 210-214. IR (KBr) νₘₐₓ: 3424, 3243, 3190, 3063, 2962, 2930, 1717, 1697, 1608, 1543, 1392, 1257, 1125, 834, 670 cm⁻¹. ¹H NMR (D₂O): 9.29 (s, 3.52, H2), 8.98 (d, 1H, J=8.7, H6), 8.91 (d, 1H, J=6.5, H4), 8.14 (m, 1H, H5), 7.28 (d, 2H, J=8.1 Hz, ArH2, 6), 7.18 (d, 2H, J=8.1 Hz, ArH3, 5), 5.60 (s, 2H, —CH₂CO), 2.49 (q, 2H, J=7.67 Hz, —CH₂ CH₃), 1.06 (t, 3H, J=7.67 Hz, —CH₃). ¹³C NMR (D₂O): 163.6 (CO—O), 163.4 (CO—N), 149.9 (C6), 148.3 (C2), 146.7 (C4), 140.0 (C3), 136.5 (ArC1), 131.1 (ArC4), 128.4 (C5), 128.8 (ArC3 and C5), 119.8 (ArC2 and C6), 63.1 (CH₂—CO), 28.5 (CH₂), 16.5 (CH₃). MS: m/z (FAB⁺) 285.1 (M⁺+1).

### 3-Carboxy-1-[(2-bromo-phenylcarbamoyl)-methyl]-pyridinium (20)

Mp: 216-218. IR: 3177, 3055, 2743, 1727, 1687, 1608, 1588, 1477, 1253, 1130, 850; ¹H NMR (D₂O): 9.36 (s, 1H, H-2), 9.04 (d, J=8.1, 1H, H-6), 8.99 (d, J=5.6, 1H, H-4), 8.23 (m, 1H, H-5), 7.68 (s, 1H, H-3′), 7.31 (m, 3H, H-4′, 5′, 6′), 5.70 (s, 2H, CH₂—CO), ¹³C NMR (DMSO) 163.5 (CO—OH), 163.0 (CO—NH), 147.5 (C-2), 145 (C-6), 139.3 (C-3), 131.1 (C-4), 130.0 (ArC1), 127.8 (C-5), 126.6 (ArC3), 121.6 (ArC2), 120.9 (ArC4), 116.0 (ArC6); MS m/z (FAB⁺) 335.1 (M⁺+1).

### 3-Carboxy-1-dibutylcarbamoylmethyl-pyridinium (21)

MP: Not crystal, IR: 2924, 2854, 1720, 1459, 1377, 1215; ¹H NMR (D₂O) 9.26 (s, 1H, H-2), 9.10 (m, 1H, H-6), 8.90 (m, 1H, H-4), 8.24 (m, 1H, H-5), 5.82 (s, 2H, COCH₂), 3.44 (m, 4H, 2CH₂), 1.72 (m, 2H, CH₂), 1.54 (m, 2H, CH₂), 1.40 (m, 2H, CH₂), 1.30 (m, 2H, CH₂), 0.92 (m, 6H, 2CH₃); ¹³C NMR (D₂O): 164.3 (CO—O), 163.6 (CO—N), 149.9 (C-6), 148.2 (C-2), 146.5 (C-4), 131.0 (C-3), 127.8 (C-5), 62.0 (CH₂—CO), 47.3 (CH), 46.6 (CH), 30.9 (CH₂), 30.1 (CH₂), 20.5 (CH₂), 20.4 (CH₂), 14.7 (CH₃), 14.6 (CH₃); MS: m/z (FAB⁺) 293 (M⁺+1).

### 3-Carboxy-1-[(3-methoxy-benzylcarbamoyl)-methyl]-pyridinium (22)

Mp: 194-196. IR(KBr): 3217, 1722, 1675, 1588, 1556, 1397, 1259, 1129, 713.3; ¹HNMR (D₂O): 9.33 (s, 1H, H-2), 9.07 (d, J=7.8, 1H, H-6), 8.96 (d, J=6.2, 1H, H-4), 8.24 (m, 1H, H-5), 7.37 (m, 1H, ArH-2), 6.98 (m, 3H, ArH-4, 5, 6), 5.62 (s, 2H, CH₂—CO), 4.47 (s, 2H, CH₂—N), 3.84 (s, 3H, CH₃); ¹³C NMR (D₂O): 164.2 (CO—O), 163.1 (CO—N), 158.2 (ArC3), 148.9 (C6), 147.4 (C2), 145.9 (C4), 139.0 (C3), 130.4 (ArC1), 129.4 (ArC5), 127.7 (C5), 119.5 (ArC6), 113.2 (ArC4), 112.3 (ArC2); MS: m/z (FAB⁺) 301.0 (M⁺+1).

### 3-Carboxy-1-dipropylcarbamoylmethyl-pyridinium (23)

Yellow oil was given after crystallization from methanol and ether.

Mp: Oil. IR: 3392, 2964, 2875, 1729, 1649, 1458, 1430, 1385, 1243, 1207; ¹H NMR (D₂O): 9.23 (s, 1H, H-6), 9.04 (d, J=8.1, 1H, H-2), 8.87 (d, J=5.1, 1H, H-4), 8.20 (m, 1H, H-5), 5.80 (s, 2H, CH₂—CO), 3.34 (m, 4H, 2CH₂), 1.71 (m, 2H, CH₂), 1.54 (m, 2H, CH₂), 0.92 (t, J=7.4, CH₃), 0.82 (t, J=7.4, CH₃). ¹³C NMR (D₂O): 164.7 (CO—O), 164.6 (CO—N), 148.6 (C6), 147.4 (C2), 146.8 (C4), 132.1 (C3), 128.1 (C5), 62.1 (CH₂—CO), 49.4 (CH₂), 48.9 (CH₂), 21.4 (CH₂), 20.4 (CH₂), 10.9 (CH₃), 10.7 (CH₃); MS: m/z (FAB⁺) 266.3 (M⁺+1).

### 3-Carboxy-1-decylcarbamoylmethyl-pyridinium (24)

Mp: 185-186. IR: 3229, 3079, 2924, 1726, 1679, 1554, 1394, 1225, 1130, 846, 723; ¹H NMR (DMSO): 9.53 (s, 1H, H-2), 9.19 (d, J=5.4, 1H, H-6), 9.03 (d, J=8.1, 1H, H-4), 8.70 (s, brs, 1H, NIH), 8.31 (m, 1H, H-5), 5.57 (s, 2H, CH₂—CO), 3.13 (m, 2H, CH₂—N), 1.24-1.61 (m, 16H, 8CH₂), 0.83 (m, 3H, CH₃); ¹³C NMR (DMSO): 164.5 (CO—O), 163.6 (CO—N), 149.7 (C-6), 147.9 (C-2), 146.4 (C-4), 131.1 (C-3), 128.8 (C-5), 62.4 (CH₂—CO), 32.2 (CH₂—N), 29.9 (CH₂), 29.8 (CH₂), 29.7 (CH₂), 29.6 (2CH₂), 27.2 (CH₂), 23.0 (CH₂), 14.9 (CH₃); MS m/z

### Example 14

### 1-Allyl-3-carboxy-pyridinium bromide

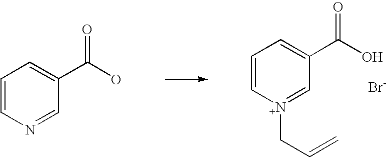

Nicotinic acid (100 mg, 0.812 mmol) and allyl bromide (70 μL, 0.812 mmol) were reacted under the general protocol to afford a yellow solid which was recrystallized from methanol to afford the desired quarternised product as a white solid (165 mg, 83%) which showed δ_{H} (400 MHz, D₂O) 9.20 (1H, s, H2), 8.81 (2H, d, J 5.8, 2×CH), 8.0 (1H, d, J 8.2, H5), 5.91-6.01 (1H, m, :CH), 5.37 (2H, app t, J 10.5, :CH₂) and 5.11 (2H, d, J 6.2, CH₂—N). δ_{C} (100 MHz, D₂O) 161.0 (C═O), 142.7, 141.9, 141.6 (all CH), 128.8 (C—CO₂H), 125.5, 124.2 (both CH), 119.4 (:CH₂) and 59.7 (CH₂). m/Z [ES] 164.0 (M⁺, 55%).

### Example 15

### 1-Propyl-3-carboxy-pyridinium iodide

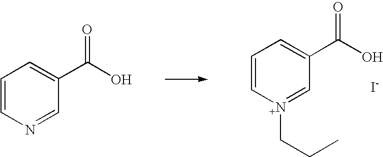

Nicotinic acid (100 mg, 0.812 mmol) and 2-iodopropane (80 μL, 0.812 mmol) were reacted under the general protocol to afford a yellow solid which was recrystallized from methanol to afford the desired quarternised product as a white solid (181 mg, 76%) m.p. 162-165° C. which showed δ_{H} (400 MHz, D₂O) 9.30 (1H, s, H2), 8.91-8.96 (2H, m, 2×CH), 8.10-8.17 (1H, m, H5), 4.61 (2H, t, J 7.4, CH₂—N), 2.03 (2H, sextet, J 7.4, CH₂) and 0.92 (3H, t, J 7.4, CH₃). δ_{C} (100 MHz, D₂O) 165.1 (C═O), 146.2, 144.8, 142.9 (all CH), 133.1 (C—CO₂H), 127.7 (CH), 63.0 (CH₂—N), 23.5 (CH₂) and 8.9 (CH₃). νₘₐₓ/cm⁻¹ 3396 (CO₂H) and 1636 (C═O). m/z [ES] 167 (M⁺, 80%).

### Example 16

### 1-Benzyl-3-carboxy-pyridinium bromide

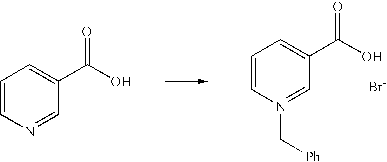

Nicotinic acid (100 mg, 0.812 mmol) and benzyl bromide (0.1 mL, 0.812 mmol) were reacted under the general protocol to afford a yellow solid which was recrystallized from water to afford the desired quarternised product as a white solid (219 mg, 92%) m.p. 203-205° C. which showed δ_{H} (400 MHz, D₂O) 9.30 (1H, s, H2), 8.80 (1H, d, J 5.9, H4), 8.75 (1H, d, J 8.2, H6), 8.01 (1H, m, H5), 7.43 (5H, m, ArH) and 5.82 (2H, s, CH₂). δ_{C} (100 MHz, D₂O) 166.1 (C═O), 146.3, 145.9, 145.5 (all CH), 132.6 (C—CO₂H), 130.2, 129.8, 129.3, 128.5 (all CH) and 65.0 (CH₂). νₘₐₓ/cm⁻¹ 3416 (CO₂H) and 1668 (C═O). m/z [FAB⁺] 214.2 (M⁺+H, 100%).

### Example 17

### 1-Carbamoylmethyl-3-carboxy-pyridinium iodide

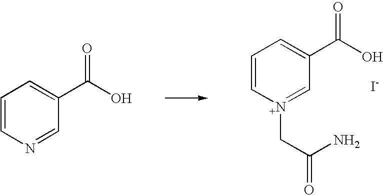

Nicotinic acid (100 mg, 0.812 mmol) and 2-iodoacetamide (150 mg, 0.812 mmol) were reacted under the general protocol to afford a yellow oil which was recrystallized from dichloromethane to afford the desired quarternised product as a yellow solid (175 mg, 70%) m.p 223-225° C. which showed δ_{H} (400 MHz, D₂O) 9.24 (1H, s, H2), 9.0 (1H, d, J 8.2, H6), 8.89 (1H, d, J 4.8, H4), 8.14-8.20 (1H, m, H5) and 5.56 (2H, s, CH₂). δ_{C} (100 MHz, D₂O) 169.5, 167.3 (both C═O), 149.3, 148.6, 148.1 (all CH), 135.6 (C—CO₂H), 129.5 (CH) and 63.2 (CH₂). νₘₐₓ/cm⁻¹ 3379 (CO₂H), 1701 (CO—NH₂) and 1665 (C═O). m/z [FAB⁺] 181.2 (M⁺+H, 100%).

### Example 18

### 3-Carbamoyl-1-carbamoylmethyl-pyridinium; iodide

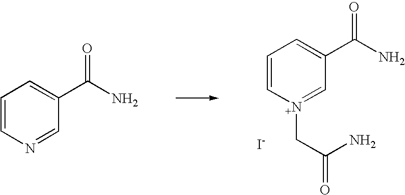

Nicotinamide (500 mg, 4.06 mmol) and iodoacetamide (764 mg, 4.06 mmol) were stirred in DMF at 50° C. overnight. The solvent was then evaporated and the residue crystallized from methanol/ether to afford a yellow solid which showed δ_{H} (270 MHz, D₂O) 9.24 (1H, s, H2), 8.95-8.93 (2H, m, H4 and H6), 8.23-8.17 (1H, m, H5) and 5.56 (2H, s, py-CH₂). δ_{C} (65 MHz, D₂O) 167.3 (C═O), 148.2, 146.1, 145.2 (all CH), 133.9 (C), 128.3 (CH) and 62.0 (CH₂). m/z [FAB⁺] 180.0 (M⁺, 100%) [found 180.0780 M+. C₆H₁₀N₂O₃I requires 180.0781].

### Example 19

### 5-Bromo-1-carbamoylmethyl-3-carboxy-pyridinium iodide

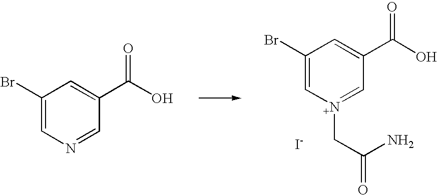

5-Bromonicotinic acid (50 mg, 0.247 mmol) and iodoacetamide (45 mg, 0.247 mmol) were stirred in DMF (1 mL) in the dark at 50° C. overnight. The solvent was evaporated under reduced pressure and the residue was crystallized from MeOH/Et₂O to afford the desired product as a brown gum (48 mg, 51%) which showed δ_{H} (270 MHz, D₂O) 9.66 (1H, s, H2), 9.46 (1H, m, H6), 9.15 (1H, m, H4) and 5.58 (2H, s, CH₂). m/z [FAB⁺] 260.0 (M⁺, 60%) [found 260.0634 M⁺. C₈H₈⁷⁹BrN₂O₃ requires 260.0648].

### Example 20

### 1-Carbamoylmethyl-3-[1-(2-nitrophenyl)-ethoxycarbonyl]-pyridinium iodide

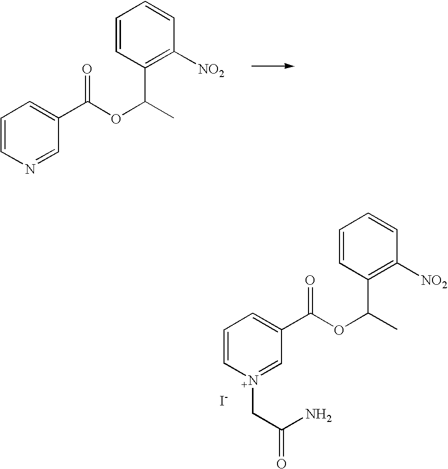

Nicotinic acid 1-(2-nitro-phenyl)-ether ester (300 mg, 1.1 mmol) and iodoacetamide (204 mg, 1.1 mmol) were stirred in the dark at 50° C. overnight. The solvent was then evaporated under reduced pressure and the residue recrystallized from MeOH/ether to afford the desired product as a yellow solid which showed δ_{H} (270 MHz, D₂O) 9.46 (1H, s, H2), 9.14 (1H, d, J 8.1, CH), 9.06 (1H, d, J 5.9, CH), 8.31-8.26 (1H, m, CH), 8.0 (1H, d, J 8.1, CH), 7.85 (1H, d, J 8.1, CH), 7.76-7.70 (1H, m, H4), 7.57-7.52 (1H, m, CH), 6.56 (1H, q, J 6.3, CH-Me) 5.64 (2H, s, CH₂) and 1.82 (3H, t, J 6.3, CH₃). δ_{C} (65 MHz, CDCl₃) 161.8 (C═O), 149.2, 147.3, 146.8 (all CH), 137.3 (C), 135.7 (C), 134.5, 129.5, 128.3, 127.6, 124.7 (all CH), 72.1, 61.9 and 20.9 (CH₃). m/z [FAB⁺] 273.1 (M⁺+H, 100%) [found 273.0871 M⁺+H. C₁₄H₁₃N₂O₄ requires 273.0873].

[E] Synthesis of Pyridinium Salts by Zincke Reaction

### 3-Carbamoyl-1-(2,4-dinitro-phenyl)-pyridinium chloride (Zincke salt)

Nicotinamide (0.5 g, 4.0 mmol) was mixed with 2,4-dinitrochlorobenzene (2.5 g, 12 mmol). This mixture was then heated at 90° C. for 2 h. After cooling down, the residual was dissolved in methanol (3 ml) and then was added ether (40 ml). The solid that followed was filtered and redissolved again in methanol (3 ml) and then ether (40 ml), this procedure was repeated four times. The solid was filtered and dried in vacuum. Orange colour foam was given in 76% yield.

IR: 1690, 1616, 1543, 1347, 1202, 704; ¹H NMR (270 MHZ, CD₃OD): 9.74 (s, 1H, H2), 9.39 (dd, J 6.2 and 1.3, 1H, H4), 9.25 (dd, J 8.4 and 1.3, 1H, H6), 9.22 (d, J 2.6, 1H, ArH), 8.86 (dd, J 8.8 and 2.6, 1H, ArH), 8.42 (dd, J 8.4 and 6.2, 1H, H5) and 8.30 (d, J 8.8, 1H, ArH). ¹³C NMR (100.4 MHz, D₂O): 165.0 (CO), 149 (C-2′), 147 (C-4′), 147.5 (C-4′), 145.7 (C-6′), 142.9 (C-2′), 138 (C-3′), 134.2 (C-1′), 131.4 (C-5), 131.0 (C-6′), 128 (C-5′), 122.9 (C-3′); MS: m/z (FAB⁺) 289 (M⁺+1), 273 (M⁺−NH₂).

Procedure for Zincke Reaction

Amines (118 mg, 0.8 mmol) were dissolved in dry methanol (20 ml). To this solution, Zincke salt (26) (270 mg, 0.83 mmol) was added. The following dark red solution was stirred at room temperature for 5 h (color from dark red to yellow). Crude products were given after removal of the solvent which were then purified by flash chromatography or crystallization.

### 1-Butyl-3-carbamoyl-pyridinium

Crude product was crystallized from methanol/ether. Yellow solid was given in 75% yield.

Mp: 205-207° C. (crystallized from methanol/ether); IR: 1669, 1647.8, 1457, 1403, 1204; ¹H NMR (270 MHz, D₂O): 9.30 (s, 1H, H-2), 9.00 (d, J=8.1, 1H, H-6), 8.87 (d, J=8.2, 1H, H-4), 8.17 (m, 1H, H-5), 4.66 (m, 2H, CH₂), 2.01 (m, 2H, CH₂), 1.38 (m, 2H, CH₂), 0.93 (m, 3H, CH₃); MS: m/z (FAB⁺) 179.0 (M⁺+1). M/z calcd for C₁₀H₁₅N₂O 179.1184 found 179.1178.

### 3-Carbamoyl-1-cyclohexyl-pyridinium

Crude product was crystallized from methanol and ether. Yellow solid was given in 67% yield.

Mp: 275-276° C. (crystallized from methanol/ether); IR: 3278, 3137, 2857, 1695, 1644, 1590, 1512, 1453, 1407, 1142, 679; ¹H NMR (270 MHz, D₂O): 9.12 (s, 1H, H-2), 8.95 (d, J=8.1, 1H, H-6), 8.77 (d, J=8.2, 1H, H-4), 8.01 (m, 1H, H-5), 1.13-2.05 (m, 11H, ring-H); ¹³C NMR (100.4 MHz, D₂O): 163.3 (CO), 145.6 (C-4), 144.6 (C-6), 143.9 (C-2), 134.5 (C-3), 128.6 (C-5), 72.0 (C-1′), 33.1 (C-2′ and C-6′), 25.8 (C-3′ and C-5′), 24.9 (C-4′); MS: m/z (FAB⁺) 205.0 (M⁺+1). M/z calcd for C₁₂H₁₇N₂O 205.1341 found 205.1339.

### 3-Carboxy-1-phenyl-pyridinium

The crude product was purified by Flash chromatography, eluted with DCM:methanol 5:2. Yellow solid was given in 83% yield. Analytical data was obtained by crystallization from methanol and ether.

Mp: 235-237° C. (crystallized from methanol/ether), IR: 3370, 3140, 1687, 1491, 1398, 1256, 766, 680; ¹H NMR (270 MHz, D₂O): 9.55 (s, 1H, H-2), 9.27 (d, J=5.5, 1H, H-6), 9.06 (d, J=8.1, 1H, H-4), 8.34 (m, 1H, H-5), 7.75 (s, 5H, ArH); ¹³C NMR (100.4 MHz, D₂O): 165.7 (CO), 146.7 (C-4), 144.9 (C-6), 144.5 (C-2), 134.0 (ArC1), 132.0 (C-5), 130.6 (ArC2 and C6), 128.5 (ArC4), 124.2 (ArC3 and ArC5); MS: m/z (FAB⁺) 199.1 (M⁺+1). C₁₂H₁₁N₂O 199.0871 found 199.0871.

### 3-Carbamoyl-1-decyl-pyridinium

The cruded product was crystallized from methanol and ether. Yellow oil was given which was further purified by Silica column (DCM:methanol 5:1). Yield 66%.

IR: 3257, 3075, 2920, 2852, 1703, 1512, 1451, 1407, 1335, 1144, 805, 679; ¹H NMR (270 MHz, D₂O): 9.29 (s, 1H, H-2), 9.00 (d, J=6.0, 1H, H6), 8.88 (d, J=8.3, 1H, H4), 8.17 (dd, J=8.3, 6.0, 1H, H5), 4.78 (m, 2H, CH₂—N), 3.33 (MeOH), 2.05 (m, 2H, CH₂), 1.23 (m, 14H, 7CH₂), 0.83 (m, 3H, CH₃); ¹³C NMR (100.4 MHz, D₂O): 165.3 (CO), 146.6 (C4), 144.3 (C2), 143.9 (C6), 134.0 (C3), 128.6 (C5), 62.7 (CH₂—CO), 31.7 (CH₂), 31.2 (CH2), 29.3 (CH₂), 29.2 (CH₂), 29.1 (CH₂), 28.8 (CH₂), 25.8 (CH₂), 22.6 (CH₂), 13.9 (CH₃); MS: m/Z (FAB⁺) 263.0 (M⁺+1). M/z calcd for C₁₆H₂₇N₂O 263.2123 found 263.2126.

### 3-Carbamoyl-1-(2-hydroxy-1-hydroxymethyl-2-phenyl-ethyl)-pyridinium

Yellow oil was obtained after flash chromatography (DCM:methanol 5:1) in 100% yield.

IR: 3350, 3158, 1702, 1646, 1448, 1401, 1289, 1019, 678; ¹H NMR (270 MHz, D₂O): 9.27 (s, 1H, H2), 9.04 (d, J=6.2, 1H, H-6), 8.94 (d, J=8.4, 1H, H-4), 8.18 (m, 1H, H-5), 7.35 (m, 5H, ArH), 5.45 (m, 1H, CH—N), 5.12 (m, 1H, CH—OH), 4.14 (m, 2H, CH₂—OH); ¹³C NMR (100.4 MHz, D₂O): 165.7 (CO), 146.4 (C4), 144.9 (C2), 144.1 (C6), 138.3 (ArC1), 133.7 (C3), 129.2 (ArC3 and C5), 128.2 (C5), 126.1 (ArC2 and C6), 126.7 (ArC4); MS: m/z (FAB⁺) 273.1 (M⁺+1). HPLC: 10.20 (RP-18, WL 254 Acetonitrile/water gradient 5-5%). M/z calcd for C₁₅H₁₇N₂O₃ 273.1239 found 273.1238.

Procedure for 3-Carboxamide Hydrolysis

3-Carbamoyl-1-(2-hydroxy-1-hydroxymethyl-2-phenyl-ethyl)-pyridinium (200 mg, 1.02 mmol) was added concentrated HCl (3 ml). This reaction mixture was then heated to reflex for 2 h and then kept at r.t for overnight. Removal of solvent gave crude product, which was crystallized from acetone to give yellow solid in over 80% yield.

### 3-Carboxy-1-(2-hydroxy-1-hydroxymethyl-2-phenyl-ethyl)-pyridinium

IR: 1731, 1636, 1402, 1298, 1173, ¹H NMR (270 MHz, D₂O): 9.27 (s, 1H, H-2), 9.02 (m, 2H, H-6 and 4), 8.16 (m, 1H, H-5), 7.54-7.30 (m, 5H, ArH), 5.48 (m, 1H, CH—N), 5.12 (m, 1H, CH—OH), 4.12 (m, 2H, CH₂—OH); ¹³C NMR (100.4 MHz, D₂O): 165.6 (CO), 146.6 (C4), 146.5 (C2), 145.3 (C6), 138.3 (ArC1), 129.2 (ArC3 and C5), 128.1 (C5), 126.1 (ArC2 and C6) 79.2 (CH—N), 69.8 (CH₂—OH), 60.3 (CH—OH); MS: m/z (FAB⁺) 274 (M⁺+1).m/z calcd for C₁₅H₁₆NO₄ 274.1074 found 274.1077. HPLC: 7.45 min (RP-18, WL 254 Acetonitrile/water gradient 5-50%).

### 3-Carboxy-1-cyclohexyl-pyridinium

Yellow solid was given after crystallized from acetone. Yield 80%.

Mp: 235-237, ¹H NMR (270 MHz, D₂O): 9.56 (s, 1H, H-2), 9.49 (d, J=5.5, 1H, H-6), 8.94 (d, J=8.1, 1H, H-4), 8.28 (m, 1H, H-5), 4.86 (m, 1H, CH-1′), 2.14-1.07 (m, 11H, ring-H); ¹³C NMR (100.4 MHz, D₂O): 163.6 (CO), 146.5 (C-4), 145.9 (C-6), 145.6 (C-2), 131.8 (C-3), 129.1 (C-5), 71.9 (C-1′), 33.1 (C-2′ and C-6′), 25.8 (C-3′ and C-5′), 24.8 (C-4′); MS: m/z (FAB⁺) 206.1 (M⁺+1).

### Example 21

### Synthesis of Pyridinium Salt in Solvent System

model compound (III).

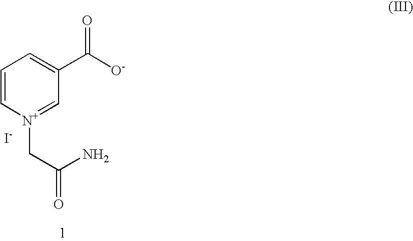

The synthesis of pyridinium salts was shown in (a). 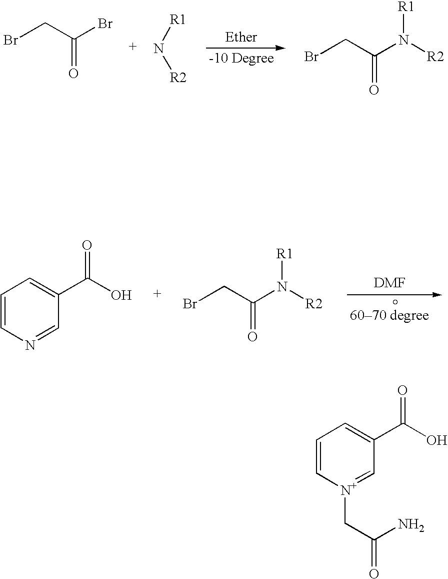
(a) Synthesis of Pyridinium Salts in Solvent System.

The Bromoacetylamides were prepared [1] in ether at −10° C. in a yield at 60-80% and the following alkylation reaction was carried out in DMF at 60-70° C. in the dark. 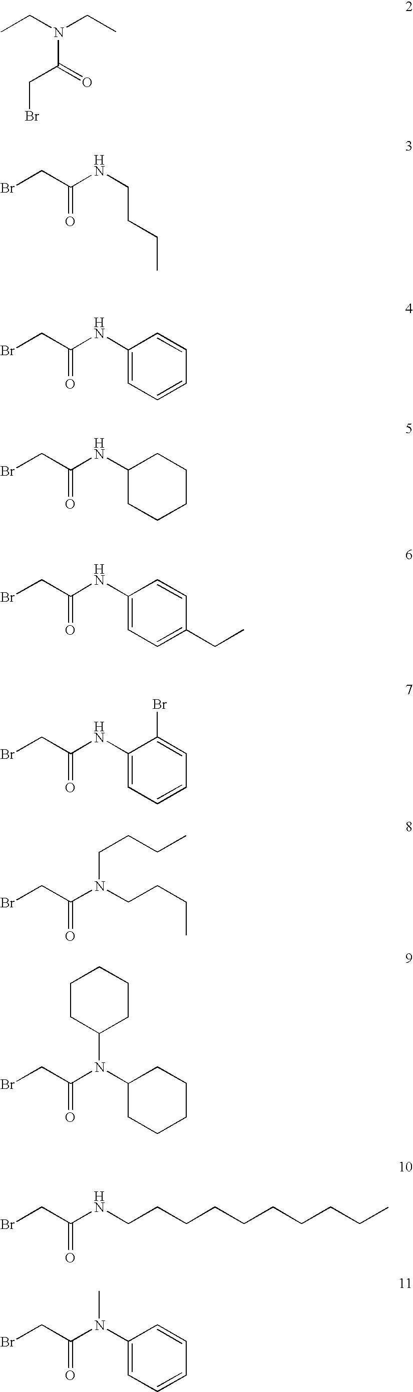 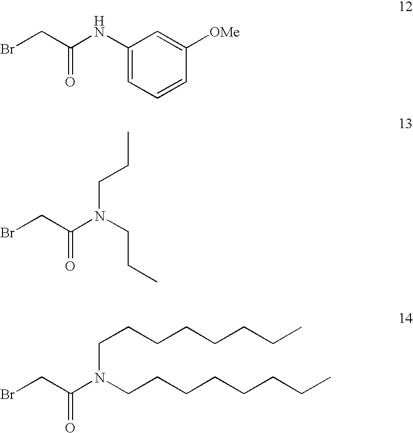
(b) List of Amides 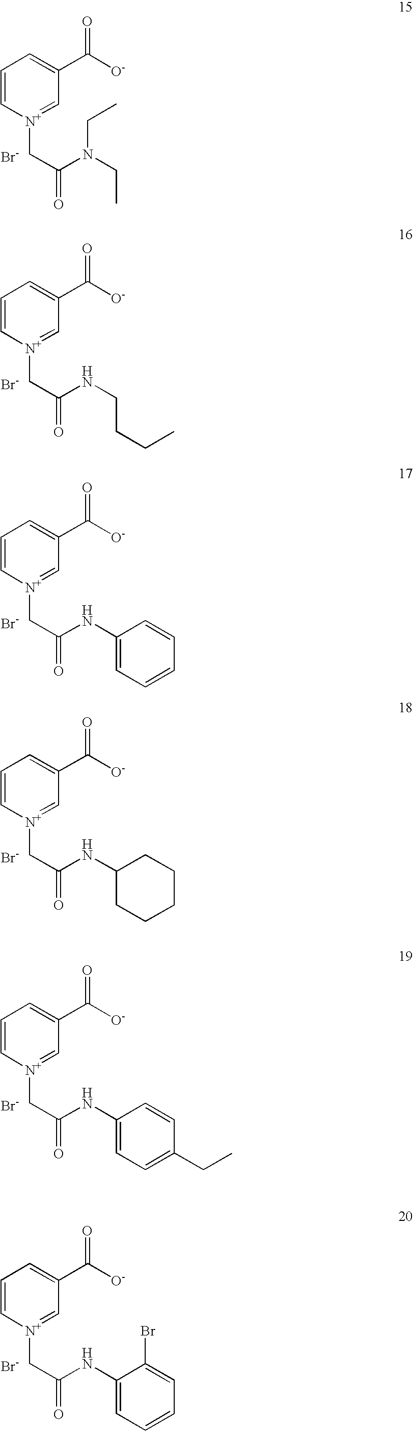 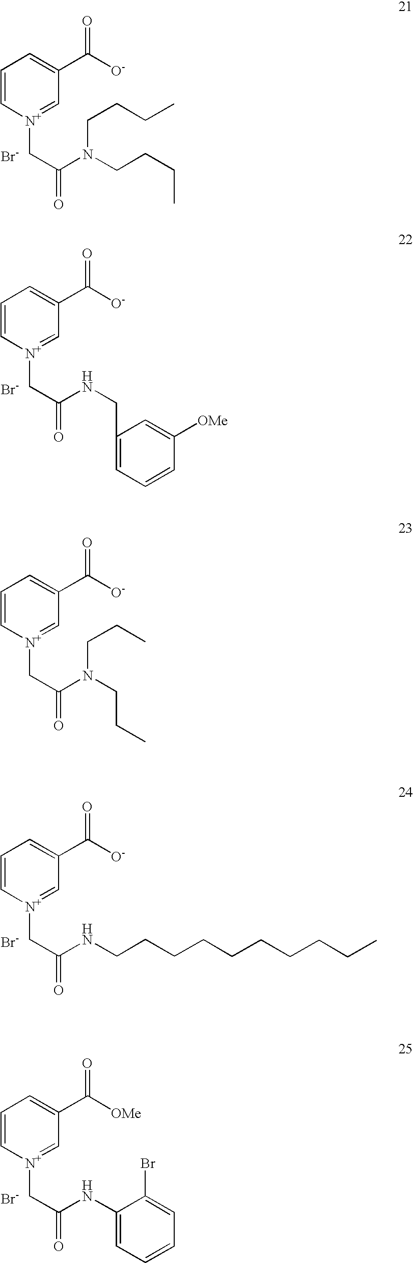
(b) List of Pyridinium Salts.

### Example 22

### HPLC of the Pyridinium Salts

The purity of the pyridinium salts was determined by HPLC [2] (RP-18; WL 254, Flow rate: 1 ml/min; acetonitrile/water 5-50% gradient). Some of the results are shown in FIG. 5.

### Example 23

### Biological Data

Biological data is presented in FIGS. 1 to 4. These experiments were carried out as described above in the description of the figures.

### Example 24

### The Effect of Externally Applied CMA008 on Calcium Release in Response to Photolysis of NPE-NAADP in Intact Sea Urchin Eggs

Sea urchin eggs of Lytechinus pictus were obtained by intracoelomic injection of 0.5 M KCl shed into artificial sea water (in mM, NaCl 435, MgCl₂ 40, MgSO4 15, CaCl₂ 11, KCl 10, NaHCO₃ 2.5, EDTA 1), dejellied by passing through 90-mm nylon mesh, and then washed twice by centrifugation. Eggs were transferred to polylysine-coated glass coverslips for microinjection and microscopy. Oregon Green 488 BAPTA (1,2-bis(2-aminophenoxy)ethane-N,N,N9,N9-tetraacetic acid dextran; Molecular Probes) was pressure-microinjected (Picospritzer; World Precision Instruments). The calcium-sensitive dye was imaged by laser-scanning confocal microscopy (Leica model TCS NT) using the 488-nm line of an argon ion laser for excitation, and the emission was long passfiltered (515 nm) and detected with a photomultiplier tube. Caged NAADP (²⁹P-(1-(2-nitrophenyl)ethyl) NAADP; Molecular Probes) was purified further by high performance liquid chromatography to remove small amounts of contaminating free NAADP. Caged NAADP were photolyzed with ultraviolet light (351- and 364-nm lines) from an argon ion laser (Enterprise model 651; Coherent) that was directed into the scanning head by a quartz fiber optic cable. The spatial location of photolysis was controlled via a shutter that was placed in the light path of the ultraviolet laser. This resulted in a band of UV across the image with the position and width of the band being controllable. The confocal images were processed with the software NIH Image to create a self ratio by dividing the intensity (F) of each image on a pixel by pixel basis by the intensity of an image acquired before stimulation (Fo). Time courses of F/Fo are plotted against time. Results are shown of the effect of externally applied CMA008 on the effect of photolysing NPE-NAADP. CMA008 (10 mM) blocks the effect of photolysis of NPE-NAADP (1 μM) on calcium release in intact sea urchin eggs, supporting the notion of membrane permeance of CMA008. The biological data for these experiments is presented in FIG. 6 (see also the description of the figures above).

### Example 25

### Effect of Externally Applied CMA008 (1-Carbamoylmethyl-3-carboxy-pyridinium iodide) on CCK-Induced Oscillations of Ca²⁺ in Pancreatic Acinar Cells from Single Cell Imaging Measurements

Pancreatic acinar cells were seeded onto poly-lysine-coated number 1 glass coverslips and loaded with calcium indicator by incubating cells with 1-5 mM fura-2 acetoxymethylester (Molecular Probes; Leiden, Holland) for 60 min at room temperature. After the loading period, cells were subsequently washed and maintained in buffer at room temperature and used immediately. Cells were excited alternately with 340 and 380 nm light (emission 510 nm), and ratio image of clusters 5 were recorded every 4-5 s, using a 12-bit CCD camera (MicroMax; Princeton Instruments, NJ).

All experiments were conducted at room temperature. CMA008 (1-Carbamoylmethyl-3-carboxy-pyridinium iodide) was dissolved in 50% DMSO. Final concentration of DMSO was 0.5% in the solution (composition in mM: 140 NaCl, 4.7 KCl, 1 CaCl₂, 1.13 MgCl₂, 10 HEPES, 10 Glucose, pH adjusted to 7.2). The inhibition of CCK-evoked calcium spiking, both pre- and post-applied with respect to CCK (5 pM), was consistent with inhibition of NAADP-induced calcium release by CMA008 (1 mM). The biological data for these experiments is presented in FIG. 7 (see also the description of the figures above).

### SUMMARY

Novel chemical entities that modulate the release of intracellular calcium by a novel mechanism from a specific store controlled by nicotinic acid adenine dinucleotide phosphate are described. These small molecules are cell permeable and have been shown to inter alia modulate calcium spikes in mammalian beta cells. NAADP mediated calcium stores are found in a wide range of mammalian cells including brain, heart, pancreatic acinar and T-cells. These and related compounds may thus find application as novel therapeutic agents and as probes for biological assays.

Nicotinic acid adenine dinucleotide phosphate (NAADP) displays a carboxylate at the 3-position of the pyridinium, unlike the carboxamide displayed by the related biological co-substrate nicotinamide adenine dinucleotide phosphate (NADP). It has recently emerged that low concentrations of NAADP (approx. 100 nM) causes release of calcium from a discrete intracellular store that is not addressed by any other second chemical messenger, such as cADPR or 1,4,5-InsP₃. Despite being a close analogue NADP is not active. Studies on closely related NAADP analogues confirmed strong specificity at the nicotinic acid position. Strong specificity is often accompanied by sub-sites that are responsible for tight binding interactions with the ligand and the inventors reasoned that small molecule analogues might bind at such a sub-site and by competing with the natural ligand, potentially act as modulators of this novel biological mechanism.

Simple pyridinium salts of nicotinic acid have been prepared and have been shown to modulate calcium release in model systems, such as sea urchin homogenate, as well as mammalian pancreatic cells. The compounds were applied outside of the cell, yet demonstrated potent activity so confirming that they are cell permeable.

It is believed that these novel molecules act by binding to the NAADP receptor at a sub-site responsible for binding the nicotinic acid portion of the ligand.

Such compounds provide a powerful basis for the development of novel therapeutics that act by modulating calcium signals critical to controlling a number of important biological processes, such as fertility, insulin production, T-cell activation, controlling the frequency of heart muscle contractions and the activity of brain cells.

The chemical entities described herein may be used either for assays, or themselves developed into novel pharmaceutical agents that intercept and control this important biological pathway. Examples of diseases that feature aberrant intracellular calcium signalling are manifold and include diabetes, while the role of calcium in the activation of T-cells offers the prospect of control of the immune system. That NAADP receptors have been shown to be active in the brain suggests a possible role for controlling neurological diseases.

The chemical entities described will potentially be applicable for the modulation of any disease due to aberrant NAADP induced calcium release. The general structure of the compounds may find application for other biological targets that feature related binding sites for nicotinic acid/amide derivatives.

### REFERENCES

[1] T. Hamas, D. A. Culkin, J. F. Hartwig, J. Am. Chem. Soc., 2003, 125 (37), 11176-11177.
[2] M. A. Lago, T. T. Nguyen, P. Bhatnagar, Tetrahedron Lett. 1998, 39, 3885-3888.

### Example Section Two

In this section reference is made to work done on preferred embodiments of the present invention. The same definitions as provided herein also apply to this section.

In this section reference is made to the following Figures:
FIG. 8—which presents some structures
FIG. 9—which presents some data in the form of a bar chart
FIG. 10—which presents some trace data
FIG. 11—which presents some additional trace data
FIG. 12—which presents some graphical data
FIG. 13—which presents some graphical data
FIG. 14—which presents some graphical data
FIG. 15—which presents some graphical data
FIG. 16—which presents some graphical data

In this respect, and as presented above, the invention concerns inter alia the use of compounds of the formula (I): 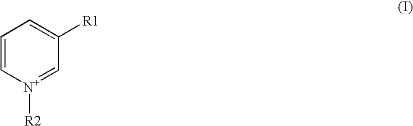
wherein:
R1 comprises a carbonyl group
R2 is a hydrocarbyl group;
   optionally wherein said ring is further substituted;
   or a pharmaceutically acceptable salt thereof;
   in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

Typically the carbonyl group is a carboxyl group.

In a particular preferred aspect the compounds are formulated and/or administered in effective amounts to patients suffering from thyroiditis, insulitis, multiple sclerosis, invectitis, orchitis, myasthenia gravis, rheumatoid arthritis, lupus erythematosis, graft rejection, Type II diabetes or cardiac arrhythmia.

In a more particular preferred aspect the compounds are formulated and/or administered in effective amounts to patients suffering from multiple sclerosis or rheumatoid arthritis or graft rejection.

In a more particular preferred aspect the compounds are formulated and/or administered in effective amounts to patients suffering from multiple sclerosis.

We have found that compounds of the formula (A1) (shown below) are particularly favourable for the treatment of thyroiditis, insulitis, multiple sclerosis, invectitis, orchitis, myasthenia gravis, rheumatoid arthritis, lupus erythematosis, graft rejection, Type II diabetes or cardiac arrhythmia; more particularly multiple sclerosis or rheumatoid arthritis; more particularly multiple sclerosis.

A compound of formula (A1): 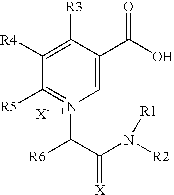
wherein X (i.e. the ═X) is O, S, CH2 or (H,H);
wherein R1 is hydrocarbyl group or an oxyhydrocarbyl group or H;
wherein R2 is hydrocarbyl group or an oxyhydrocarbyl group or H;
optionally wherein R1 and R2 can be linked so as to define a hydrocarbyl ring structure such as cyclohexyl or —(CH)n-cyclohexyl (wherein n is an integer) or heterocylic ring structures such as 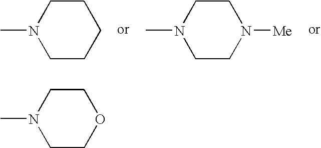
wherein R3 is H or halo or a hydrocarbyl group which may be saturated or unsaturated (such as aryl or methoxy aryl);
wherein R4 is H or halo or a hydrocarbyl group which may be saturated or unsaturated (such as aryl or methoxy aryl);
wherein R5 is H or halo or a hydrocarbyl group which may be saturated or unsaturated (such as aryl or methoxy aryl);
wherein R6 is H or halo or a hydrocarbyl group (such as an oxyhydrocarbyl group);
wherein X⁻ is an optional anion, such as Br⁻;
wherein the compound may be salt form or as a Zwitterion.

Any one or more of R3, R4, R5 and R6 can be an aryl group.

R1 need not be the same as R2. R3 need not be the same as R4 or R5 or R6. R4 need not be the same as R3 or R5 or R6. R5 need not be the same as R3 or R4 or R6. R6 need not be the same as R3 or R4 or R5.

Preferably R1 is hydrocarbyl group or an oxyhydrocarbyl group; and R2 is hydrocarbyl group or an oxyhydrocarbyl group; optionally wherein R1 and R2 can be linked so as to define a hydrocarbyl ring structure.

R3 and R4 may optionally be fused to form a ring structure.

Preferred examples of compounds falling within this scope are as follows: 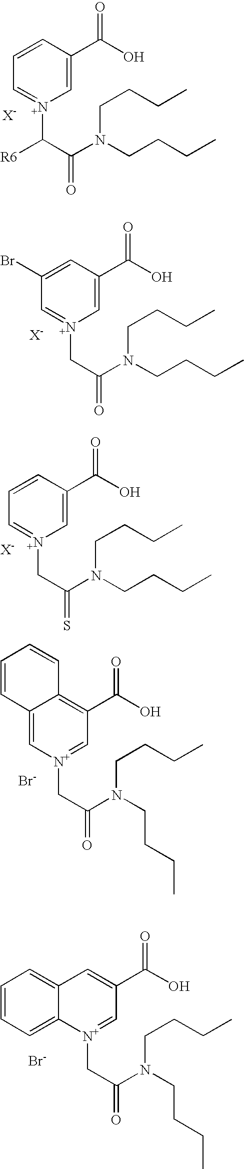
(as well as similar structures wherein the non-heterocylic ring is saturated.)

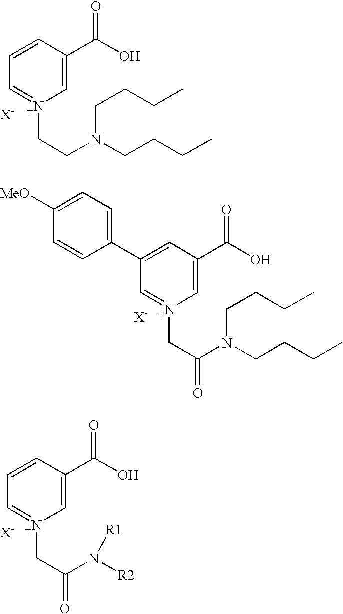

By way of further example, for the compound of the formula: 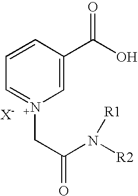
R1 can be H or C₄H₉; and R2 can be cyclohexyl. Alternatively, R1 can be H or methyl and R2 can be C₄H₉. Alternatively NR1R2 can be any of:

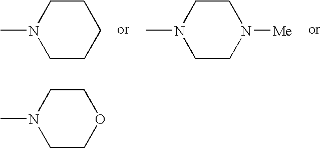

A more preferred compound of formula (A1) is that of formula (B) (as shown below) or a pharmaceutically acceptable salt thereof or a mimetic thereof or a bioisotere thereof. Sometimes the compound of formula (B) is referred to as “BZ52”. The term “mimetic” relates to any chemical which has the same qualitative activity or effect as a reference agent.

BZ52 has the formula:

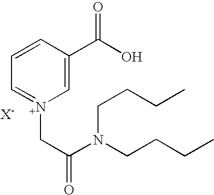

The Zwitterion form of BZ52 has the formula: 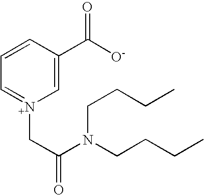
The Synthesis Details for BZ52 and Similar Structures Now Follow.
Material and Methods

Thin layer chromatography (TLC) was performed on precoated plates (Merck TLC aluminum sheets silica 60 F254) with eluants as indicated. The compounds were detected using a UV lamp at 254 nm. Flash chromatography was carried out using Sorbisil C60 silica gel. Reagents were obtained from commercial sources and used without further purification unless otherwise stated.

¹H and ¹³C NMR spectra were recorded on either a JEOL GX270 or Varian 400 MHz spectrometer. Coupling constants, J, are measured in Hertz (Hz) and the following abbreviations have been used: s, singlet; d, doublet; t, triplet; q, quartet; m, multiplet; br, broad. Unless otherwise stated chemical shifts were measured in parts per million (ppm) relative to residual protonated solvent.

Melting points were determined using a Reichert Jung Thermo Galen Kofler block and are uncorrected. Mass spectra were recorded using positive and negative fast atom bombardment (FAB) with 3-nitro benzyl alcohol (NBA) as a matrix carrier.
Synthesis of the Pyridinium Salts

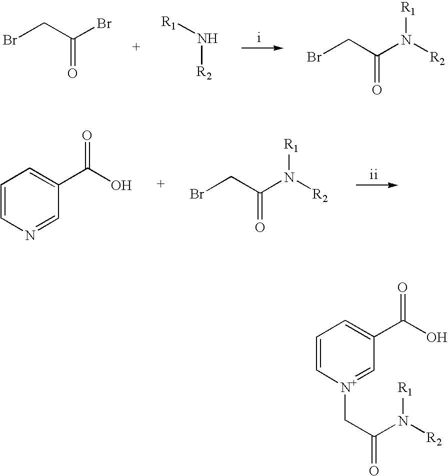

Synthesis of pyridinium salts: i) Ether, −10° C., 0.5 h; ii) DMF, 60° C., overnight in the dark.

Procedure of Preparing Bromoacetylamides.

Amine (3 ml, 24 mmol) was added dropwise to a stirring solution of 2-bromo-acetylbromide (10 ml, 12 mmol) at −10° C. The temperature of the reaction mixture was kept below 0° C. to avoid any possible side reactions. When the reaction was complete, cold water was added to dissolve the precipitated inorganic salt. The organic layer was separated and washed successively with aqueous acetic acid (20 ml, 1M), aqueous NaOH (20 ml, 1M) and finally with saturated brine. After removal of the ether, the crude product was used for alkylation directly without further purification. Analytical data was obtained after recrystallization or flash chromatography.

### Example—BZ52

### 2-Bromo-N,N-dibutylacetamide

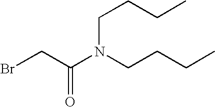

IR: 2959, 2932, 2872, 1648, 1460, 1376, 1209, 1098; ¹H NMR (270 MHz, CDCl₃): 3.77 (s, 2H, CO—CH₂), 3.23 (m, 4H, 2NCH₂), 1.48 (m, 4H, 2CH₂), 1.25 (m, 4H, 2CH₂), 0.86 (m, 6H, 2CH₃); ¹³C NMR (100.4 MHz, CDCl₃): 166.28, 48.86, 46.22, 31.60, 29.70, 26.97, 20.46, 14.27, 14.19; MS m/z (FAB⁺) 250.0 (M⁺+1), 170 (M⁺−Br), 128 (M⁺−BrCOCH₂); HRMS (FAB, positive) calcd. for (M+H)⁺ C₁₀H₂₁⁷⁹BrNO 250.08065 found 250.07976; calcd for (M+H)⁺ C₁₀H₂₁⁸¹BrNO⁺ 252.07861 found 252.07828

General Procedure for Alkylation Reaction

1 equivalent of nicotinic acid (or a derivative thereof prepared by methods known to those versed in the art) and 1 equivalent of 2-bromoacetylamide is dissolved in dry DMF (4 ml). The reaction mixture is then heated at 60° C. for 12 h in dark. After removal of the DMF by evaporation under reduced pressure, the product is obtained as an oil after flash chromatography or is crystallized from methanol/ether.

### 3-Carboxy-1-dibutylcarbamoylmethyl-pyridinium (BZ52)

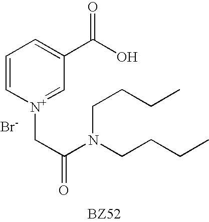

IR: 2924, 2854, 1720, 1459, 1377, 1215; ¹H NMR (270 MHz, D₂O): 9.26 (s, 1H, H-2), 9.10 (m, 1H, H-6), 8.90 (m, 1H, H-4), 8.24 (m, 1H, H-5), 5.82 (s, 2H, COCH₂), 3.44 (m, 4H, 2CH₂), 1.72 (m, 2H, CH₂), 1.54 (m, 2H, CH₂), 1.40 (m, 2H, CH₂), 1.30 (m, 2H, CH₂), 0.92 (m, 6H, 2CH₃); ¹³C NMR (100.4 MHz, D₂O): 164.25, 163.60, 149.94, 148.20, 146.54, 131.01, 128.30, 62.03, 47.26, 46.60, 30.93, 30.14, 20.51, 20.44, 14.66, 14.64; MS: m/z (FAB⁺) 293 (M⁺+1); HRMS (FAB, positive) calcd. for C₁₆H₂₅N₂O₃+293.18597 found 293.18757

Biological Test Data for BZ52 Now Follow.

Immunosuppressive Potential of BZ52-Type NAADP Antagonists

1. Background

Autoimmune diseases/chronic inflammations can be treated by immunsuppressive drugs. However, many of the drugs used today are not very specific (steroidal immunosuppressants; cytotstatic drugs) or do have severe side effects (cyclosporine A). T cell activation is a major event in the auto-immune response. We have identified a novel important signalling pathway in T cell activation, the nicotinic acid adenine dinucleotide phosphate (NAADP)/Ca²⁺ pathway.

The present invention concerns inter alia the blockade of the (NAADP)/Ca²⁺ signalling pathway by analogues of NAADP for the treatment of autoimmune diseases/chronic inflammations and graft-host rejections.

2. Inhibitory Effect of BZ 52 on Ca²⁺ Signalling in T Cells

Microinjection of NAADP into Jurkat T cells induced a rapid (peak) and sustained (plateau) Ca²⁺ signal that was not seen when intracellular buffer was injected (FIG. S2.2). Co-injection of BZ52 almost completely antagonized the effect of NAADP (FIG. S2.2). Evidence for the specificity of the inhibitory effect of BZ52 was obtained by co-injection with the Ca²⁺ mobilizing second messenger D-myo-inositol 1,4,5-trisphosphate (IP3). In these experiments IP3 induced a similar Ca²⁺ signal consisting of a rapid (peak) and sustained (plateau) phase as compared to NAADP. However, no difference between the presence and absence of BZ52 was observed (FIG. S2.2).

FIG. 9 shows the inhibitory effect of BZ 52 on Ca²⁺ signalling evoked by microinjection of Ca²⁺ mobilizing second messengers in human Jurkat T cells

NAADP (final concentration 100 nM), IP3 (final concentration 4 μM), and BZ52 (final concentration 1 mM) were injected into single Jurkat T cells while [Ca²⁺]ᵢ was analyzed by Ca²⁺ imaging. Data are presented as mean±SEM (n=4 to 13).

FIG. 10 shows the calcium traces from an individual cell microinjected with BZ52 as described for FIG. 9. Effect of NAADP-microinjection and co-injection with BZ52 (1 mM) on [Ca²⁺]ᵢ in human Jurkat T-Lymphocytes. Upper panel: microinjection of 100 nM NAADP (arrow), middle panel: microinjection of the vehicle buffer (arrow), lower panel: co-microinjection of 100 nM NAADP plus 1 mM BZ52 (arrow).

Assuming that the NAADP/Ca²⁺ signalling system plays an important role in T cell receptor/CD3 mediated activation, one would expect an inhibitory effect of BZ 52 on stimulation of rat encephalitogenic CD4-positive T cells via CD3 crosslinking. Thus, Fura2-loaded rat encephalitogenic CD4-positive T cells (resting) were stimulated by anti-CD3 followed by crosslinking of the primary antibody (FIG. 11, black line). A rapid high increase of [Ca²⁺]ᵢ followed by a briefly elevated sustained phase was observed. Preincubation of the cells for 60 min with 500 μM BZ52 significantly inhibited Ca²⁺ signalling upon CD3-crosslinking (FIG. 11, gray line)

FIG. 11 shows the inhibitory effect of BZ 52 on Ca²⁺ signalling evoked by crosslinking of CD3 in rat encephalitogenic rat T cells

Fura2-loaded rat T cells were analyzed for [Ca²⁺]ᵢ by fluorimetry. First arrow indicates addition of anti-CD3 mAb, second arrow indicates addition of a crosslinking secondary antibody. In case of BZ 52 cells were preincubated with BZ 52 (500 μM) for 60 min at RT.

3. Inhibitory Effect of BZ52 on Antigen and Mitogen Induced Proliferation in Myelin-Basic Protein (MBP) Specific, CD4-Positive Rat T Cells

A more downstream event of T cell activation was analyzed in myelin-basic protein (MBP) specific, CD4-positive rat T cells. These cells can be driven into proliferation by MBP presented by irradiated thymocytes or by the mitogen concanavalin A (ConA).

BZ52 concentration-dependently blocked both MBP- and ConA-mediated proliferation. IC₅₀ values were approx. 100 μM for ConA mediated and 500 μM for MBP mediated proliferation (FIG. 12).

FIG. 12 shows the inhibitory effect of BZ52 on antigen and mitogen induced proliferation in myelin-basic protein (MBP) specific, CD4-positive rat T cells

Data are presented as mean±SD from one representative experiment carried out in quadruplicates.

In contrast to its inhibitory effect on proliferating T cells (FIG. 12), BZ52 up to 500 μM did not much reduce the number of T cell blasts when incubated with these cells for 48 h (FIG. 13). This indicates that BZ52 does not have a large cytotoxic effect per se on resting cells, but such as affects proliferating T cells.

FIG. 13 shows the toxicity assay of BZ52 on non-proliferating myelin-basic protein (MBP) specific, CD4-positive rat T cell blasts

Data are presented as mean±SD from one representative experiment carried out in triplicates.

4. Protective Effect of BZ52 in the Multiple Sclerosis Model Experimental Autoimmune Encephalomyelitis (EAE)

Transfer of MBP-specific CD4-positive T cells was used at day 0 to induce experimental autoimmune encephalomyelitis (EAE) in Lewis rats. Animals injected with PBS (vehicle) or nicotinic acid (control compound with structural similarities to BZ52) developed severe paralysis of tail and rear legs due to the inflammation. Clinical symptoms started day 3.5 and reached their maxima day 4 to 6 (or 6.5). Between day 3.5 and day 6, no difference between PBS and nicotinic acid was observed, whereas a beneficial effect of BZ52 was visible (FIG. 14). Animals treated with BZ52 showed a less rapid increase of symptoms, a decreased maximum score and a more rapid decline of clinical symptoms as compared to the other two groups (FIG. 14).

FIG. 14 shows the protective effect of BZ52 in transfer experimental autoimmune encephalomyelitis (EAE)

Animals (6 per group, body weight approx. 150 g) were injected i.p. twice per day with either PBS (vehicle control), nicotinic acid (50 μmol/100 g body weight), or BZ 52 (50 μmol/100 g body weight). Clinical scores indicate the degree of paralysis of tail and legs. Data are presented as mean±SD from one representative experiment (n=6).

The beneficial effect of BZ52 is also mirrored by a less pronounced decrease in body weight, a marker for the disease that is less operator-based as compared to clinical scores (FIG. 15).

FIG. 15 shows the protective effect of BZ52 in transfer experimental autoimmune encephalomyelitis (EAE)

Animals (6 per group, body weight approx. 150 g) were injected i.p. twice per day with either PBS (vehicle control), nicotinic acid (50 μmol/100 g body weight), or BZ 52 (50 μmol/100 g body weight). Body weight was set to zero on day zero and changes relative to the initial body weight are displayed. Data are presented as mean±SD from one representative experiment (n=6).

Since GFP-transfected encephalitogenic T cells were used for the transfer EAE experiment, the localization of these cells was determined on day 4 post transfer in different organs of one animal. It is known from previous studies (Flügel, Immunity) that the transferred encephalitogenic T cells first move to the parathymic lymph nodes, and then migrate via the spleen to the CNS (day 4). FIG. 16 demonstrates that the number of CNS-infiltrating encephalitogenic T cells decreased by about 50%. In addition, the number of encephalitogenic T cells was reduced significantly.

FIG. 16 shows the effect of BZ52 on the localization of encephalitogenic T cells on day 4 post transfer in transfer experimental autoimmune encephalomyelitis (EAE)

Animals (6 per group, body weight approx. 150 g) were injected i.p. twice per day with either PBS (vehicle control), nicotinic acid (50 μmol/100 g body weight), or BZ 52 (50 μmol/100 g body weight). One animal per group was sacrificed on day 4 and the number of GFP-transfected cells was determined for the organs displayed. Data are corrected for organ mass and presented as mean±SD from one representative experiment.

Methods

Ratiometric Ca²⁺ Imaging of Jurkat T Cells

The Jurkat T cells were loaded with fura-2/AM and kept in the dark at room temperature until use. Glass coverslips were coated first with BSA (5 mg/ml) and subsequently with poly-L-lysine (0.1 mg/ml). Small chamber slides consisting of a rubber O-ring were sealed on thin (0.1 mm) glass coverslips by silicon grease. Then, 60 μl buffer A containing 140 mM NaCl, 5 mM KCl, 1 mM MgSO₄, 1 mM CaCl₂, 1 mM NaH₂PO₄, 5.5 mM glucose, and 20 mM HEPES (pH 7.4), and 40 μl cell suspension (2*10⁶ cells/ml) suspended in the same buffer were added into the small chamber [14] and the coverslip was mounted on the stage of a fluorescence microscope (Leica DM IRE2).

Ratiometric Ca²⁺ imaging was performed as described earlier. In brief, we used an Improvision imaging system (Heidelberg, Germany) built around the Leica microscope at 100-fold magnification. Illumination at 340 and 380 nm was carried out using a monochromator system (Polychromator IV, TILL Photonics, Graefelfing, Germany). Images were taken with a gray-scale CCD camera (type C4742-95-12ER; Hamamatsu, Enfield, United Kingdom; operated in 8-bit mode). The optimal relation of spatial to temporal resolution for the ratiometric measurements was obtained using a spatial resolution of 512×640 pixel, resulting in a pixel length of 129 nm/pixel (at 100-fold magnification). The acquisition rate was 1 ratio/sec. Raw data images were stored on hard disk. Ratio images (340/380) were constructed pixel by pixel, and finally, ratio values were converted to Ca²⁺ concentrations by external calibration. Data processing was performed using Openlab software v3.0.9, (Improvision, Tübingen, Germany).

Microiniection into Jurkat T Cells

Microinjections were carried out as described. Briefly, we used an Eppendorf system (transjector type 5246, micromanipulator type 5171, Eppendorf-Netheler-Hinz, Hamburg, Germany) with Femtotips II as pipettes. NAADP was diluted to its final concentration in intracellular buffer (20 mM HEPES, 110 mM KCl, 2 mM MgCl₂, 5 mM KH₂PO₄, 10 mM NaCl, pH 7.2) and filtered (0.2 μm) before use. Injections were made using the semiautomatic mode of the system with the following instrumental settings: injection pressure 60 hPa, compensatory pressure 30 hPa, injection time 0.3-0.5 s and velocity of the pipette 700 μm/s. Under such conditions the injection volume was 1-1.5% of the cell volume.

Ca²⁺ Measurements in Intact T Cell Suspensions

Intact Jurkat T-lymphocytes were loaded with fura2/AM. Ratiometric determination of [Ca²⁺]ᵢ was carried out in cell suspension in a Hitachi F2000 fluorometer at room temperature at excitation wavelengths of 340 and 380 nm (alternating) and an emission wavelength of 495 nm. Each experiment was calibrated by addition of Triton X100 (10% v/v final concentration) to obtain the maximal ratio and subsequent addition of 4 mM EGTA/40 mM Tris-base to obtain the minimal ratio.

In Vitro Proliferation Assay (Rat Encephalitogenic T Cells)

Resting MBP-GFP T cells were plated in 96-wells (5×10⁴ well) and stimulated by the addition of thymocytes (1.5×10⁶/well) and MBP (10 g/ml) or ConA (1.25 μg/ml), respectively.

After 2 days 3H-thymidine (Amersham Biosciences) was added to the activated cells (final concentration 4 μCi/ml); after 16 h of incubation 3H-thymidine incorporation was measured (Matrix 9600-Direct Beta Counter Packad).

In Vitro Toxicity Assay (Rat Encephalitogenic T Cells):

Resting MBP-GFP Tcells were plated in 96-wells (5×10⁴/well). Substances to test were added at different concentration to the cells and incubated for 1 h.

Cells stimulated by the addition of thymocytes (1.5×10⁶/well) and MBP.

Absolute numbers of MBP-GFP T cells were determined by quantitative cytofluorometrical analysis after 24 and 48 hours (FACS-Calibur BektonDickinson).

Activated MBP-GFP Tcells were also incubated with different concentration of substances to test and absolute numbers of MBP-GFP T cells were determined by quantitative cytofluorometrical analysis after 24 and 48 hours (FACS-Calibur BektonDickinson).

In Vivo Toxicity Assay (Lewis Rat):

Nicotinic acid or substance BZ52 were injected i.p. to recipient healthy Lewis rats twice a day for 6 days at the following concentrations: 100 μM (15 μmol substance/150 g body weight) and 500 μM (75 μmol substance/150 g body weight). Weight of the animals was measured twice a day.

Rats were sacrificed after 7 days and spleen, spinal cord and parathymic lymph nodes were prepared.

Absolute numbers of TMBP-GFP cells in the organs were determined by quantitative cytofluorometrical analysis (FACS-Calibur BektonDickinson).

EAE Induction (Lewis Rat):

Transfer of TGFP Cells:

5×10⁶ activated MBP specific-GFP Tcells were transferred i.v. into recipient Lewis rats. Clinical EAE was graded in five scores: 0.5, loss of tail tonus; 1, tail paralysis; 2, gait disturbance; 3, hind limb paralysis; 4, tetraparesis; 5, death.

Animals' weight was measured twice a day.

### REFERENCES FOR SECTION TWO

Ratiometric Ca²⁺ Imaging of Jurkat T Cells

S. Kunerth, M. F. Langhorst, N. Schwarzmann, X. Gu, L. Huang, Z. Yang, L. Zhang., S. J. Mills, L.-h. Zhang, B. V. L. Potter, A. H. Guse Amplification and propagation of pacemaker Ca²⁺ signals by cyclic ADP-ribose and the type 3 ryanodine receptor in T cells J. Cell Sci., 117, 2141-2149 (2004)
   Microinjection into Jurkat T Cells
M. F. Langhorst, N. Schwarzmann, A. H. Guse Ca²⁺ release via ryanodine receptors and Ca²⁺ entry: major mechanisms in NAADP-mediated Ca²⁺ signaling in T-lymphocytes Cell. Signal., 16, 1283-1289 (2004)
W. Dammermann, A. H. Guse. Functional ryanodine receptor expression is required for NAADP-mediated local Ca²⁺ signaling in T-lymphocytes, J. Biol. Chem. 2005 Mar. 17
   Ca²⁺ Measurements in Intact T Cell Suspensions
G. K. Wagner, S. Black, A. H. Guse, and B. V. L. Potter First enzymatic synthesis of an N1-cyclised cADPR (cyclic-ADP-ribose) analogue with an hypoxanthine partial structure: discovery of a membrane permeant cADPR agonist, Chem. Commun. 1944-1945 (2003)
N. Schwarzmann, S. Kunerth, K. Weber, G. W. Mayr, and A. H. Guse Knock-down of the type 3 ryanodine receptor impairs sustained Ca²⁺ signaling via the T cell receptor/CD3 complex J. Biol. Chem. 277, 50636-50642 (2002)
   Establishment of Encephalitogenic T Cells and tEAE Model
A. Flügel, M. Willem, T. Berkowicz, H. Wekerle. (1999) Gene transfer into CD4+ T lymphocytes: Green fluorescent protein-engineered, encephalitogenic T cells illuminate brain autoimmune responses. Nature Medicine 5, 843-847.
A. Flügel, T. Berkowicz, T. Ritter, M. Labeur, Z. Li, J. W. Ellwart, D. Jenne, M. Willem, H. Lassmann, and H. Wekerle. (2001) Migratory activity and functional changes of genetically engineered green fluorescent effector T cells before and during experimental autoimmune encephalomyelitis. Immunity 14: 547-560.

### Example Section Three—Part A

In this section reference is made to work done on preferred embodiments of the present invention. The same definitions as provided herein also apply to this section.

As indicated above, we have found that compounds of the formula (A1) (shown below) are particularly favourable for the treatment of thyroiditis, insulitis, multiple sclerosis, invectitis, orchitis, myasthenia gravis, rheumatoid arthritis, lupus erythematosis, graft rejection, Type II diabetes or cardiac arrhythmia; more particularly multiple sclerosis or rheumatoid arthritis; more particularly multiple sclerosis.

A compound of formula (A1): 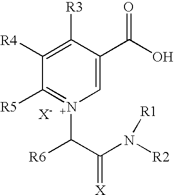
wherein X (i.e. the ═X) is O, S, CH2 or (H,H);
wherein R1 is hydrocarbyl group or an oxyhydrocarbyl group or H;
wherein R2 is hydrocarbyl group or an oxyhydrocarbyl group or H;
optionally wherein R1 and R2 can be linked so as to define a hydrocarbyl ring structure such as cyclohexyl or —(CH)n-cyclohexyl (wherein n is an integer) or heterocylic ring structures such as 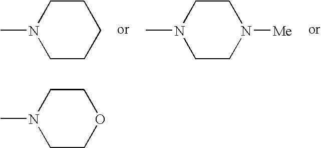
wherein R3 is H or halo or a hydrocarbyl group which may be saturated or unsaturated (such as aryl or methoxy aryl);
wherein R4 is H or halo or a hydrocarbyl group which may be saturated or unsaturated (such as aryl or methoxy aryl);
wherein R5 is H or halo or a hydrocarbyl group which may be saturated or unsaturated (such as aryl or methoxy aryl);
wherein R6 is H or halo or a hydrocarbyl group (such as an oxyhydrocarbyl group);
wherein X⁻ is an optional anion, such as Br⁻;
wherein the compound may be salt form or as a Zwitterion.

The present invention covers such compounds in per se form, in composition form, processes for making same, uses of same and methods of treatment using same as herein described.

For some aspects, a preferred compound according to the present invention has the general formula (C): 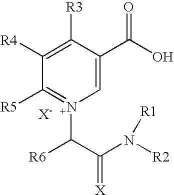
wherein X (i.e. the ═X) is 0;
wherein one of R1 and R2 is H, and the other of R1 and R2 is hydrocarbyl group;
wherein R3 is H;
wherein R4 is H;
wherein R5 is H;
wherein R6 is H;
wherein X⁻ is an anion, preferably a halo anion;
wherein the compound may be salt form or as a Zwitterion.

Examples of these compounds have already been described herein. Another example of a preferred compound of formula (C) is that of formula (D) (as shown below) or a pharmaceutically acceptable salt thereof or a mimetic thereof or a bioisotere thereof. Sometimes the compound of formula (D) is referred to as “BZ194”. The term “mimetic” relates to any chemical which has the same qualitative activity or effect as a reference agent.

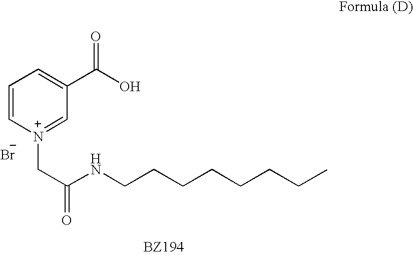

Synthesis of BZ194 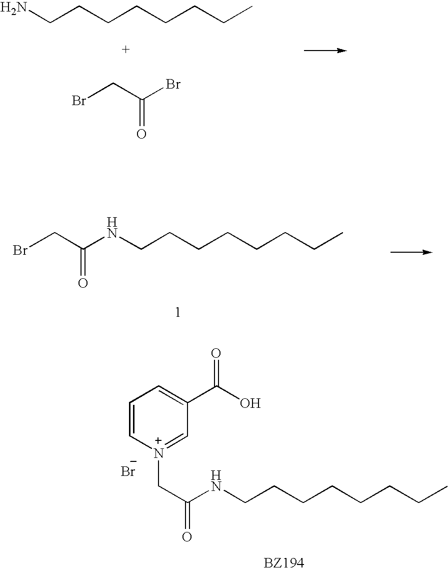
Synthetic Approach to BZ194

### 2-Bromo-N-Octylacetamide 1

A solution of bromoacetylbromide (1.0 mL, 11.5 mmol) in ether (10 mL) was cooled to −15-20° C. in an ice-salt bath, to which was added dropwise a solution of octylamine (3.8 mL, 23.0 mmol) in ether (10 mL). The resulting white suspension was stirred at this temperature for 0.5 h and then was warmed up to room temperature, followed by addition of H₂O (20 mL). The organic layer was separated and washed in sequence with solutions of HCl (2×20 mL), NaOH (2×20 mL) and H₂O (20 mL) and, was dried over MgSO₄. The solvent was removed by evaporation in vacuo and the resulting residue was further purified by flash column chromatography, eluting with a gradient of 0-10% hexane against DCM to give the title compound as a colourless oil in over 70% yield. This method was used as a general approach to synthesise similar amides with different side-chains. The yields of these amides are normally more than 60%; ¹H NMR (CDCl₃, 400 MHz) δ 6.52 (brs, 1H, NH), 3.92 (s, 2H, CH₂CO), 3.30 (s, 2H, CH₂N), 1.56 (m, 2H, CH₂), 1.31 (m, 10H, 5CH₂), 0.91 (t, J=6.5 Hz, 3H, CH₃); ¹³C NMR (CDCl₃, 100.5 MHz) δ 165.19, 40.30, 31.79, 29.47, 29.28, 29.21, 29.18, 26.83, 22.65, 14.13; HRMS Calcd. for (M+H)⁺C₁₀H₂₁⁷⁹BrNO⁺ (FAB, positive) 250.08065; found: 250.08010; Calcd. for (M+H)⁺C₁₀H₂₁⁸¹BrNO⁺ 252.0786; found: 252.0787.

### 3-Carboxy-1-Octylcarbamoylmethyl-Pyridinium (BZ194)

To a suspension of nicotinic acid (200 mg, 1.6 mmol) in anhydrous DMF (5 mL) was added 2-bromo-N-octylacetamide 1 (380 mg, 1.6 mmol). The resulting reaction mixture was heated at 60° C. and stirred in the dark overnight. The DMF was evaporated in vacuo and the residue was again dissolved in a small amount of MeOH (1-2 mL). To the resulting clear solution was added ether (20 mL) and the white precipitate was filtered and washed with ether. Purification using flash column chromatography, eluting with a gradient of 0-10% MeOH against DCM gave the title compound as a white solid in more than 80% yield. The title compound was further purified by crystallisation from MeOH/acetone giving BZ194 as needle-like crystals; ¹H NMR (DMSO₆, 270 MHz) δ 9.45 (s, 1H, H_{N}-2), 9.07 (s, J_{6,5} 5.9 Hz, 1H, H_{N}-6), 8.94 (s, J_{4,5} 8.2 Hz, 1H, H_{N}-4), 8.51 (m, 1H, NH), 8.22 (dd, J_{5,6} 5.9 Hz, J_{5,4} 8.2 Hz, 1H, H-5), 5.45 (s, 2H, CH₂CO), 3.03 (m, 2H, CH₂), 1.39-1.18 (m, 12H, 6CH₂), 0.78 (m, 3H, CH₃); ¹³C NMR (DMSO₆, 100.5 MHz) δ 164.39, 163.53, 149.66, 147.89, 146.36, 130.90, 128.12, 62.08, 31.72, 29.34, 29.17, 29.14, 26.83, 22.58, 14.45; HRMS (FAB, positive) Calcd. for C₁₆H₂₅N₂O₃+293.1860 found 293.1870; CHN: Calculated for C₁₆H₂₅N₂O₃ C, 51.48; H, 6.75; N, 7.50; found C, 51.10; H, 6.76; N, 7.33.

Testing of BZ194

BZ194 partially blocked T cell receptor/CD3 complex mediated Ca²⁺ signalling in rat and human T cells.
BZ194 had an anti-proliferative effect on cultured MBP-specific and OVA-specific T cells.
Treatment with BZ194 suppressed cytokine (mRNA) production in vitro and ex vivo.
Intraperitoneal injection of BZ194 suppressed clinical score in transfer EAE.
Infiltration of MBP-specific T cells into the CNS was reduced.
   Other Compounds of Formula (C)

Other compounds of Formula (C) may be prepared in the same manner as BZ194. For example, if either of R1 or R2 is of a different alkyl chain length, then the same synthesis protocol as described above is used except that nicotinic acid is reacted with an appropriate different alkylating reagent (i.e. a reagent that provides the required alkyl chain length).

### Example Section Three —Part B

In this section reference is made to work done on preferred embodiments of the present invention. The same definitions as provided herein also apply to this section.

As indicated above, we have found that compounds of the formula (A1) and/or formula (C) and/or formula (D) (each of which is shown below) are particularly favourable for the treatment of thyroiditis, insulitis, multiple sclerosis, invectitis, orchitis, myasthenia gravis, rheumatoid arthritis, lupus erythematosis, graft rejection, Type II diabetes or cardiac arrhythmia; more particularly multiple sclerosis or rheumatoid arthritis; more particularly multiple sclerosis.

Formula (A1): 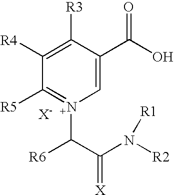
wherein X (i.e. the ═X) is O, S, CH2 or (H,H);
wherein R1 is hydrocarbyl group or an oxyhydrocarbyl group or H;
wherein R2 is hydrocarbyl group or an oxyhydrocarbyl group or H;
optionally wherein R1 and R2 can be linked so as to define a hydrocarbyl ring structure such as cyclohexyl or —(CH)n-cyclohexyl (wherein n is an integer) or heterocylic ring structures such as 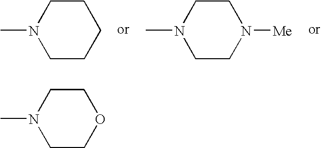
wherein R3 is H or halo or a hydrocarbyl group which may be saturated or unsaturated (such as aryl or methoxy aryl);
wherein R4 is H or halo or a hydrocarbyl group which may be saturated or unsaturated (such as aryl or methoxy aryl);
wherein R5 is H or halo or a hydrocarbyl group which may be saturated or unsaturated (such as aryl or methoxy aryl);
wherein R6 is H or halo or a hydrocarbyl group (such as an oxyhydrocarbyl group);
wherein X⁻ is an optional anion, such as Br⁻;
wherein the compound may be salt form or as a Zwitterion.

Formula (C): 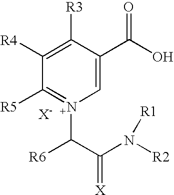
wherein X (i.e. the ═X) is 0;
wherein one of R1 and R2 is H, and the other of R1 and R2 is hydrocarbyl group;
wherein R3 is H;
wherein R4 is H;
wherein R5 is H;
wherein R6 is H;
wherein X⁻ is an anion, preferably a halo anion;
wherein the compound may be salt form or as a Zwitterion.

Formula (D):

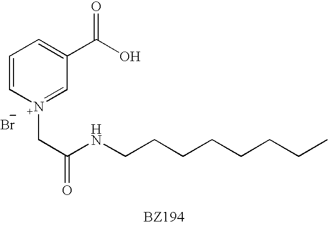

The present invention also covers such compounds in per se form, in composition form, processes for making same, uses of same and methods of treatment using same as herein described.

We have found that compounds of the formula (A2) are very favourable for the treatment of thyroiditis, insulitis, multiple sclerosis, invectitis, orchitis, myasthenia gravis, rheumatoid arthritis, lupus erythematosis, graft rejection, Type II diabetes or cardiac arrhythmia; more particularly multiple sclerosis or rheumatoid arthritis; more particularly multiple sclerosis.

Formula (A2): 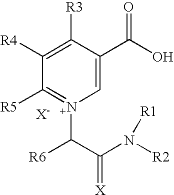
wherein X (i.e. the ═X) is O, S, CH₂, or (H, H);
wherein one of R1 and R2 is H or a hydrocarbyl group, and the other of R1 and R2 is a C5-C9 hydrocarbyl group or a C5-9 oxyhydrocarbyl group;
wherein R3 is H, halo or a hydrocarbyl group or a halo group;
wherein R4 is H, halo or a hydrocarbyl group or a halo group;
wherein R5 is H, halo or a hydrocarbyl group or a halo group;
wherein R6 is H, halo or a hydrocarbyl group or a halo group;
wherein X⁻ is an anion, preferably a halo anion, preferably Br—;
wherein the compound may be salt form or as a Zwitterion.

R1 or R2 may terminate in a cyclic group—such as:

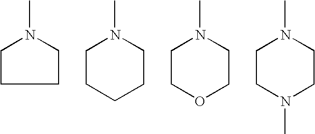

In this instance, R1 or R2 are still considered to be a C5-C9 hydrocarbyl group or a C5-9 oxyhydrocarbyl group since the terminal cyclic groups are substituents on the hydrocarbyl group or the oxyhydrocarbyl group.

R3 may be H, halo or a hydrocarbyl group—such as a saturated ring structure or an unsaturated ring structure such as aryl, methoxy aryl etc.

R4 may be H, halo or a hydrocarbyl group—such as a saturated ring structure or an unsaturated ring structure such as aryl, methoxy aryl etc.

R5 may be H, halo or a hydrocarbyl group—such as a saturated ring structure or an unsaturated ring structure such as aryl, methoxy aryl etc.

R6 may be H, halo or a hydrocarbyl group—such as a saturated ring structure or an unsaturated ring structure such as aryl, methoxy aryl etc.

For some embodiments, any two or more of R3, R4, R5 and R6 may be fused to form a saturated ring structure or an unsaturated ring structure.

Preferred compounds of formula (A2) are of the following formula (A3): 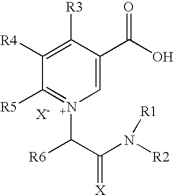
wherein X (i.e. the ═X) is O, S, CH₂, or (H, H);
wherein one of R1 and R2 is H or Me or Et, and the other of R1 and R2 is a C5-9 hydrocarbyl group or a C5-9 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.

Preferred compounds of formula (A2) are of the following formula (A4): 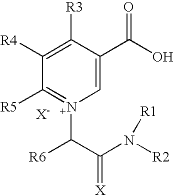
wherein X (i.e. the ═X) is O, S, CH₂, or (H, H);
wherein one of R1 and R2 is H or Me or Et, and the other of R1 and R2 is a C5-9 hydrocarbyl group or a C5-9 oxyhydrocarbyl group;
wherein R3 is H or a hydrocarbyl group;
wherein R4 is H or a hydrocarbyl group;
wherein R5 is H or a hydrocarbyl group;
wherein R6 is H or a hydrocarbyl group;
wherein X⁻ is an anion, preferably a halo anion, preferably Br—;
wherein the compound may be salt form or as a Zwitterion.

Preferred compounds of formula (A2) are of the following formula (A5): 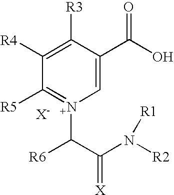
wherein X (i.e. the ═X) is O, S, CH₂, or (H, H);
wherein one of R1 and R2 is H or Me or Et, and the other of R1 and R2 is a C5-9 hydrocarbyl group or a C5-9 oxyhydrocarbyl group;
wherein R3 is H;
wherein R4 is H;
wherein R5 is H;
wherein R6 is H;
wherein X⁻ is an anion, preferably a halo anion, preferably Br—;
wherein the compound may be salt form or as a Zwitterion.

Preferred compounds of formula (A2) are of the following formula (A6): 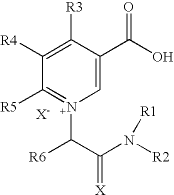
wherein X (i.e. the ═X) is O, S, CH₂, or (H, H);
wherein one of R1 and R2 is H, and the other of R1 and R2 is a C5-9 hydrocarbyl group or a C5-9 oxyhydrocarbyl group;
wherein R3 is H;
wherein R4 is H;
wherein R5 is H;
wherein R6 is H;
wherein X⁻ is an anion, preferably a halo anion, preferably Br—;
wherein the compound may be salt form or as a Zwitterion.

Preferred compounds of formula (A2) are of the following formula (A7):

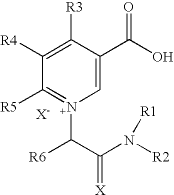

wherein X (i.e. the ═X) is O, S, CH₂, or (H, H);

wherein one of R1 and R2 is H or Me or Et, and the other of R1 and R2 is a C5 hydrocarbyl group or a C5 oxyhydrocarbyl group;

wherein R3 is H or a hydrocarbyl group or a halo group;

wherein R4 is H or a hydrocarbyl group or a halo group;

wherein R5 is H or a hydrocarbyl group or a halo group;

wherein R6 is H or a hydrocarbyl group or a halo group;

wherein X⁻ is an anion, preferably a halo anion, preferably Br—;

wherein the compound may be salt form or as a Zwitterion.

Preferred compounds of formula (A2) are of the following formula (A8): 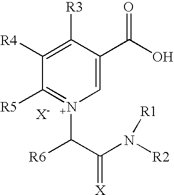
wherein X (i.e. the ═X) is O, S, CH₂, or (H, H);
wherein one of R1 and R2 is H or Me or Et, and the other of R1 and R2 is a C6 hydrocarbyl group or a C6 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.

Preferred compounds of formula (A2) are of the following formula (A9): 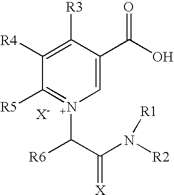
wherein X (i.e. the ═X) is O, S, CH₂, or (H, H);
wherein one of R1 and R2 is H or Me or Et, and the other of R1 and R2 is a C7 hydrocarbyl group or a C7 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.

Preferred compounds of formula (A2) are of the following formula (A10): 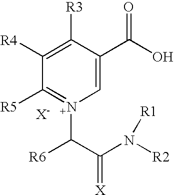
wherein X (i.e. the ═X) is O, S, CH₂, or (H, H);
wherein one of R1 and R2 is H or Me or Et, and the other of R1 and R2 is a C8 hydrocarbyl group or a C8 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.

Preferred compounds of formula (A2) are of the following formula (All): 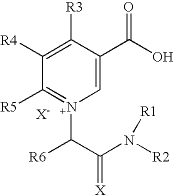
wherein X (i.e. the ═X) is O, S, CH₂, or (H, H);
wherein one of R1 and R2 is H or Me or Et, and the other of R1 and R2 is a C9 hydrocarbyl group or a C9 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.

The present invention also covers such compounds in per se form, in composition form, processes for making same, uses of same and methods of treatment using same as herein described.

For compounds of Formula (A2), preferably:

X⁻ is Br⁻ or any standard anion

X═O, S, CH₂, or (H, H)

R₁═H, Me, Et;

R₂=hydrocarbyl or oxyhydrocarbyl based around hydrocarbon chain lengths C5 to C9. The C5 to C9 hydrocarbyl or oxyhydrocarbyl groups may terminate in any one of the following groups:

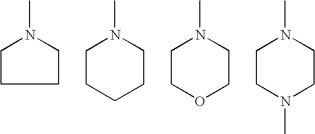

Any of R₃ or R₄ or R₅ is H or halo.

Additional examples of compounds of the present invention are presented below:

R1 ═H, Me, Et

R₂═C5-C9 hydrocarbyl

or 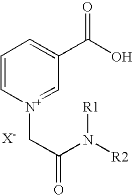
R₂=—(CH₂)ₙNR₁R₂,
Where n=5-9 and 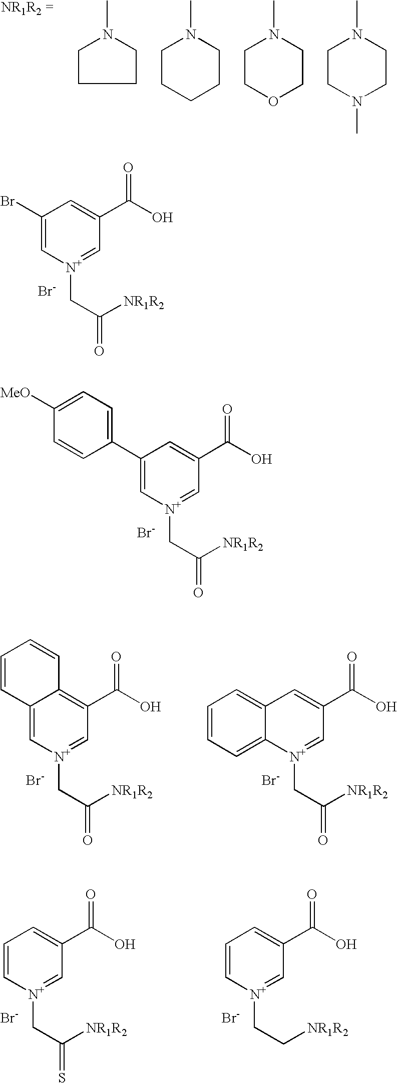
R₁═H, Me, Et
R₂═C5-C9 hydrocarbyl
or
R₂=—(CH₂)ₙ NR₁R₂,
Where n=5-9 and 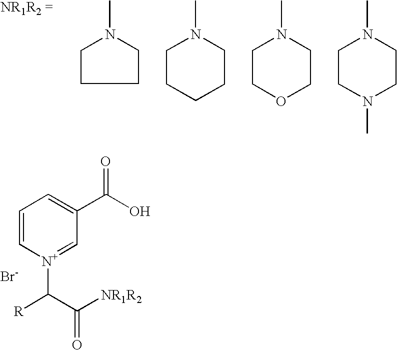
R₁ ═H, Me, Et
R₂═C5-C9 hydrocarbyl
or
R₂=—(CH₂)ₙNR₁R₂,
Where n=5-9 and

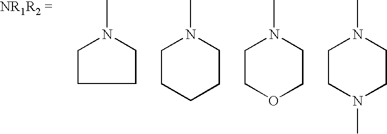

Compounds also include branched compounds where R is hydrocarbyl or oxyhydrocarbyl.

Examples of these compounds have already been described herein. Another example of a preferred compound of formula (C) is that of formula (E) (as shown below) or a pharmaceutically acceptable salt thereof or a mimetic thereof or a bioisotere thereof. Sometimes the compound of formula (E) is referred to as “BZ320”. Another example of a preferred compound of formula (C) is that of formula (F) (as shown below) or a pharmaceutically acceptable salt thereof or a mimetic thereof or a bioisotere thereof. Sometimes the compound of formula (F) is referred to as “BZ321”. The term “mimetic” relates to any chemical which has the same qualitative activity or effect as a reference agent. 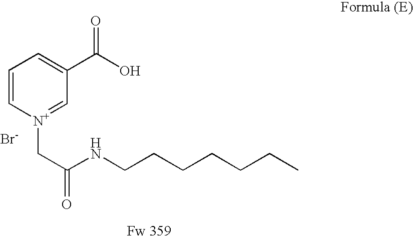 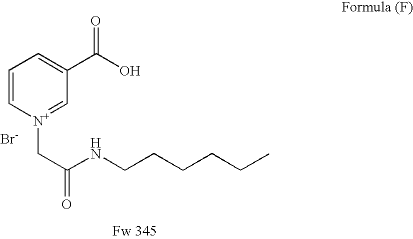
Synthesis Details for BZ320 and BZ321.
Materials and Methods

Thin layer chromatography (TLC) was performed on precoated plates (Merck TLC aluminum sheets silica 60 F254) with eluants as indicated. The compounds were detected using a UV lamp at 254 nm. Flash chromatography was carried out using Sorbisil C60 silica gel. Reagents were obtained from commercial sources and used without further purification unless otherwise stated.

¹H and ¹³C NMR spectra were recorded on either a JEOL GX270 or Varian 400 MHz spectrometer. Coupling constants, J, are measured in Hertz (Hz) and the following abbreviations have been used: s, singlet; d, doublet; t, triplet; q, quartet; m, multiplet; br, broad. Unless otherwise stated chemical shifts were measured in parts per million (ppm) relative to residual protonated solvent.

Melting points were determined using a Reichert Jung Thermo Galen Kofler block and are uncorrected. Mass spectra were recorded using positive and negative fast atom bombardment (FAB) with 3-nitro benzyl alcohol (NBA) as a matrix carrier.
Synthesis of the Pyridinium Salts

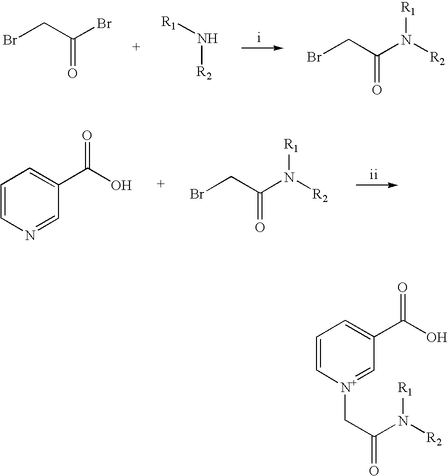

Synthesis of pyridinium salts: i) Ether, −10° C., 0.5 h; ii) DMF, 60° C., overnight in the dark.

General Procedure for Preparing Bromoacetylamides.

Amine (3 ml, 24 mmol) was added dropwise to a stirring solution of 2-bromo-acetylbromide (10 ml, 12 mmol) at −10° C. The temperature of the reaction mixture was kept below 0° C. to avoid any possible side reactions. When the reaction was complete, cold water was added to dissolve the precipitated inorganic salt. The organic layer was separated and washed successively with aqueous acetic acid (20 ml, 1M), aqueous NaOH (20 ml, 1M) and finally with saturated brine. After removal of the ether, the crude product was used for alkylation directly without further purification. Analytical data was obtained after recrystallization or flash chromatography.

General Procedure for Alkylation Reaction

1 equivalent of nicotinic acid (or a derivative thereof prepared by methods known to those versed in the art) and 1 equivalent of 2-bromoacetylamide is dissolved in dry DMF (4 ml). The reaction mixture is then heated at 60° C. for 12 h in dark. After removal of the DMF by evaporation under reduced pressure, the product is obtained as an oil after flash chromatography or is crystallized from methanol/ether.

### 3-Carboxy-1-hexylcarbamoylmethyl-pyridinium (BZ321)& 3-Carboxy-1-heptylcarbamoylmethyl-pyridinium (BZ320)

Procedure for C7 analogue (BZ320) and C6 analogue (BZ321): To a solution of nicotinic acid (3 g, 24.36 mmol) in DMF (55 mL) was added bromoacetamide (24.36 mmol). The resulting solution was heated at 67° C. for overnight. DMF was evaporated and the white residue was redissolved in small amount of MeOH (ca 5 mL). Addition of ether gave crude products as white precipitate. The crude products were further purified by crystallisation in MeOH/acetone.

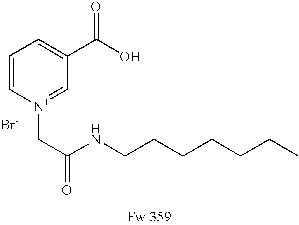

(BZ320, C7 analogue) MP: ¹H NMR (D₂O, 270 MHz) δ 9.30 (s, 1H, H-2), 9.04 (d, J_{6,5} 8.1 Hz, 1H, H-6), 8.92 (d, J_{4,5} 7.2 Hz, 1H, H-4), 8.20 (dd, J_{5,4} 7.2 Hz and J_{5,6} 8.1 Hz, 1H, H-5), 5.49 (s, 2H, CH₂CO), 3.22 (m, 2H, CH₂N), 1.49 (m, 2H, CH₂), 1.23 (m, 8H, 4CH₂), 0.79 (m, 3H, CH₃); ¹³C NMR (D₂O, 67.5 MHz) δ 165.21, 148.33, 147.19, 146.89, 132.03, 128.23, 62.13, 40.10, 31.12, 28.22, 28.09, 26.04, 22.05, 13.47;

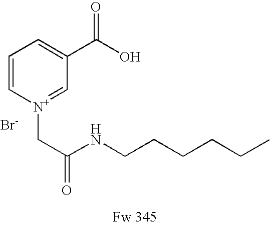

(BZ321, C6 analogue) MP: ¹H NMR (D₂O, 270 MHz) δ 9.30 (s, 1H, H-2), 9.04 (d, J_{6,5} 8.1 Hz, 1H, H-6), 8.92 (d, J_{4,5} 6.2 Hz, 1H, H-4), 8.20 (dd, J_{5,4} 6.2 Hz and J_{5,6} 8.1 Hz, 1H, H-5), 5.49 (s, 2H, CH₂CO), 3.22 (m, 2H, CH₂N), 1.49 (m, 2H, CH₂), 1.24 (m, 6H, 3CH₂), 0.80 (CH₃); ¹³C NMR (D₂O, 67.5 MHz) δ 165.25, 148.16, 147.12, 146.83, 132.77, 128.19, 62.11, 40.12, 30.73, 28.20, 25.79, 22.03, 13.41).

Other Compounds of Formula (C)

Other compounds of Formula (C) may be prepared in the same manner as BZ320 or BZ321. For example, if either of R1 or R2 is of a different alkyl chain length, then the same synthesis protocol as described above is used except that nicotinic acid is reacted with an appropriate different alkylating reagent (i.e. a reagent that provides the required alkyl chain length).

### Example Section Four

Part A

Reference is made in this section to FIGS. 17-22 which present graphs and charts.

In detail:

FIG. 17 shows the effect of NAADP antagonist BZ194 on Ca²⁺ signalling in rat MBP-specific T cells. Resting rat T cells were loaded with fura2/AM and suspension measurements were carried out as described in [4]. Arrows indicate time point of addition of antibody or Ca²⁺ solution. A, Addition of anti-CD3-mAb (1^{st} arrow) and a crosslinking, secondary antibody (2^{nd} arrow). B, Bar chart summarizing data from A (transient Ca²⁺ peak and Ca²⁺ plateau; mean±s.e.m, n=8-9). C, Under Ca²⁺ free conditions the anti-CD3-mAb (1^{st} arrow) and secondary crosslinking antiserum (2^{nd} arrow) were added; then [Ca²⁺] was increased to 1 mM (3^{rd} arrow). D, Bar chart summarizing data from C (transient Ca²⁺ release peak, Ca²⁺ influx peak, Ca²⁺ influx plateau and the difference between Ca²⁺ influx peak and plateau (=Overshoot); mean±s.e.m, n=6-7). The negative controls are without stimulation by anti-CD3-mAb.

FIG. 18 shows the effect of BZ194 on in vitro-cultured GFP+Rat T cells. Cells were stimulated in presence of MBP-pulsed irradiated APCs and increasing concentrations of BZ194. After 48 h cells were collected for cytofluorometric cell count A and RNA extraction was performed for quantitative PCR analysis (IL2) B. Effect of BZ194 on pre-activated T cell blasts C. Amplification of MBP-specific T cells which had been incubated before in the presence of DMSO or 0.5 mM or 1 mM BZ194 D.

FIG. 19 shows cell distribution in spleen and CNS after BZ194 treatment.

Cytofluorometric cell counts of MBP-specific GFP T cells in spleen A and CNS B of BZ194 (500 μmol) and DMSO-treated animals at d 3 after T cell transfer.

FIG. 20 shows motility of T_{MBP-GFP} cells in the spleen after BZ194 treatment.

Intravital videomicroscopy of the spleen of vehicle (DMSO, left) A or BZ194 B-treated animals 3 d after T cell transfer. The ratio between stationary and motile MBP-specific GFP T cells (upper graphs) and cell trajectories (lower graphs, 10 min analysis) are shown. Stationary cells are defined as cells moving less than 10 μm in 10 minutes.

FIG. 21 shows cytokine production is down-regulated in BZ194 treated EAE animals.

Quantitative PCR of spinal cords and spleens 4 days after T cell transfer of animals treated with vehicle (DMSO) of BZ 194 (5 d, 500 μmol). IFNg A,B and IL-2 C,D mRNAs were measured in relation to housekeeping gene β actin A,C or T cell marker CD3 B,D.

FIG. 22 shows recruitment of inflammatory cells after BZ194 treatment.

Cytofluorometric counts of T cells (CD4/8, abTCR), monocytes/macrophages (CD11b), dendritic cells (CD11c), B cells (OX33/CD45RA) and LFA-1+ (CD11a) and MHC class II+ (OX-6) cells in EAE lesions 4 days after T cell transfer after BZ194 (5 d, 500 μmol, blue bars) or vehicle (DMSO, magenta bars).

Part B

Activation and Ca²⁺ Signalling of Encephalitogenic T Cells in MS and EAE

Autoimmune diseases of the CNS are initiated by CD4+, brain-specific T lymphocytes. These autoreactive T cells are normal components of the immune system [1]. Upon activation by specific antigen, superantigens, or by cross-reacting microbial antigens these encephalitogenic T cells invade the CNS via the endothelial blood-brain barrier (BBB) to initiate disease [2-4]. This scenario emerged from studies of experimental autoimmune encephalomyelitis (EAE) and was later extended to other organ specific autoimmune diseases. Commonly, EAE is induced in susceptible animals by immunization with brain antigens (active EAE) or by transfer of brain antigen-specific CD4+ T cells [5]. In the classical adoptive transfer EAE (t-EAE) of the Lewis rat, T lymphocytes directed against myelin basic protein (MBP) mediate severe paralytic disease associated with intense CNS inflammation. The properties of this tEAE model allow us to concentrate on the pathogenic potential of autoreactive T cells.

Importantly, tEAE can be exclusively evoked by transfer of activated T cells [5]. The clinical course after transfer of these activated T cell blasts is characterized by unrivalled reproducibility and predictability [6]. The disease is monophasic and self-limited. The pathology is strictly dependent on the transferred T cells and monocytes/macrophages. CNS inflammation and clinical disease occur after a time period of three to four days following T cell transfer. The migration pattern and functional properties of the autoreactive T cells in this preclinical period of tEAE have been recently analyzed. Thereby, it was found, that autoreactive T cells have to be reactivated within the target organ in order to induce clinical disease [8].

Thus, the activation and re-activation processes of T cells are central check points which decide about onset and/or progression of autoimmune diseases of the CNS. A number of molecular mechanisms is involved in T cell activation [reviewed in 10]; one of the essential intracellular mechanisms is the elevation of the free cytosolic and nuclear calcium concentration ([Ca^{2+]}ᵢ; reviewed in 11, 12). Ca²⁺ signalling describes the fact that upon stimulation of receptors in the plasma membrane [Ca²⁺]ᵢ undergoes changes. Usually, a global increase in [Ca²⁺]ᵢ is preceded by subcellular pacemaker Ca²⁺ signals. Both local and global Ca²⁺ signals often exhibit complex spatio-temporal patterns, e.g. oscillations and/or waves. [Ca²⁺]ᵢ is the resultant from at least four individual processes: Ca²⁺ release from intracellular stores and Ca²⁺ inflow from the extracellular space elevate [Ca²⁺]ᵢ, while Ca²⁺ extrusion by calcium pumps in both intracellular and plasma membranes and binding of Ca²⁺ ions to Ca²⁺ binding proteins reduces [Ca²⁺]ᵢ.

Ca²⁺ signalling of encephalitogenic T cells in multiple sclerosis or EAE is either activated specifically by antigenic peptide via the T cell receptor/CD3 complex (TCR/CD3; ref. 13) or unspecifically by proinflammatory cytokines, e.g. interferon-γ (IFNγ; ref. 14), or a combination of interleukin 2 (IL-2), interleukin-6 (IL-6) and tumor necrosis factor-α (TNFα; ref. 15). Among the downstream transducers of increased [Ca²⁺]ᵢ are calcium/calmodulin-dependent kinase II [16], calpain [17], and Ca²⁺-activated K⁺-channels [18]. Activation of these proteins has been linked to transendothelial migration across the BBB [16] and to proliferation of T cells. Accordingly, inhibitors of some of these Ca²⁺ activated downstream transducers were successfully used to treat the symptoms in EAE [18]. These examples demonstrate that the mechanisms underlying Ca²⁺ signalling in encephalitogenic T cells are good candidates for new pharmacological approaches.

### REFERENCES

1. H. Wekerle, M. Bradl, C. Linington, G. K\ddot{aa}b, and K. Kojima. 1996. The shaping of the brain-specific T lymphocyte repertoire in the thymus. Immunol. Rev. 149:231-243.
2. H. Wekerle, C. Linington, H. Lassmann, and R. Meyermann, Cellular immune reactivity within the CNS. Trends Neurosci. 9:271-277 (1986)
3. A. H. Cross, Cannella, B., Brosnan, C. F., C. S. Raine, Hypothesis: Antigen-specific T cells prime central nervous system endothelium for recruitment of nonspecific inflammatory cells to effect autoimmune demyelination. J. Neuroimmunol. 33, 237-244 (1991)
4. L. Steinman, A few autoreactive cells in an autoimmune infiltrate control a vast population of nonspecific cells: A tale of smart bombs and the infantry. Proc. Nat. Acad. Sci. USA 93, 2253-2256 (1996)
5. A. Ben-Nun, H. Wekerle, and I. R. Cohen, The rapid isolation of clonable antigen-specific T lymphocyte lines capable of mediating autoimmune encephalomyelitis. Eur. J. Immunol. 11:195-199 (1981)
6. H. Wekerle, K. Kojima, J. Lannes-Vieira, H. Lassmann, and C. Linington, Animal models. Ann. Neurol. 36:S47-S53 (1994)
7. H. Lassmann, and H. Wekerle, Experimental models of multiple sclerosis. In McAlpine's Multiple Sclerosis. A. Compston, G. Ebers, H. Lassmann, B. Matthews, and H. Wekerle, eds. Churchill Livingston, London, pp. 409-434 (1998)
8. A. Flügel, T. Berkowicz, T. Ritter, M. Labeur, D. Jenne, Z. Li, J. Ellwart, M. Willem, H. Lassmann, H. Wekerle, Migratory activity and functional changes of green fluorescent effector T cells before and during experimental autoimmune encephalomyelitis, Immunity 14:547-560 (2001)
9. Berridge M J. Lymphocyte activation in health and disease. Crit. Rev. Immunol. 1997; 17:155-78.
10. Guse A H. Ca²⁺ signalling in T-lymphocytes. Crit. Rev. Immunol. 1998; 18:419-48
11. Lewis R S. Calcium signalling mechanisms in T lymphocytes. Annu. Rev. Immunol. 2001; 19:497-521.
12. Wang C, Mooney J L, Meza-Romero R, Chou Y K, Huan J, Vandenbark A A, Offner H, Burrows G G. Recombinant TCR ligand induces early TCR signalling and a unique pattern of downstream activation. J. Immunol. 2003; 171:1934-40.
13. Martino G, Clementi E, Brambilla E, Moiola L, Comi G, Meldolesi J, Grimaldi L M. Gamma interferon activates a previously undescribed Ca²⁺ influx in T lymphocytes from patients with multiple sclerosis. Proc. Natl. Acad. Sci. USA. 1994; 91:4825-9.
14. Martino G, Grohovaz F, Brambilla E, Codazzi F, Consiglio A, Clementi E, Filippi M, Comi G, Grimaldi L M. Proinflammatory cytokines regulate antigen-independent T-cell activation by two separate calcium-signalling pathways in multiple sclerosis patients. Ann Neurol. 1998; 43:340-9.
15. Poggi A, Zocchi M R, Carosio R, Ferrero E, Angelini D F, Galgani S, Caramia M D, Bernardi G, Borsellino G, Battistini L. Transendothelial migratory pathways of V delta 1+TCR gamma delta+ and V delta 2+TCR gamma delta+T lymphocytes from healthy donors and multiple sclerosis patients: involvement of phosphatidylinositol 3 kinase and calcium calmodulin-dependent kinase II. J. Immunol. 2002; 168:6071-7.
16. Shields D C, Schaecher K E, Goust J M, Banik N L. Calpain activity and expression are increased in splenic inflammatory cells associated with experimental allergic encephalomyelitis. J. Neuroimmunol. 1999; 99:1-12.
17. Wulff H, Calabresi P A, Allie R, Yun S, Pennington M, Beeton C, Chandy K G. The voltage-gated Kv1.3 K(+) channel in effector memory T cells as new target for MS. J. Clin. Invest. 2003; 111:1703-13.
18. Beeton C, Barbaria J, Giraud P, Devaux J, Benoliel A M, Gola M, Sabatier J M, Bernard D, Crest M, Beraud E. Selective blocking of voltage-gated K⁺ channels improves experimental autoimmune encephalomyelitis and inhibits T cell activation. J. Immunol. 2001; 166:936-44.
19. H. Wekerle, M. Bradl, C. Linington, G. K\ddot{aa}b, and K. Kojima. 1996. The shaping of the brain-specific T lymphocyte repertoire in the thymus. Immunol. Rev. 149:231-243.
   Part C
   Methods
   Cell Culture—Jurkat T-Lymphocytes (Clone JMP) were Cultured in RPMI 1640 Supplemented with Glutamax I, 10% (v/v) Newborn or Fetal Calf Serum, 25 mM HEPES, 100 units/ml Penicillin and 50 μg/ml Streptomycin.

Ratiometric Ca² imaging—The cells were loaded with fura-2/AM and kept in the dark at room temperature until use. Thin glass coverslips (0.1 mm) were coated with BSA (5 mg/ml), and poly-L-lysine (0.1 mg/ml). Silicon grease was used to seal small chambers consisting of a rubber O-ring on the glass coverslips. Then, 60 μl buffer A containing 140 mM NaCl, 5 mM KCl, 1 mM MgSO₄, 1 mM CaCl₂, 1 mM NaH₂PO₄, 5.5 mM glucose, and 20 mM HEPES (pH 7.4), and 40 μl cell suspension (2*10⁶ cells/ml) suspended in the same buffer were added into the small chamber. The coverslip with cells slightly attached to the BSA/poly-L-lysine coating was mounted on the stage of a fluorescence microscope (Leica DM IRE2).

Ratiometric Ca²⁺ imaging was performed using an Improvision imaging system (Heidelberg, Germany) built around the Leica microscope at 100-fold magnification. Illumination at 340 and 380 nm was carried out using a monochromator system (Polychromator IV, TILL Photonics, Gräfelfing, Germany). Images were taken with a gray-scale CCD camera (type C4742-95-12ER; Hamamatsu, Enfield, United Kingdom; operated in 8-bit mode). The spatial resolution was 512×640 pixel at 100-fold magnification. Camera exposure times were 12 msec (at 340 nm) and 4 msec (at 380 nm). The acquisition rate was approx. 1 ratio/160 msec. Raw data images were stored on hard disk. Confocal Ca²⁺ images were obtained by off-line no-neighbor deconvolution using the volume deconvolution module of the Openlab software. The deconvolved images were used to construct ratio images (340/380). Finally, ratio values were converted to Ca²⁺ concentrations by external calibration. To reduce noise, ratio images were subjected to median filter (3×3). Data processing was performed using Openlab software v 1.7.8, v3.0.9 or v3.5.2 (Improvision, Tübingen, Germany).

Microinjection—Microinjections were carried out using an Eppendorf system (transjector type 5246, micromanipulator type 5171, Eppendorf-Netheler-Hinz, Hamburg, Germany) with Femtotips II as pipettes. NAADP and/or compounds were diluted to their final concentration in intracellular buffer (20 mM HEPES, 110 mM KCl, 2 mM MgCl₂, 5 mM KH₂PO₄, 10 mM NaCl, pH 7.2) and filtered (0.2 μm) before use. To avoid any contamination of Ca²⁺ in the solution to be injected, a small amount of Chelex resin beads was added. Injections were made using the semiautomatic mode of the system with the following instrumental settings: injection pressure 60 hPa, compensatory pressure 30 hPa, injection time 0.5 s, and velocity of the pipette 700 μn/s. Under such conditions the injection volume was 1-1.5% of the cell volume.

Measurement of [Ca²⁺]ᵢ in Cell Suspensions in Intact Cells

[Ca²⁺]ᵢ was measured by using the fluorescent indicator Fura2/AM. In brief, 10⁷ cells were loaded with Fura2/AM, and fluorescence was determined in batches of 1.5×10⁶ cells with an Hitachi F-2000 fluorimeter at excitation and emission wavelengths of 340 and 380 nm (exc.) and 505 nm (em.), respectively. Measurements were carried out at 20° C. At the end of each single measurement maximal and minimal ratios were determined by addition of Triton X-100 and EGTA/Tris.

Quantitative PCR.

mRNA was extracted using standard protocols (Sigma-Aldrich) and reversed to cDNA (Invitrogen). Taqman analysis was performed as reported (Flügel et al. 2001, Immunity) using ABI Prim 7700 Sequence Detector “Taqman” (PE Applied Biosystems). The expression of a housekeeping gene (b-actin) was set into relation to the specific mRNA. Data were obtained by independent duplicate measurements. The CT value of the individual measurements did not exceed 0.5 amplification cycles.

Cell Isolation, Cytofluorometry and Fluorescence Activated Sorting (FACS).

Single cell suspensions from organs were prepared as described previously (Flügel et al. 2001, Immunity). T_{GFP} cells were sorted immediately after cell preparation in PBS containing 2% glucose at 4° C. using FACS Vantage (Becton Dickinson) or MoFlo sorter (Cytomation Bioinstruments, Freiburg, Germany). Cytofluorometric analysis was performed with FACS-Calibur operated by Cell Quest software (Becton Dickinson). The following monoclonal antibodies were used for surface membrane analysis: W3/25 (anti-CD4), OX33 (CD45RA, B cells, both from Serotec, Düsseldorf, Germany), R73 (abTCR), OX-6 (rat MHC class II), OX-40 antigen (CD134), OX-39 (CD25, IL-2Ra chain), CD8a, CD8b, CD11a (LFA-1a), CD11b (integrin a_{M} chain), CD11b/c (OX42), CD11c (integrin a_{X} chain) (all Becton Dickinson, Heidelberg, Germany). Allophycocyanin-labeled anti-mouse antibody (Invitrogen, Karlsruhe, Germany) was used as secondary antibody.

Surgical Procedure and Intravital Imaging Settings.

Animals were anesthetized by intraperitoneal injection of xylazine/ketamine (10 mg/kg and 50 mg/kg, respectively), with reapplied doses (1 mg/kg xylazine/5 mg/kg ketamine) administered as required. Adequate oxygenation was ensured by an oxygen (95% O2/5% CO2) mask fixed on the animal's snout. Body temperature was controlled by a heated pad. For spleen imaging temperature and moistness controlled chambers were constructed for inverted microscope settings. Surgically exposed spleens with intact vasculature were carefully placed into the chambers superfused with carbogenated HBSS (95% O₂5% CO₂).

Fluorescence Videomicroscopy:

Time-lapse recordings were performed using an inverted microscope (Zeiss, Axiovert 200M) equipped with a 20×, 0.4 NA objective (Zeiss). Images were acquired using a Coolsnap-HQ camera (Photometrics, Roper Scientific, Tuscon, Ariz.) in 30 s time intervals and processed by MetaMorph (Visitron Systems, Puchheim, Germany). Image J software (freeware, provided by Wayne Rasband, NIH) was used to evaluate cell trajectories and velocity.

Proliferation Assays.

T_{MBP-GFP} cells were co-cultured for 48 h in 96well plates (in DMEM 1% rat serum) with APCs in presence of specific antigen (10 mg/ml MBP) or without. Amplification of T_{MBP-GFP} cells was measured by cytofluorometry as described (Kawakami et al. 2005, J.I.). Their numbers were determined in relation to a known absolute number of added phycoerythrin-labeled plastic beads (Becton-Dickinson). The amplification rate was calculated in relation to the GFP+T cell numbers at day 0. Alternatively, [³H]dT (2 Ci/mmol; Amersham-Buchler) incorporation was used to evaluate proliferation. The radioactive label present in the different cultures was determined as described (Flügel et al., 2001, Immunity)

### REFERENCES

Kawakami N, Odoardi F, Ziemssen T, Bradl M, Ritter T, Neuhaus O, Lassmann H, Wekerle H, Flügel A (2005). Autoimmune CD4+ T cell memory: Life long persistence of encephalitogenic T cell clones in healthy immune repertoires. Journal of Immunology, Jul 1; 175(1):69-81.
Flügel A, Berkowicz T, Ritter T, Labeur M, Li Z, Ellwart J W, Jenne D, Willem M, Lassmann H, and Wekerle H (2001). Migratory activity and functional changes of genetically engineered green fluorescent effector T cells before and during experimental autoimmune encephalomyelitis. Immunity 14: 547-560.
   Part D

In our studies on the compounds of the present invention we found that BZ194 worked well in intact cells. BZ194 blocked TCR/CD3 complex mediated Ca²⁺ signalling in MBP-specific rat T cell suspensions in both our standard protocol (FIG. 17 A,B) and a Ca²⁺-free/Ca²⁺-reintroduction protocol (FIG. 17 C,D). As can be seen in FIG. 17 both the initial Ca²⁺ peak and the sustained Ca²⁺ plateau phase are sensitive to BZ194, and thus appear to be regulated by NAADP.

The initial Ca²⁺ peak has been thought to be mainly controlled by InSP₃, but according to our novel data NAADP appears to play an important role in this phase, too. This result was recently backed up by the first direct measurements of cellular NAADP concentrations in human Jurkat T cells: we showed a rapid rise of NAADP within 10 to 20 sec post TCR/CD3 stimulation [4]. In addition, to its role in initiating the T cell Ca²⁺ response, NAADP appears to be a key player for the sustained Ca²⁺ signalling phase. In both our protocols, BZ194 almost completely abolished sustained Ca²⁺ signalling (FIG. 17 A to D), the Ca²⁺ signalling phase most important for proliferation of T cells. Again, this finding is backed up by direct measurements of NAADP in Jurkat T cells [4]: besides the rapid increase of NAADP within the first 10 to 20 s, a prolonged increase of NAADP between 5 min and at least 20 min was observed [4].

BZ194 strongly influenced the function of rat MBP-specific T cells in vitro and in vivo. Proliferation and cytokine expression of the T cells was tested in vitro after incubation with irradiated MBP-pulsed thymic antigen presenting cells (in rats the commonly used APC type, ref. 5). BZ194 dose dependently suppressed T cell proliferation (FIG. 18A). Additionally, the expression of cytokine production by the T cells was reduced (FIG. 18B). Importantly, these effects were not due to toxicity of BZ194. Amplification of pre-activated MBP-specific T cells was not influenced in the presence of BZ194 (FIG. 18C). Further, foregoing incubation with BZ194 did not block the proliferative response towards MBP challenge (FIG. 18D).

Limited solubility of BZ194 excluded intravenous application due to embolic complications. Intraperitoneal injection of BZ194 solubilized in DMSO (180 mg/kg, treatment for 5 days) was well tolerated by Lewis rats, as tested by inspection, weight development, and immune cell count (not shown). Higher amounts of BZ194 occasionally induced peritonitis, which could be, however, widely avoided by prior filtration of the substance.

In order to analyze the effect of BZ194 on encephalitogenic T cells during the effector phase of EAE we used retrovirally transduced MBP-specific T cells expressing the marker gene green fluorescent protein (T_{MBP-GFP} cells) [6]. Treatment with BZ194 significantly ameliorated clinical EAE induced by intravenous transfer of 5×10⁶ T_{MBP-GFP} cells (Table 4.1).

TABLE 4.1
The NAADP antagonist BZ194 ameliorates tEAE.

| | | Disease | | Max. | Max. body |
|---|---|---|---|---|---|
| | Ani- | onset | Disease | clinical | weight |
| Experiment | mals | (hours p.t.) | duration | score | loss (%) |
| Experiment I | | | | | |
| BZ194 | 5 | 78 | Up to 144 h | 1.4 ± 0.8 | 9 ± 4.3 |
| treated | | | p.t. | | |
| animals | | | | | |
| DMSO | 5 | 68 | Up to 164 h | 3 ± 0.5 | 17 ± 4.7 |
| treated | | | p.t | | |
| animals | | | | | |
| Experiment | | | | | |
| II | | | | | |
| BZ194 | 3 | no | — | — | 8 ± 1.6 |
| treated | | | | | |
| animals | | | | | |
| DMSO | 3 | 68 | Up to 123 h | 1.5 ± 0.3 | 10 ± 1.5 |
| treated | | | p.t | | |
| animals | | | | | |
| Experiment | | | | | |
| III | | | | | |
| BZ194 | 7 | 81 | Up to 129 h | 1.6 ± 0.5 | 10 ± 4.4 |
| treated | | | p.t | | |
| animals | | | | | |
| DMSO | 7 | 72 | Up to 144 h | 3.2 ± 0.2 | 13 ± 2.1 |
| treated | | | p.t | | |
| animals | | | | | |

Animals were treated for 5 d intraperitoneally with 500 μmol BZ194 or vehicle (DMSO).

Mean±SD. Three independent experiments.

T_{MBP-GFP} cells in spleen and lymph nodes (LNs) of BZ194 animals assumed a “migratory phenotype” with up-regulation of MHC class II and down-regulation of activation markers OX-40 and IL-2 receptor (not shown). These changes were recently found to precede their infiltration into the CNS [7]. However, cytofluorometric analysis of spinal cord tissue three days after T cell transfer, i.e. during early clinical EAE indicated that BZ194 seemed to interfere with the capacity of T_{MBP-GFP} cells to invade the CNS. Instead, the cells accumulated in the spleen of the treated animals (FIG. 19A,B). Preliminary results using intravital imaging techniques indicate that the motility of T_{MBP-GFP} cells in the spleen was strikingly changed after BZ194 treatment. The average velocity of T_{MBP-GFP} cells was strongly reduced (3.5 μm/min compared to 6.5 μm/min in DMSO-treated controls, FIG. 20A,B) and the cells tended to form clusters in certain locations (not shown). However, T_{MBP-GFP} cells remained viable and fully reactive: after ex vivo isolation they strongly proliferated upon challenge with MBP (not shown).

The activation levels of T_{MBP-GFP} cells within the target organ were markedly decreased. Quantitative PCR analysis of spinal cord tissue 4 days after T cell transfer showed a significant reduction of cytokine production after BZ194 treatment as indicated by decrease of IL-2, IFNγ (FIG. 21A,C), TNFα, and IL-2 receptor mRNA (not shown) in relation to housekeeping gene β actin as well as CD3 (FIG. 21B,D). Consecutively, the recruitment of T cells, B cells, macrophages and dendritic cells into the CNS after BZ194 treatment was clearly diminished.

We set up two-photon and video recording technologies which will enable us to visualize Ca²⁺ fluxes of T cells in vivo and to analyze the effects of Ca²⁺ antagonists on encephalitogenic T cells in living animals in various locations such as the spleen, LNs, and the spinal cord [8]. Further, we succeeded to establish a rat model with GFP-expression in encephalitogenic memory T cells [9]. Thus, we now are able to examine the role of Ca²⁺ antagonists during the priming phase of EAE.

### REFERENCES

1. Dammernann W, Guse A H. Functional ryanodine receptor expression is required for NAADP-mediated local Ca²⁺ signalling in T-lymphocytes. J Biol. Chem. 2005; 280:21394-9
2. I. Berg, B. V. L. Potter, G. W. Mayr, A. H. Guse, Nicotinic acid adenine dinucleotide phosphate (NAADP⁺) is an essential regulator of T-lymphocyte Ca²⁺-signalling, J. Cell. Biol. 150, 581-588 (2000).

3. Langhorst M F, Schwarzmann N, Guse A H. Ca²⁺ release via ryanodine receptors and Ca²⁺ entry: major mechanisms in NAADP-mediated Ca²⁺ signalling in T-lymphocytes. Cell Signal. 2004; 16(11):1283-9.
4. Gasser A, Bruhn S, Guse A H. Second messenger function of nicotinic acid adenine dinucleotide phosphate (NAADP) revealed by an improved enzymatic cycling assay. J Biol. Chem. 2006 Apr 20; [Epub ahead of print]
5. Ben-Nun, A et al., Eur. J. Immunol. 1981
6. Flügel A, Willem M, Berkowicz T, Wekerle H. Gene transfer into CD4+ T lymphocytes: green fluorescent protein-engineered, encephalitogenic T cells illuminate brain autoimmune responses. Nat. Med. 1999; 5(7):843-7.
7. Flügel A, Berkowicz T, Ritter T, Labeur M, Jenne D E, Li Z, Ellwart J W, Willem M, Lassmann H, Wekerle H. Migratory activity and functional changes of green fluorescent effector cells before and during experimental autoimmune encephalomyelitis. Immunity. 2001; 14(5):547-60
8. Kawakami N, Nagerl U V, Odoardi F, Bonhoeffer T, Wekerle H, Flügel A. Live imaging of effector cell trafficking and autoantigen recognition within the unfolding autoimmune encephalomyelitis lesion. J Exp Med. 2005; 201(11):1805-14.
9. Kawakami N, Odoardi F, Ziemssen T, Bradl M, Ritter T, Neuhaus 0, Lassmann H, Wekerle H, Flügel A. Autoimmune CD4+ T cell memory: lifelong persistence of encephalitogenic T cell clones in healthy immune repertoires. J. Immunol. 2005; 175(1):69-81
10. Wagner G K, Guse A H, Potter B V L. Rapid synthetic route toward structurally modified derivatives of cyclic adenosine 5′-diphosphate ribose. J Org Chem. 2005; 70(12):4810-9.
11. Gu X, Yang Z, Zhang L, Kunerth S, Fliegert R, Weber K, Guse A H, Zhang L. Synthesis and biological evaluation of novel membrane-permeant cyclic ADP-ribose mimics: N1-[(5″-O-phosphorylethoxy)methyl]-5′-O-phosphorylinosine 5′,5″-cyclicpyrophosphate (cIDPRE) and 8-substituted derivatives. J Med. Chem. 2004; 47(23):5674-82.
12. Gasser A, Glassmeier G, Fliegert R, Langhorst M F, Meinke S, Hein D, Kruger S, Weber K, Heiner I, Oppenheimer N, Schwarz J R, Guse A H. Activation of T cell calcium influx by the second messenger ADP-ribose. J Biol. Chem. 2006; 281(5):2489-96
13. Gasser A, Guse A H. Determination of intracellular concentrations of the TRPM2 agonist ADP-ribose by reversed-phase HPLC. J Chromatogr B Analyt Technol Biomed Life Sci. 2005; 821(2):181-7.
   Part E

In T-lymphocytes BZ52 and BZ194, analogues of NAADP mimicking the nicotinic acid headgroup, efficiently inhibited NAADP-mediated Ca²⁺ signalling, TCR/CD3-induced Ca²⁺ signalling and proliferation. Furthermore, a substantial therapeutic effect was observed in the multiple sclerosis model of transfer-EAE in the Lewis rat. Optimizing the pharmacological lead BZ194 can consist of several rounds each comprised of (i) chemical synthesis of derivatives of BZ194, (ii) analysis of the effects of the new analogs towards Ca²⁺ signalling induced by NAADP or induced by TCR/CD3 ligation in vitro, (iii) analysis of the effects of the new analogs towards proliferation and cytokine expression induced by MBP in vitro, (iv) motility assays in vitro and in vivo, and (v) analysis of the effects of the new analogs towards their therapeutic potential in the transfer-EAE model in Lewis rats in vivo.

The immunosuppressive effect of BZ194-type compounds appears to reside in their antagonistic properties of NAADP induced Ca²⁺ release. In vitro we have shown this using cultured encephalitogenic T cells stimulated by ligation of the TCR/CD3 complex using antibodies. A more complex interaction is observed in vivo when T cells are activated by antigen-presenting cells via complex cell-cell interactions. These include, in addition to the MHC-II/antigenic peptide/TCR interaction, also co-stimulatory signalling events which cannot be mimicked sufficiently precisely by just adding specific antibodies. Thus, the complex cell-cell interactions of antigen-presenting cells and encephalitogenic T cells can be mimicked in vitro in confocal Ca²⁺ imaging experiments and the effect of BZ194 (or an optimized version thereof) on Ca²⁺ signalling in T cells observed under these conditions can be analysed. Live imaging of Ca²⁺ signalling events in living CNS tissue and spleen can also be achieved, e.g. by injection of Ca²⁺ fluorochrome-loaded T cells in CNS-slices or intravenously in the presence or absence of NAADP antagonist.

Since BZ194-type compounds may also interfere with metabolism of NAADP, e.g. blockade of NAADP formation, these effects can be studied in vitro.

Part F

The main findings are: (i) an important involvement of the nicotinic acid adenine dinucleotide phosphate (NAADP)/Ca²⁺ signalling pathway in (re)-activation of encephalitogenic T cells, (ii) development and characterization of a new class of immunosuppressive NAADP antagonists, (iii) establishment of intravital imaging technologies which allows to visualize the effect of Ca²⁺ pathway antagonists on T cell motility and function, and (iv) a pivotal role of the adenosine diphosphoribose (ADPR)/TRPM2/Ca²⁺ signalling pathway in induction of apoptosis of T cells.

Optimization of the NAADP antagonist BZ194 can be performed by repeated cycles of chemical derivatization, followed by in vitro screening, detailed in vitro characterization, and finally in in-depth testing of selected compounds both in vitro and in vivo in the multiple sclerosis model of transfer experimental autoimmune encephalomyelitis (transfer-EAE) in rats.

In depth-analysis of the immunosuppressive effects of BZ194-type NAADP antagonists includes confocal calcium imaging of antigen-presenting cell—T cell interactions both in vitro and in vivo by 2-photon intravital microscopy. Furthermore, potential additional effects of NAADP antagonists, e.g. effects on NAADP metabolism or effects on transcription of unrelated genes, will be studied by HPLC methods and microarrays, respectively.

Certain Aspects

Certain aspects of the present invention will now be described by way of numbered paragraphs.
1. The use of a compound of formula (I): 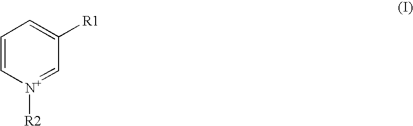
wherein:
R1 comprises a carbonyl group
R2 is a hydrocarbyl group;
   optionally wherein said ring is further substituted;
   or a pharmaceutically acceptable salt thereof;
   in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
   2. Use according to paragraph 1 wherein said compound is cell permeable.
   3. Use according to paragraph 1 or paragraph 2 wherein said compound has a relative molecular mass (RMM) of less than about 500.
   4. Use according to any preceding paragraph wherein R2 is a hydrocarbyl group comprising a carbonyl group.
   5. Use according to any preceding paragraph wherein R2 is a hydrocarbyl group comprising an amide group.
   6. Use according to any preceding paragraph wherein R2 is CH₂C(O)NH₂.
   7. Use according to any preceding paragraph wherein R1 is COOH.
   8. Use according to any preceding paragraph wherein said compound is a mimetic of nicotinic group of NAADP, wherein said NAADP has the formula: 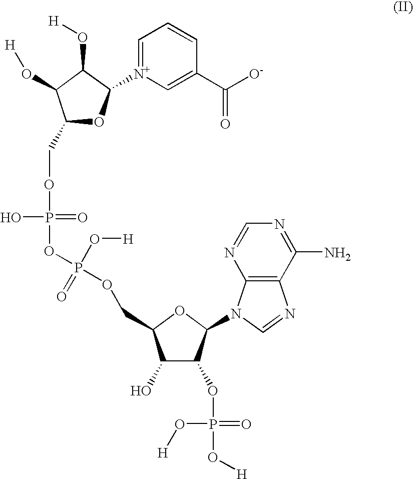
   9. A pharmaceutical composition comprising a compound as defined in any of the preceding paragraphs or a pharmaceutically acceptable salt thereof admixed with a pharmaceutically acceptable carrier, diluent or excipient.
   10. A pharmaceutical composition according to paragraph 9 wherein said composition comprises one or more additional pharmaceutically active compounds.
   11. A compound of formula (I) or a pharmaceutically acceptable salt thereof as defined in any one of paragraphs 1 to 8 for use in medicine.
   12. A compound of formula (I) or a pharmaceutically acceptable salt thereof.
   13. A medicament comprising a compound according to any of paragraphs 1-8.
   14. An assay method for identifying an agent that modulates intracellular calcium release comprising the steps of:
   (a) providing an agent;
   (b) providing an NAADP receptor;
   (c) contacting said agent with an NAADP receptor; and
   (d) measuring the level of intracellular calcium release;
   wherein a difference between (i) the level of intracellular calcium release in the presence of the agent; and (ii) the level of intracellular calcium release in the absence of the agent is indicative of an agent that modulates intracellular calcium release and may be useful in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
   15. An assay method according to paragraph 14 wherein said agent is cell permeable.
   16. An assay method according to paragraph 14 or paragraph 15 wherein said agent has a relative molecular mass (RMM) of less than about 500.
   17. An assay method according to any of paragraphs 14-16 wherein said agent is a mimetic of nicotinic group of NAADP, wherein said NAADP has the formula: 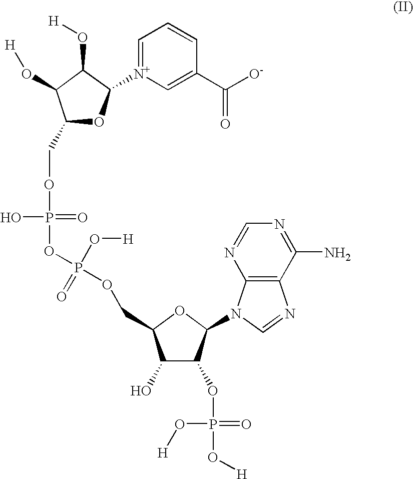
   18. An assay method comprising the steps of:
   (a) performing the assay method according to any of paragraphs 14-17;
   (b) identifying one or more agents capable of modulating intracellular calcium release; and
   (c) preparing a quantity of those one or more identified agents.
   19. A method comprising the steps of:
   (a) performing the assay method according to any of paragraphs 14-17;
   (b) identifying one or more agents capable of modulating intracellular calcium release; and
   (c) preparing a pharmaceutical composition comprising those one or more identified agents.
   20. An agent identifiable, preferably, identified by the assay method according to paragraph any of paragraphs 14-17.
   21. A pharmaceutical composition prepared by the method of paragraph 19.
   22. A method of treating and/or preventing a disease in a human or animal patient in need of same which method comprises administering to the patient an effective amount of a compound as defined in any of paragraphs 1-8 or 11-12, a composition according to paragraphs 9 or 10, or a medicament according to paragraph 13.
   23. A process of preparing a pharmaceutical composition, said process comprising admixing one or more of the compounds defined in any of paragraphs 1-8 or 11-12 with a pharmaceutically acceptable diluent, excipient or carrier.
   24. A pharmaceutical pack comprising one or more compartments, wherein at least one compartment comprises one or more of the compounds defined in any of paragraphs 1-8 or 11-12, a composition according to paragraphs 9 or 10, or a medicament according to paragraph 13.
   25. A container comprising a compound according to any of paragraphs 1-8 or 11-12, a composition according to paragraphs 9 or 10, or a medicament according to paragraph 13, wherein said container is labelled for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
   26. The use of a compound of formula (I) in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
   wherein said compound is cell permeable;
   wherein said compound has a relative molecular mass of less than about 500;
   wherein said compound is a mimetic of the nicotinic group of NAADP, wherein said NAADP has the formula: 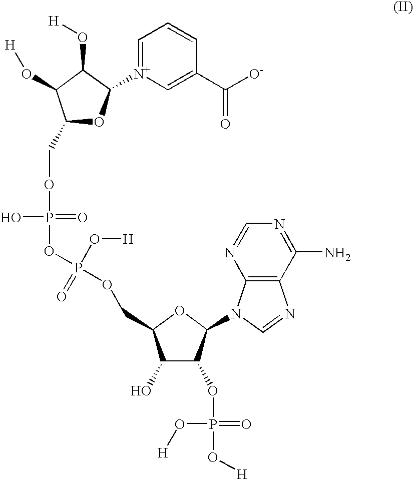
   27. The use of a compound in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
   wherein said compound is cell permeable;
   wherein said compound has a relative molecular mass of less than about 500;
   wherein said compound is a mimetic of the nicotinic group of NAADP, wherein said NAADP has the formula: 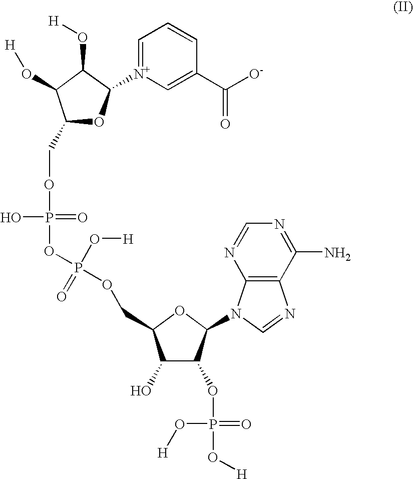
   and wherein said compound has the formula (A): 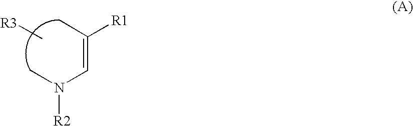
   wherein R1 and R2 are ring substituents;
   wherein R1 and R2 are as defined for compound of formula (I); and
   wherein R3 represents one or more substituents, the or each substituent being independently selected from H, substituted or unsubstituted aryl, C1-20 alkyl, F, Cl, Br, I, OY, NYₙ (n=1, 2, or 3), SY, COY, CONY_{z} (z=2), C(O)OY, wherein the or each Y is independently selected from H, a substituted or unsubstituted aryl, and a C1-20 alkyl group.
   28. A compound for use in medicine
   wherein said compound is cell permeable;
   wherein said compound has a relative molecular mass of less than about 500;
   wherein said compound is a mimetic of the nicotinic group of NAADP, wherein said NAADP has the formula: 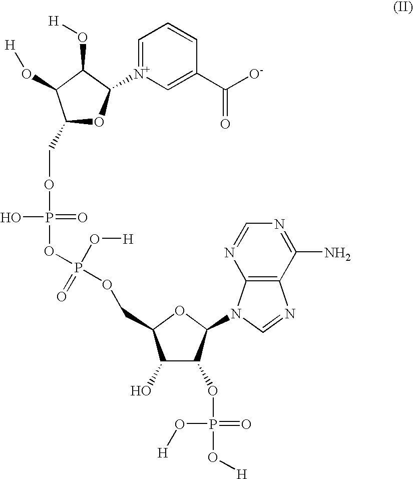
   and wherein said compound has the formula (A): 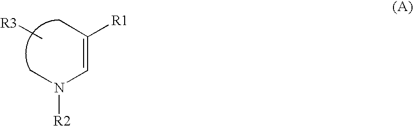
   wherein R1 and R2 are ring substituents;
   wherein R1 and R2 are as defined for compound of formula (I); and
   wherein R3 represents one or more substituents, the or each substituent being independently selected from H, substituted or unsubstituted aryl, C1-20 alkyl, F, Cl, Br, I, OY, NYₙ (n=1, 2, or 3), SY, COY, CONY_{z} (z=2), C(O)OY, wherein the or each Y is independently selected from H, a substituted or unsubstituted aryl, and a C1-20 alkyl group.
   29. A compound, or a pharmaceutical composition comprising same,
   wherein said compound is cell permeable;
   wherein said compound has a relative molecular mass of less than about 500;
   wherein said compound is a mimetic of the nicotinic group of NAADP, wherein said NAADP has the formula: 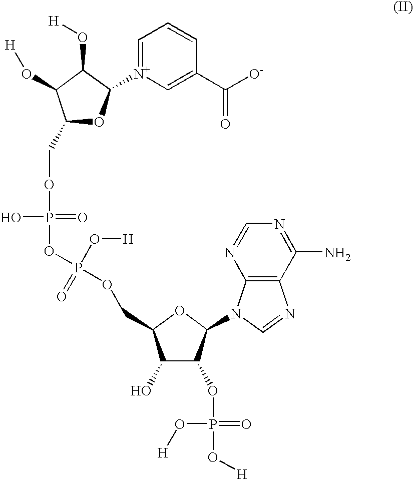
   and wherein said compound has the formula (A): 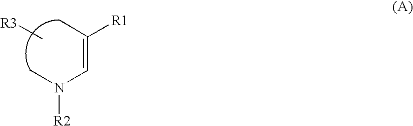
   wherein R1 and R2 are ring substituents;
   wherein R1 and R2 are as defined for compound of formula (I); and
   wherein R3 represents one or more substituents, the or each substituent being independently selected from H, substituted or unsubstituted aryl, C1-20 alkyl, F, Cl, Br, I, OY, NYₙ (n=1, 2, or 3), SY, COY, CONY_{z} (z=2), C(O)OY, wherein the or each Y is independently selected from H, a substituted or unsubstituted aryl, and a C1-20 alkyl group.
   30. The invention according to any one of the preceding paragraphs wherein the compound is of the formula (A1): 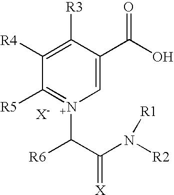
   wherein X (of the ═X group) is O, S, CH2 or (H,H);
   wherein R1 is hydrocarbyl group or an oxyhydrocarbyl group or H;
   wherein R2 is hydrocarbyl group or an oxyhydrocarbyl group or H;
   optionally wherein R1 and R2 can be linked so as to form a hydrocarbyl ring structure
   wherein R3 is H or halo or a hydrocarbyl group;
   wherein R4 is H or halo or a hydrocarbyl group;
   wherein R5 is H or halo or a hydrocarbyl group;
   wherein R6 is H or halo or a hydrocarbyl group;
   wherein X⁻ is an anion, preferably a halo anion, preferably Br—;
   wherein the compound may be salt form or as a Zwitterion.
   31. The invention according to any one of the preceding paragraphs wherein the compound is of the formula (A2): 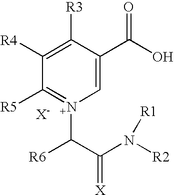
   wherein X (i.e. the ═X) is O, S, CH₂, or (H, H);
   wherein one of R1 and R2 is H, Me, Et, and the other of R1 and R2 is a C5-9 hydrocarbyl group or a C5-9 oxyhydrocarbyl group;
   wherein R3 is H or a hydrocarbyl group or a halo group;
   wherein R4 is H or a hydrocarbyl group or a halo group;
   wherein R5 is H or a hydrocarbyl group or a halo group;
   wherein R6 is H or a hydrocarbyl group or a halo group;
   wherein X⁻ is an anion, preferably a halo anion, preferably Br—;
   wherein the compound may be salt form or as a Zwitterion.
   Preferred Aspects

Certain preferred aspects of the present invention will now be described by way of numbered paragraphs.
1. A compound of formula (A1): 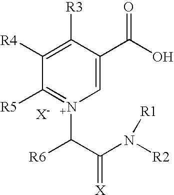
wherein X (i.e. the ═X) is O, S, CH2 or (H,H);
wherein R1 is hydrocarbyl group or an oxyhydrocarbyl group or H;
wherein R2 is hydrocarbyl group or an oxyhydrocarbyl group or H;
wherein R3 is H or halo or a hydrocarbyl group;
wherein R4 is H or halo or a hydrocarbyl group;
wherein R5 is H or halo or a hydrocarbyl group;
wherein R6 is H or halo or a hydrocarbyl group;
wherein X⁻ is an optional anion, such as Br⁻;
wherein the compound may be salt form or as a Zwitterion.
2. A compound according to paragraph 1 wherein the compound is of the formula (A2) 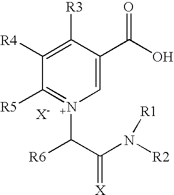
wherein X (i.e. the ═X) is O, S, CH₂, or (H, H);
wherein one of R1 and R2 is H or a hydrocarbyl group, and the other of R1 and R2 is a C5-C9 hydrocarbyl group or a C5-9 oxyhydrocarbyl group;
wherein R3 is H, halo or a hydrocarbyl group or a halo group;
wherein R4 is H, halo or a hydrocarbyl group or a halo group;
wherein R5 is H, halo or a hydrocarbyl group or a halo group;
wherein R6 is H, halo or a hydrocarbyl group or a halo group;
wherein X⁻ is an anion, preferably a halo anion, preferably Br—;
wherein the compound may be salt form or as a Zwitterion.
3. A compound according to any one of the preceding paragraphs wherein the compound is of the formula (A3) 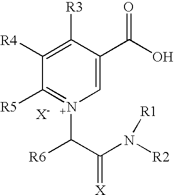
wherein X (i.e. the ═X) is O, S, CH₂, or (H, H);
wherein one of R1 and R2 is H or Me or Et, and the other of R1 and R2 is a C5-9 hydrocarbyl group or a C5-9 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.
4. A compound according to any one of the preceding paragraphs wherein the compound is of the formula (A4) 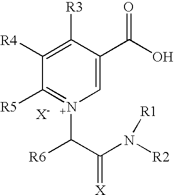
wherein X (i.e. the ═X) is O, S, CH₂, or (H, H);
wherein one of R1 and R2 is H or Me or Et, and the other of R1 and R2 is a C5-9 hydrocarbyl group or a C5-9 oxyhydrocarbyl group;
wherein R3 is H or a hydrocarbyl group;
wherein R4 is H or a hydrocarbyl group;
wherein R5 is H or a hydrocarbyl group;
wherein R6 is H or a hydrocarbyl group;
wherein X⁻ is an anion, preferably a halo anion, preferably Br—;
wherein the compound may be salt form or as a Zwitterion.
5. A compound according to any one of the preceding paragraphs wherein the compound is of the formula (A5) 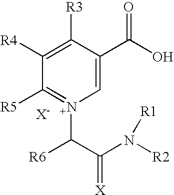
wherein X (i.e. the ═X) is O, S, CH₂, or (H, H);
wherein one of R1 and R2 is H or Me or Et, and the other of R1 and R2 is a C5-9 hydrocarbyl group or a C5-9 oxyhydrocarbyl group;
wherein R3 is H;
wherein R4 is H;
wherein R5 is H;
wherein R6 is H;
wherein X⁻ is an anion, preferably a halo anion, preferably Br—;
wherein the compound may be salt form or as a Zwitterion.
6. A compound according to any one of the preceding paragraphs wherein the compound is of the formula (A6) 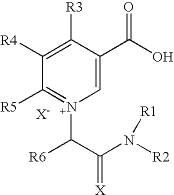
wherein X (i.e. the ═X) is O, S, CH₂, or (H, H);
wherein one of R1 and R2 is H, and the other of R1 and R2 is a C5-9 hydrocarbyl group or a C5-9 oxyhydrocarbyl group;
wherein R3 is H;
wherein R4 is H;
wherein R5 is H;
wherein R6 is H;
wherein X⁻ is an anion, preferably a halo anion, preferably Br—;
wherein the compound may be salt form or as a Zwitterion.
7. A compound according to any one of the preceding paragraphs wherein the compound is of the formula (A7) 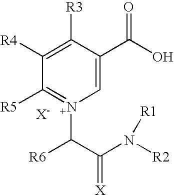
wherein X (i.e. the ═X) is O, S, CH₂, or (H, H);
wherein one of R1 and R2 is H or Me or Et, and the other of R1 and R2 is a C5 hydrocarbyl group or a C5 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.
8. A compound according to any one of the preceding paragraphs wherein the compound is of the formula (A8) 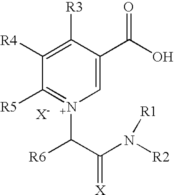
wherein X (i.e. the ═X) is O, S, CH₂, or (H, H);
wherein one of R1 and R2 is H or Me or Et, and the other of R1 and R2 is a C6 hydrocarbyl group or a C6 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.
9. A compound according to any one of the preceding paragraphs wherein the compound is of the formula (A9) 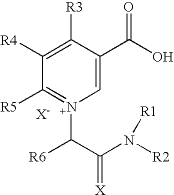
wherein X (i.e. the ═X) is O, S, CH₂, or (H, H);
wherein one of R1 and R2 is H or Me or Et, and the other of R1 and R2 is a C7 hydrocarbyl group or a C7 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.
10. A compound according to any one of the preceding paragraphs wherein the compound is of the formula (A10) 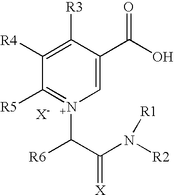
wherein X (i.e. the ═X) is O, S, CH₂, or (H, H);
wherein one of R1 and R2 is H or Me or Et, and the other of R1 and R2 is a C8 hydrocarbyl group or a C8 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.
11. A compound according to any one of the preceding paragraphs wherein the compound is of the formula (A1) 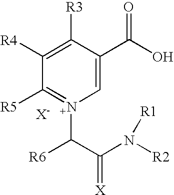
wherein X (i.e. the ═X) is O, S, CH₂, or (H, H);
wherein one of R1 and R2 is H or Me or Et, and the other of R1 and R2 is a C9 hydrocarbyl group or a C9 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion.
12. A compound according to any one of the preceding paragraphs wherein the compound is of the formula (B) (BZ52) 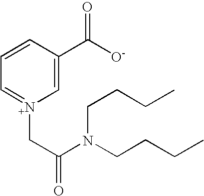
13. A compound according to any one of the preceding paragraphs wherein the compound is of the formula (D) (BZ194) 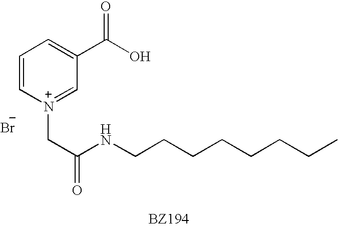
14. A compound according to any one of the preceding paragraphs wherein the compound is of the formula (E) (BZ320) 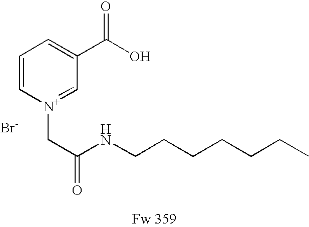
15. A compound according to any one of the preceding paragraphs wherein the compound is of the formula (F) (BZ321) 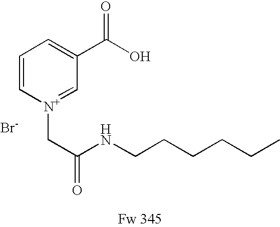
16. The use of a compound according to any one of the preceding paragraphs or a pharmaceutically acceptable salt thereof;
in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
   17. A pharmaceutical composition comprising a compound as defined in any of the preceding paragraphs or a pharmaceutically acceptable salt thereof admixed with a pharmaceutically acceptable carrier, diluent or excipient.
   18. A compound as defined in any of the preceding paragraphs or a pharmaceutically acceptable salt thereof for use in medicine.
   19. A medicament comprising a compound as defined in any of the preceding paragraphs or a pharmaceutically acceptable salt thereof.
   20. A method of treating and/or preventing a disease in a human or animal patient in need of same which method comprises administering to the patient an effective amount of a compound as defined in any of the preceding paragraphs or a pharmaceutically acceptable salt thereof.
   21. A process of preparing a pharmaceutical composition, said process comprising admixing one or more of the compounds defined in any of the preceding paragraphs or a pharmaceutically acceptable salt thereof with a pharmaceutically acceptable diluent, excipient or carrier.
   22. A pharmaceutical pack comprising one or more compartments, wherein at least one compartment comprises one or more of the compound as defined in any of the preceding paragraphs or a pharmaceutically acceptable salt thereof.
   23. A container comprising a compound as defined in any of the preceding paragraphs or a pharmaceutically acceptable salt thereof, wherein said container is labelled for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
   24. Use of a compound according to any one of the formulae presented herein in the manufacture of a medicament to affect the motility of T_{MBP-GFP} cells.
   25. A method of affecting the motility of T_{MBP-GFP} cells comprising administering a compound according to any one of the formulae presented herein.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, molecular biology or related fields are intended to be within the scope of the following claims.

1. The use of a compound of formula (I): 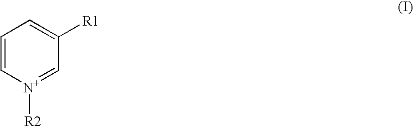
wherein:
R1 comprises a carbonyl group
R2 is a hydrocarbyl group;
optionally wherein said ring is further substituted;
or a pharmaceutically acceptable salt thereof;
in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

2. Use according to claim 1 wherein said compound is cell permeable.

3. Use according to claim 1 wherein said compound has a relative molecular mass (RMM) of less than about 500.

4. Use according to claim 1 wherein R2 is a hydrocarbyl group comprising a carbonyl group.

5. Use according to claim 1 wherein R2 is a hydrocarbyl group comprising an amide group.

6. Use according to claim 1 wherein R2 is CH₂C(O)NH₂.

7. Use according to claim 1 wherein R1 is COOH.

8. Use according to claim 1 wherein said compound is a mimetic of nicotinic group of NAADP, wherein said NAADP has the formula:

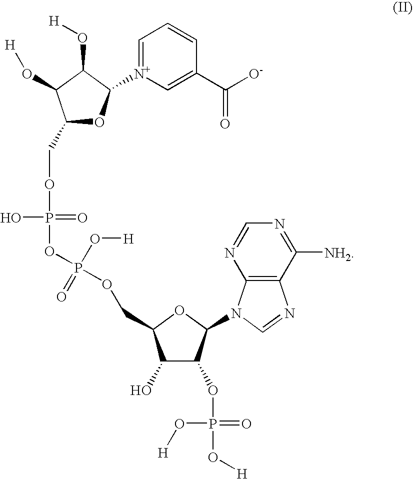

9. A pharmaceutical composition comprising a compound as defined in claim 1 or a pharmaceutically acceptable salt thereof admixed with a pharmaceutically acceptable carrier, diluent or excipient.

10. A pharmaceutical composition according to claim 9 wherein said composition comprises one or more additional pharmaceutically active compounds.

11. A compound of formula (I) or a pharmaceutically acceptable salt thereof as defined in claim 1 for use in medicine.

12. A compound of formula (I) or a pharmaceutically acceptable salt thereof as defined in claim 1.

13. A medicament comprising a compound as defined in claim 1.

14. An assay method for identifying an agent that modulates intracellular calcium release comprising the steps of:
(a) providing an agent;
(b) providing an NAADP receptor;
(c) contacting said agent with an NAADP receptor; and
(d) measuring the level of intracellular calcium release;
wherein a difference between (i) the level of intracellular calcium release in the presence of the agent; and (ii) the level of intracellular calcium release in the absence of the agent is indicative of an agent that modulates intracellular calcium release and may be useful in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

15. An assay method according to claim 14 wherein said agent is cell permeable.

16. An assay method according to claim 14 wherein said agent has a relative molecular mass (RMM) of less than about 500.

17. An assay method according to claim 14 wherein said agent is a mimetic of nicotinic group of NAADP, wherein said NAADP has the formula:

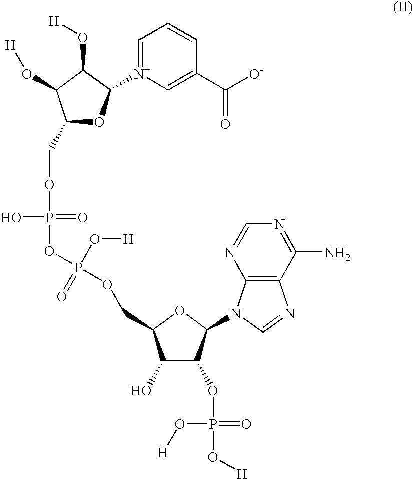

18. An assay method comprising the steps of:
(a) performing the assay method according to claim 14;
(b) identifying one or more agents capable of modulating intracellular calcium release; and
(c) preparing a quantity of those one or more identified agents.

19. A method comprising the steps of:
(a) performing the assay method according to claim 14;
(b) identifying one or more agents capable of modulating intracellular calcium release; and
(c) preparing a pharmaceutical composition comprising those one or more identified agents.

20. An agent identifiable, preferably, identified by the assay method according to claim 14.

21. A pharmaceutical composition prepared by the method of claim 19.

22. A method of treating and/or preventing a disease in a human or animal patient in need of same which method comprises administering to the patient an effective amount of a compound as defined in claim 1.

23. A method of treating and/or preventing a disease in a human or animal patient in need of same which method comprises administering to the patient an effective amount of a composition according to claim 9.

24. A method of treating and/or preventing a disease in a human or animal patient in need of same which method comprises administering to the patient an effective amount of a medicament according to claim 13.

25. (canceled)

26. A process of preparing a pharmaceutical composition, said process comprising admixing one or more of the compounds defined in claim 1 with a pharmaceutically acceptable diluent, excipient or carrier.

27. A pharmaceutical pack comprising one or more compartments, wherein at least one compartment comprises one or more of the compounds defined in claim 1.

28. A pharmaceutical pack comprising one or more compartments, wherein at least one compartment comprises a composition according to claim 9.

29. A pharmaceutical pack comprising one or more compartments, wherein at least one compartment comprises a medicament according to claim 13.

30. A container comprising a compound according to claim 1, wherein said container is labelled for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

31. A container comprising a medicament according to claim 13, wherein said container is labelled for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

32. The use of a compound of formula (I) as defined in claim 1 in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
wherein said compound is cell permeable;
wherein said compound has a relative molecular mass of less than about 500;
wherein said compound is a mimetic of the nicotinic group of NAADP, wherein said NAADP has the formula:

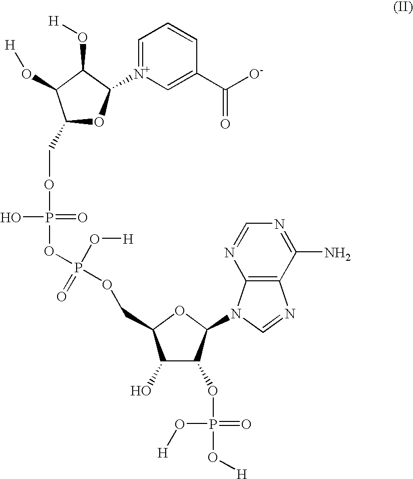

33. The use of a compound in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
wherein said compound is cell permeable;
wherein said compound has a relative molecular mass of less than about 500;
wherein said compound is a mimetic of the nicotinic group of NAADP, wherein said NAADP has the formula: 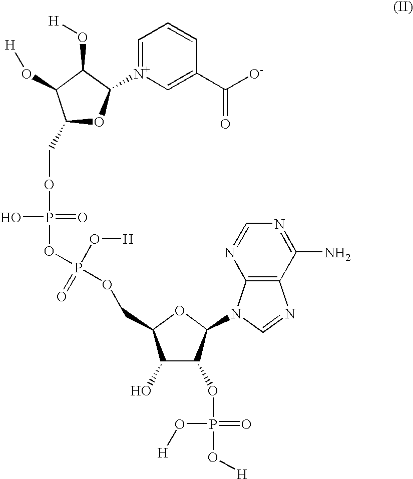
and wherein said compound has the formula (A): 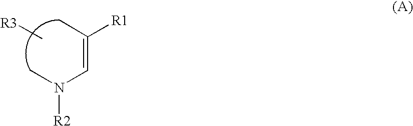
wherein R1 and R2 are ring substituents;
wherein R1 and R2 are as defined for compound of formula (I); and
wherein R3 represents one or more substituents, the or each substituent being independently selected from H, substituted or unsubstituted aryl, C1-20 alkyl, F, Cl, Br, I, OY, NYₙ (n=1, 2, or 3), SY, COY, CONYₙ (z=2), C(O)OY, wherein the or each Y is independently selected from H, a substituted or unsubstituted aryl, and a C1-20 alkyl group.

34. A compound for use in medicine
wherein said compound is cell permeable;
wherein said compound has a relative molecular mass of less than about 500;
wherein said compound is a mimetic of the nicotinic group of NAADP, wherein said NAADP has the formula: 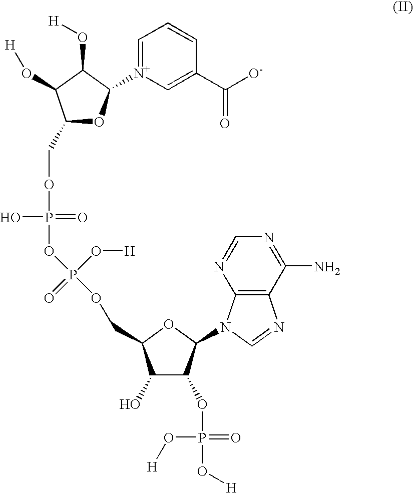
and wherein said compound has the formula (A): 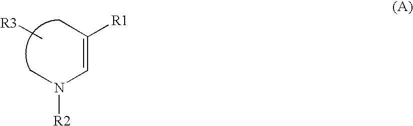
wherein R1 and R2 are ring substituents;
wherein R1 and R2 are as defined for compound of formula (I); and
wherein R3 represents one or more substituents, the or each substituent being independently selected from H, substituted or unsubstituted aryl, C1-20 alkyl, F, Cl, Br, I, OY, NYₙ (n=1, 2, or 3), SY, COY, CONY_{z} (z=2), C(O)OY, wherein the or each Y is independently selected from H, a substituted or unsubstituted aryl, and a C1-20 alkyl group.

35. A compound, or a pharmaceutical composition comprising same,
wherein said compound is cell permeable;
wherein said compound has a relative molecular mass of less than about 500;
wherein said compound is a mimetic of the nicotinic group of NAADP, wherein said NAADP has the formula: 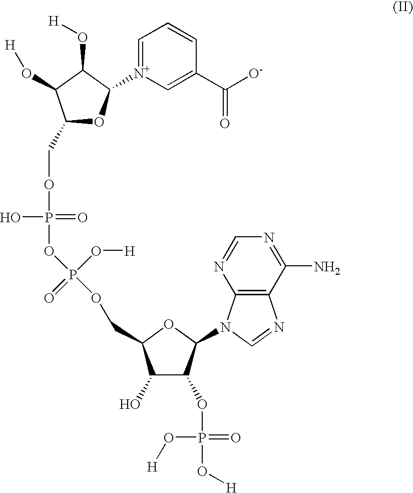
and wherein said compound has the formula (A): 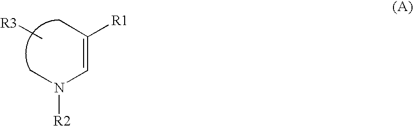
wherein R1 and R2 are ring substituents;
wherein R1 and R2 are as defined for compound of formula (1); and
wherein R3 represents one or more substituents, the or each substituent being independently selected from H, substituted or unsubstituted aryl, C1-20 alkyl, F, Cl, Br, I, OY, NYₙ (n=1, 2, or 3), SY, COY, CONYₙ (z=2), C(O)OY, wherein the or each Y is independently selected from H, a substituted or unsubstituted aryl, and a C1-20 alkyl group.

36. The use according to claim 1 wherein said compound has the formula:

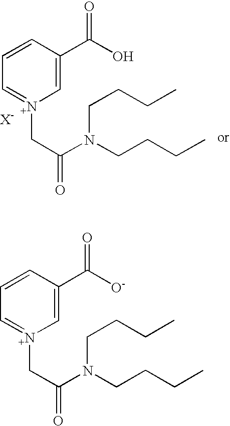

37. The pharmaceutical composition according to claim 9 wherein said compound has the formula:

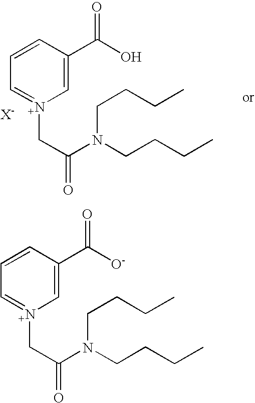

38. The method according to claim 22 wherein said compound has the formula:

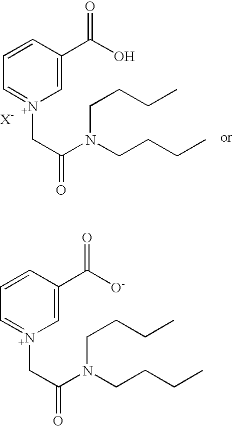

39. The method according to claim 26 wherein said compound has the formula:

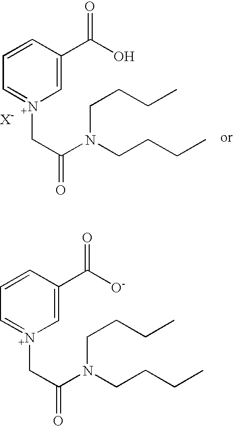

40. The container according to claim 30 wherein said compound has the formula:

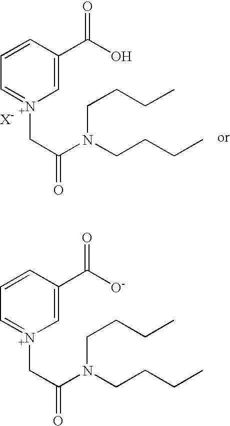

41. The use of a compound of formula (A1): 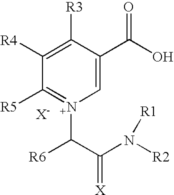
wherein X (of the ═X group) is O, S, CH2 or (H,H);
wherein R1 is hydrocarbyl group or an oxyhydrocarbyl group or H;
wherein R2 is hydrocarbyl group or an oxyhydrocarbyl group or H;
optionally wherein R1 and R2 can be linked so as to form a hydrocarbyl ring structure
wherein R3 is H or halo or a hydrocarbyl group;
wherein R4 is H or halo or a hydrocarbyl group;
wherein R5 is H or halo or a hydrocarbyl group;
wherein R6 is H or halo or a hydrocarbyl group;
wherein X⁻ is an optional anion;
wherein the compound may be in the form of a pharmaceutically acceptable salt thereof salt form or as a Zwitterion;
in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

42. Use according to claim 41 wherein said compound is cell permeable.

43. Use according to claim 41 wherein said compound has a relative molecular mass (RMM) of less than about 500.

44. A pharmaceutical composition comprising a compound as defined in claim 41 or a pharmaceutically acceptable salt thereof admixed with a pharmaceutically acceptable carrier, diluent or excipient.

45. A pharmaceutical composition according to claim 44 wherein said composition comprises one or more additional pharmaceutically active compounds.

46. A compound of formula (A1) or a pharmaceutically acceptable salt thereof as defined in claim 41 for use in medicine.

47. A compound of formula (A1) or a pharmaceutically acceptable salt thereof as defined in claim 41.

48. A medicament comprising a compound as defined in claim 41.

49. A method of treating and/or preventing a disease in a human or animal patient in need of same which method comprises administering to the patient an effective amount of a compound as defined in claim 41.

50. A method of treating and/or preventing a disease in a human or animal patient in need of same which method comprises administering to the patient an effective amount of a composition according to claim 44.

51. A method of treating and/or preventing a disease in a human or animal patient in need of same which method comprises administering to the patient an effective amount of a medicament according to claim 47.

52. A process of preparing a pharmaceutical composition, said process comprising admixing one or more of the compounds defined in claim 41 with a pharmaceutically acceptable diluent, excipient or carrier.

53. A pharmaceutical pack comprising one or more compartments, wherein at least one compartment comprises one or more of the compounds defined in claim 41.

54. A pharmaceutical pack comprising one or more compartments, wherein at least one compartment comprises a composition according to claim 44.

55. A pharmaceutical pack comprising one or more compartments, wherein at least one compartment comprises a medicament according to claim 47.

56. A container comprising a compound according to claim 41, wherein said container is labelled for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

57. A container comprising a medicament according to claim 47, wherein said container is labelled for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

58. The use of a compound of formula (A1) as defined in claim 41 in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.
wherein said compound is cell permeable;
wherein said compound has a relative molecular mass of less than about 500;
wherein said compound is a mimetic of the nicotinic group of NAADP, wherein said NAADP has the formula:

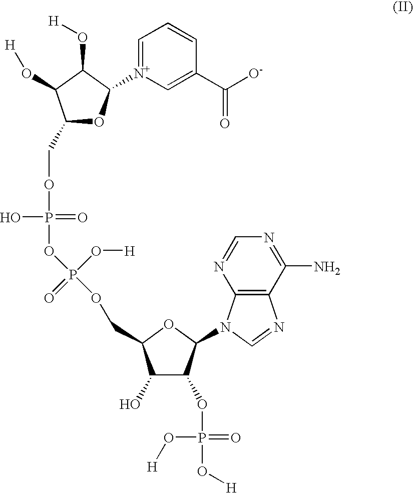

59. The use of a compound of formula (A1): 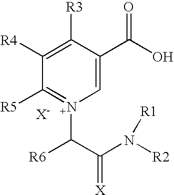
wherein X (of the ═X group) is O, S, CH2 or (H,H);
wherein R1 is hydrocarbyl group or an oxyhydrocarbyl group or H;
wherein R2 is hydrocarbyl group or an oxyhydrocarbyl group or H;
optionally wherein R1 and R2 can be linked so as to form a hydrocarbyl ring structure
wherein R3 is H or halo or a hydrocarbyl group;
wherein R4 is H or halo or a hydrocarbyl group;
wherein R5 is H or halo or a hydrocarbyl group;
wherein R6 is H or halo or a hydrocarbyl group;
wherein X⁻ is an optional anion;
wherein the compound may be in the form of a pharmaceutically acceptable salt thereof salt form or as a Zwitterion;
in the manufacture of a medicament for use in treating RA (rheumatoid arthritis) or MS (multiple sclerosis) or graft rejection.

60. Use according to claim 59 wherein said compound is cell permeable.

61. Use according to claim 59 wherein said compound has a relative molecular mass (RMM) of less than about 500.

62. A method of treating and/or preventing RA (rheumatoid arthritis) or MS (multiple sclerosis) or graft rejection in a human or animal patient in need of same which method comprises administering to the patient an effective amount of a compound as defined in claim 59.

63. A container comprising a compound according to claim 59, wherein said container is labelled for use in treating RA (rheumatoid arthritis) or MS (multiple sclerosis) or graft rejection.

64. The use of a compound of formula (A1) as defined in claim 59 in the manufacture of a medicament for use in treating RA (rheumatoid arthritis) or MS (multiple sclerosis) or graft rejection
wherein said compound is cell permeable;
wherein said compound has a relative molecular mass of less than about 500;
wherein said compound is a mimetic of the nicotinic group of NAADP, wherein said NAADP has the formula:

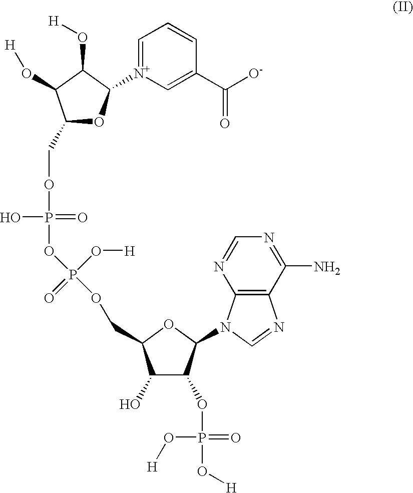

65. The use according to claim 41 wherein said compound has the formula:

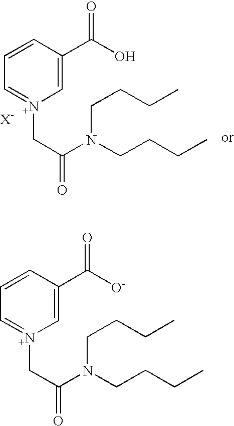

66. The pharmaceutical composition according to claim 44 wherein said compound has the formula:

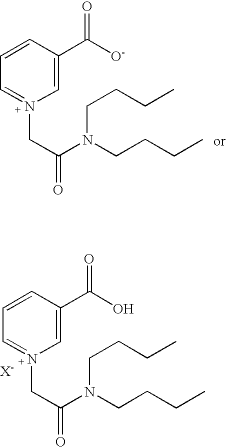

67. The method according to claim 49 wherein said compound has the formula:

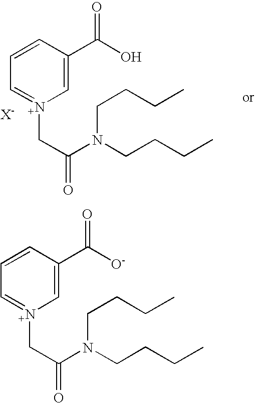

68. The use according to claim 59 wherein said compound has the formula:

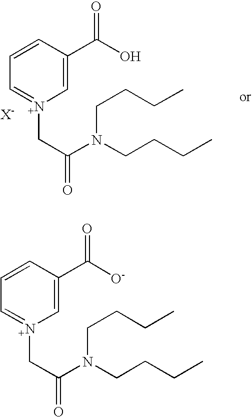

69. The use according to claim 64 wherein said compound has the formula:

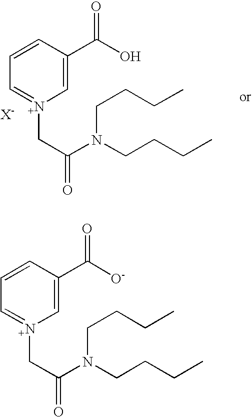

70. The method according to claim 62 wherein said compound has the formula:

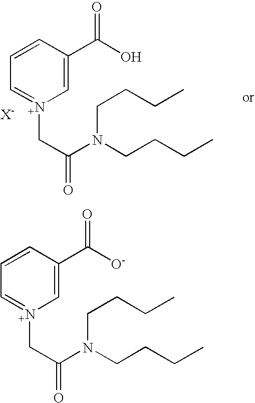

71. (canceled)

72. The use according to claim 1 wherein said compound has the formula:

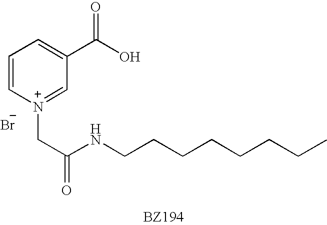

73. The pharmaceutical composition according to claim 9 wherein said compound has the formula:

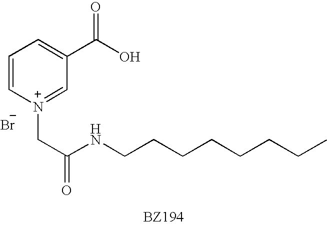

74. The method according to claim 22 wherein said compound has the formula:

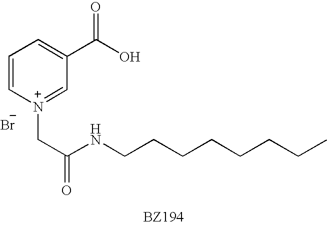

75. The method according to claim 26 wherein said compound has the formula:

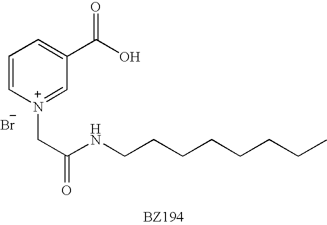

76. The container according to claim 30 wherein said compound has the formula:

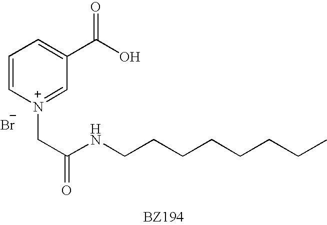

77. The use of a compound of formula (A2): 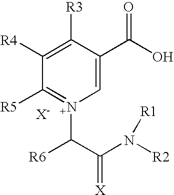
wherein X (i.e. the ═X) is O, S, CH₂, or (H, H);
wherein one of R1 and R2 is H, Me, Et, and the other of R1 and R2 is a C5-9 hydrocarbyl group or a C5-9 oxyhydrocarbyl group;
wherein R3 is H or a hydrocarbyl group or a halo group;
wherein R4 is H or a hydrocarbyl group or a halo group;
wherein R5 is H or a hydrocarbyl group or a halo group;
wherein R6 is H or a hydrocarbyl group or a halo group;
wherein X⁻ is an anion, preferably a halo anion, preferably Br—;
wherein the compound may be salt form or as a Zwitterion;
in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

78. Use according to claim 77 wherein said compound is cell permeable.

79. Use according to claim 77 wherein said compound has a relative molecular mass (RMM) of less than about 500.

80. A pharmaceutical composition comprising a compound as defined in claim 77 or a pharmaceutically acceptable salt thereof admixed with a pharmaceutically acceptable carrier, diluent or excipient.

81. A pharmaceutical composition according to claim 77 wherein said composition comprises one or more additional pharmaceutically active compounds.

82. A compound of formula (A2) or a pharmaceutically acceptable salt thereof as defined in claim 77 for use in medicine.

83. A compound of formula (A2) or a pharmaceutically acceptable salt thereof as defined in claim 77.

84. A medicament comprising a compound as defined in claim 77.

85. A method of treating and/or preventing a disease in a human or animal patient in need of same which method comprises administering to the patient an effective amount of a compound as defined in claim 77.

86. A method of treating and/or preventing a disease in a human or animal patient in need of same which method comprises administering to the patient an effective amount of a composition according to claim 77.

87. A method of treating and/or preventing a disease in a human or animal patient in need of same which method comprises administering to the patient an effective amount of a medicament according to claim 77.

88. A process of preparing a pharmaceutical composition, said process comprising admixing one or more of the compounds defined in claim 77 with a pharmaceutically acceptable diluent, excipient or carrier.

89. A pharmaceutical pack comprising one or more compartments, wherein at least one compartment comprises one or more of the compounds defined in claim 77.

90. A pharmaceutical pack comprising one or more compartments, wherein at least one compartment comprises a composition according to claim 77.

91. A pharmaceutical pack comprising one or more compartments, wherein at least one compartment comprises a medicament according to claim 77.

92. A container comprising a compound according to claim 77, wherein said container is labelled for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

93. A container comprising a compound as defined in claim 77, wherein said container is labelled for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells.

94. The use of a compound of formula (A2) as defined in claim 77 in the manufacture of a medicament for use in one or more of:
modulating the release of intracellular calcium from a store controlled by nicotinic acid adenine dinucleotide phosphate
modulating calcium spikes and sustained elevations in the free cytosolic and nuclear calcium concentration in mammalian cells
treating diseases in one or more of brain, heart, pancreatic cells (e.g. pancreatic acinar and pancreatic beta cells), immune cells, such as T-cells, and other haemopoietic cells including phagocytes
treating diseases in one or more of brain, heart, pancreatic cells (e.g. pancreatic acinar and pancreatic beta cells), immune cells, such as T-cells, and other haemopoietic cells including phagocytes by modulating the release of intracellular calcium from a store controlled by nicotinic acid adenine dinucleotide phosphate
treating diseases in one or more of brain, heart, pancreatic cells (e.g. pancreatic acinar and pancreatic beta cells), such as T-cells, and other haemopoietic cells including phagocytes by modulating calcium spikes in mammalian cells.
treating diseases in one or more of brain, heart, pancreatic cells (e.g. pancreatic acinar and pancreatic beta cells), such as T-cells, and other haemopoietic cells including phagocytes by modulating sustained elevations in the free cytosolic and nuclear calcium concentration in mammalian cells
wherein said compound is cell permeable;
wherein said compound has a relative molecular mass of less than about 500;
wherein said compound is a mimetic of the nicotinic group of NAADP, wherein said NAADP has the formula:

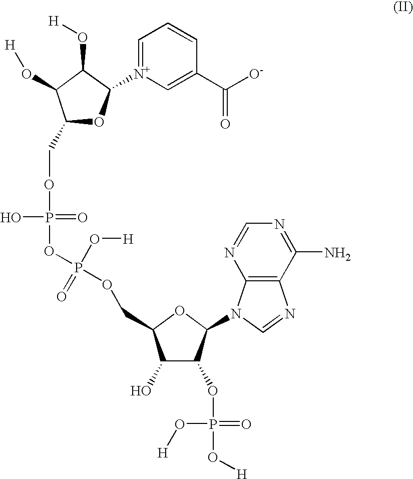

95. The use of a compound of formula (A2) as defined in claim 77 wherein the compound may be in the form of a pharmaceutically acceptable salt thereof salt form or as a Zwitterion;
in the manufacture of a medicament for use in treating RA (rheumatoid arthritis) or MS (multiple sclerosis) or graft rejection.

96. Use according to claim 95 wherein said compound is cell permeable.

97. Use according to claim 95 wherein said compound has a relative molecular mass (RMM) of less than about 500.

98. A method of treating and/or preventing RA (rheumatoid arthritis) or MS (multiple sclerosis) or graft rejection in a human or animal patient in need of same which method comprises administering to the patient an effective amount of a compound as defined in claim 77.

99. A container comprising a compound according to claim 77, wherein said container is labelled for use in treating RA (rheumatoid arthritis) or MS (multiple sclerosis) or graft rejection.

100. The use of a compound of formula (A2) as defined in claim 77 in the manufacture of a medicament for use in treating RA (rheumatoid arthritis) or MS (multiple sclerosis) or graft rejection
wherein said compound is cell permeable;
wherein said compound has a relative molecular mass of less than about 500;
wherein said compound is a mimetic of the nicotinic group of NAADP, wherein said NAADP has the formula:

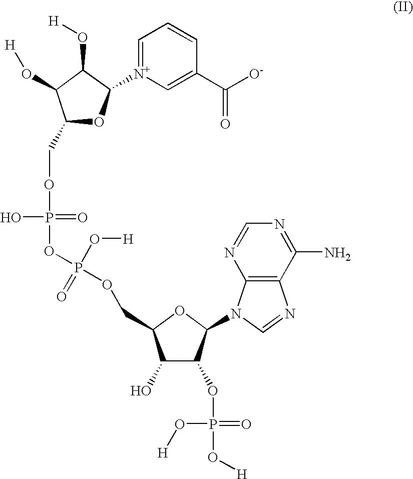

101. Use of a compound according to Formula I or Formula A or any one of Formula A1-Formula A11 or any one of Formula B-Formula F in the manufacture of a medicament to affect the motility of T_{MBP-GFP} cells.

102. A method of affecting the motility of T_{MBP-GFP} cells comprising administering a compound according to Formula I or Formula A or any one of Formula A1-Formula A 11 or any one of Formula B-Formula F.
